(19) United States
(12) Patent Application Publication
(10) Pub. No.: US 2006/0222647 A1
(43) Pub. Date: Oct. 5, 2006

(54) Methods and compositions for modulating the activity of PDE5

(76) Inventors: Joseph A. Beavo, seattle, WA (US); Jackie D. Corbin, Nashville, TN (US); Kenneth M. Ferguson, Bothell, WA (US); Sharron H. Francis, Nashville, TN (US); Ann Kadlecek, Madison, CT (US); Linda M. McAllister-Lucas, Ann Arbor, MI (US); Kate Loughney, Seattle, WA (US); William K. Sonnenburg, Spring, TX (US); Melissa K. Thomas, Boston, MA (US); Sergei D. Rybalkin, Seattle, WA (US)

Correspondence Address: MILES & STOCKBRIDGE PC, 1751 PINNACLE DRIVE, SUITE 500, MCLEAN, VA, 22102-3833, US

(21) Appl. No.: 10/352,534

(22) Filed: Jan. 28, 2003

Related U.S. Application Data
(63) Continuation-in-part of application No. 10/115,515, filed on Apr. 3, 2002, now abandoned.
Application No. 10/115,515 is a continuation of application No. 09/599,658, filed on Jun. 21, 2000, now abandoned.
Application No. 09/599,658 is a continuation of application No. 09/055,584, filed on Apr. 6, 1998, now abandoned.
Application No. 09/055,584 is a division of application No. 08/463,949, filed on Jun. 5, 1995, now Pat. No. 5,955,583.
Application No. 08/463,949 is a continuation-in-part of application No. 08/068,051, filed on May 27, 1993, now abandoned.

(51) Int. Cl. A61K 48/00; A61K 38/54; A61K 39/395

(52) U.S. Cl. 424/146.1; 514/2; 514/44

(57) ABSTRACT

The present invention provides agents such as agonists, antibodies, antagonists or inhibitors to modulate the activity of PDE5 proteins. These compositions and methods are useful for the diagnosis or treatment of conditions associated with the presence, the deficiency, altered levels, or altered activity of PDE5 proteins.

This application is a continuation-in-part of of copending U.S. Ser. No. 10/115,515 filed Apr. 3, 2002, which is a continuation of U.S. Ser. No. 09/599,658 filed Jun. 21, 2000, which is a continuation of U.S. Ser. No. 09/055,584 filed Apr. 6, 1998, which is a divisional of U.S. Ser. No. 08/463,949 filed Jun. 5, 1995, which is a CIP of U.S. Ser. No. 08/068,051 filed May 27, 1993, now abandoned, the contents of which are all hereby incorporated by reference, in their entirety, into this application.

This work was supported by Research Grant DK 21723 and HL 44948 from the National Institutes of Health.

Throughout this application various publications are referenced. The disclosures of these publications in their entireties are hereby incorporated by reference into this application in order to more fully describe the state of the art to which this invention pertains.

### FIELD OF THE INVENTION

The present invention provides methods and compositions for modulating the enzymatic activity of PDE5 and more particularly to the development of specific agents that modulate the enzymatic activity of PDE5, thereby targeting PDE5 mediated diseases.

### BACKGROUND OF THE INVENTION

The secondary messengers cAMP and cGMP, play important roles in mediating the biological effects of a wide variety of first messengers such as transducing a variety of extracellular signals, including hormones, light, and neurotransmitters. The intracellular levels of cAMP and cGMP are controlled by their rates of synthesis by cyclases and their rate of degradation by phosphodiestrases (PDEs).

Multiple families of PDEs have been identified (Beavo, J. A. (1995) Physiol. Rev. 75, 725-748; Soderling, S. H., Bayuga, S. J., Beavo, J. A. (1998) J. Biol. Chem. 273, 15553-15558; Fisher, D. A., Smith, J. F., Pillar, J. S., St. Denis, S. H., Cheng, J. B. (1998) J. Biol. Chem. 273, 15559-15564). Most of these families contain distinct genes, many of which are expressed in different tissues as alternative splice variants. Each PDE family has multiple isozymes and multiple splice variants displaying characteristic kinetic and regulatory properties, sequence homologies, and inhibitor profiles. Several lines of evidence have established an important role for PDEs in a wide variety of physiological processes. Genetic studies have indicated that different PDEs regulate such processes as learning and memory (Kauvar, L. M. (1982) J. Neurosci. 2, 1347-1358), development (Shaulsky, G., Escalante, R., Loomis, W. F. (1996) Proc. Natl. Acad. Sci. USA 93, 15260-15265), and visual signal transduction (McLaughlin, M. E., Sandberg, M. A., Berson, E. L., Dryja, T. P. (1993) Nat. Genet. 4, 130-134). Molecular and pharmacological studies have suggested that PDEs regulate such disparate functions as platelet aggregation (Dickinson, N. T., lang, E. K., Hasalam, R. J. (1997) Biochem. J. 323, 371-377), aldosterone production, (MacFarland, R. T., Zelus, B. D., Beavo, J. A. (1991) J. Biol. Chem, 266, 136-142), insulin secretion (Zhao, A. Z., Zhao, H., Teague, J., Fujimoto, W., Beavo, J. A. (1997) Proc. Natl. Acad. Sci. USA 942, 3223-3228), and olfactory signal transduction (Yan, C., Zhao, A. Z., Bentley, J. K., Loughney, K., Ferguson, K., Beavo, J. A. (1995) Proc. Natl. Acad. Sci. USA 92, 9677-9681).

PDEs are typically composed of a catalytic domain (approximately 270 amino acids), one or more N-terminal regulatory domains responsible for binding cofactors, and, in some cases, a C-terminal domain of unknown function. Based on their biological properties, the PDEs may be classified into a number of general families: the Ca²⁺/calmodulin-stimulated PDEs (Type 1), the cGMP-stimulated PDEs (Type 2), the cGMP-inhibited PDEs (Type 3), the cAMP-specific PDEs (Type 4), the cGMP-specific PDE cGB-PDE (Type 5) and the cGMP-specific photoreceptor PDEs (Type 6).

The cGMP-binding PDEs (Type 2, Type 5 and Type 6 PDEs) in addition to having a homologous catalytic domain near their carboxyl terminus, have a second conserved sequence which is located closer to their amino terminus and which may comprise one or two allosteric cGMP-binding domains. See Charbonneau et al., Proc. Natl. Acad. Sci. USA, 87: 288-292 (1990).

A variety of diseases have been suggested to result from decreased levels of cyclic nucleotides as a result of increased PDE activity. For example, altered PDE3 has been associated with cardiac disease (Smith, C. J., Huang, R., Sun, D., Ricketts, S., Hoegler, C., Ding, J. Z., Moggio, R. A., Hintze, T. H. (1997) Circulation 96, 3116-23). A form of diabetes insipidus in the mouse has been associated with increased PDE4 activity (Dousa, T. P. (1999) Kidney Int. 55, 29-62). Furthermore, defects in PDE6 have also been associated with retinal diseases, such as retinal degeneration in mouse (Tsung, S. H., Gouras, P., Yamashita, C. K., Kjeldbye, H., Fisher, J., Farber, D. B., Goff, S. P. (1996) Science 272, 1026-9), autosomal recessive retinitis in humans (Baiget, M., Calaf, M., Valverde, D., del Rio, E., Reig, C., Carballo, M., Calvo, M. T., Gonzales-Duarte, R. (1998) Med. Clin. 111, 420-422), and rod/cone dysplasia in some dogs (Dekomien, G., Epplen, J. T. (2000) Anim. Genet. 31, 135-139).

cGMP-specific, cGMP-binding phosphodiesterase (cGB-PDE; also known as PDE5), the subject of the present invention, has been implicated in regulation of such physiological processes as smooth muscle relaxation and neuronal survival. For example, a variety of physiological processes in the cardiovascular, nervous and immune systems are controlled by the nitric oxide/cGMP signaling pathway. In smooth muscle NO and natriuretic peptides regulate vascular tone by inducing relaxation through stimulation of cGMP synthesis (Sausbier et al., (2000) Circ Res, 87, 825-830). Degradation of cGMP is controlled by cyclic nucleotide phosphodiesterases, and PDE5 (cGMP-specific, cGMP-binding PDE5) is the most highly expressed cGMP hydrolyzing PDE in these cells.

The mechanisms by which PDE5 activity is regulated in the cell have not been completely resolved. PDE5 has at least one and possibly two cGMP-binding sites on its the N-terminal end (McAllister-Lucas et al., (1993) J Biol Chem, 268, 22863-22873). Recently, these sites have been defined as two GAF domains (GAF A and GAF B) based on their sequence homology with similar motifs in a wide group of proteins. This group originally included cGMP regulated phophodiesterases (PDE2, PDE5, PDE5, PDE10 and PDE11), some adenylyl cyclases and Fh1A protein (a bacterial transcriptional factor), but now contains over a thousand proteins (Anantharaman et al., (2001) J Mol Biol, 307, 1271-1292; Aravind and Ponting, (1997) Trends Biochem Sci, 22, 458-459). However, the mere presence of this motif does not necessarily mean that all of these proteins are able to bind cGMP. For example, the GAF domain of cyanobacterial adenylyl cyclase (cyaBi) specifically binds cAMP (Kanacher et al., (2002) Embo J, 21, 3672-3680). The result of occupation of the GAF domain by cAMP, generated as a product of the adenylyl cyclase catalytic reaction, had a profound effect on the adenylyl cyclase catalytic activity, leading to a rapid exponential activation of this enzyme.

Among cGMP binding proteins, PDE2, PDE5 and PDE6 have been reported to have high affinity binding sites for cGMP. cGMP binding to the GAF domain of PDE2 greatly stimulates its cAMP hydrolytic (catalytic) activity (Martins et al., (1982) J Biol Chem, 257, 1973-1979; Stroop and Beavo, (1992) Adv Second Messenger Phosphoprotein Res, 25, 55-71). Because of the critical role of PDEs in intracellular signaling, efforts have focused on finding agents that selectively activate or inhibit specific PDE isozymes. Agents which affect cellular PDE activity, and thus alter cellular cAMP, can potentially be used to control a broad range of diseases and physiological conditions. Some drugs which raise cAMP levels by inhibiting PDEs are in use, but generally act as broad nonspecific inhibitors and have deleterious side effects on cAMP activity in nontargeted tissues and cell types. Accordingly, agents are still needed which are specific for selected PDE isozymes.

Selective inhibitors of specific PDE isozymes may be useful as cardiotonic agents, anti-depressants, anti-hypertensives, anti-thrombotics, and as other agents. Screening studies for agonists/antagonists have been complicated, however, because of difficulties in identifying the particular PDE isozyme present in a particular assay preparation. Moreover, all PDEs catalyze the same basic reaction; all have overlapping substrate specificities; and all occur only in trace amounts.

Several approaches have been attempted to differentiate among PDEs. The classical enzymological approach of isolating and studying each new isozyme is hampered by current limits of purification techniques and by the inability to accurately assess whether complete resolution of an isozyme has been achieved. A second approach has been to identify isozyme-specific assay conditions which might favor the contribution of one isozyme and minimize that of others. Another approach has been the immunological identification and separation into family groups and/or individual isozymes.

There are obvious problems with each of these approaches; for the unambiguous identification and study of a particular isozyme, a large number of distinguishing criteria need to be established, which is often time consuming and in some cases technically quite difficult. As a result, most studies have been done with only partially pure PDE preparations that probably contained more than one isozyme. Moreover, many of the PDEs in most tissues are very susceptible to limited proteolysis and easily form active proteolytic products that may have different kinetic, regulatory, and physiological properties from their parent form.

The structure-function analysis of PDE5 coupled with the ability to isolate large quantities of kinetically pure preparations of tissue-specific PDE5 by recombinant means, the subject of the present invention, should facilitate the development of new and specific PDE5-modulatory agents. More importantly, the ability to produce kinetically pure, PDE5 having a “non-activated native” conformation permits screening for PDE5-activating agents.

### SUMMARY OF THE INVENTION

Accordingly, this invention provides agents that modulate the activity of cGMP-binding, cGMP-specific phosphodiesterases (cGB-PDEs); also known as PDE5s, of the invention, and methods for their use to modulate the activity of PDE5.

The present invention also provides agents with which PDE5 protein will interact so as to modify its enzymatic activity. Agents that modulate PDE5 activity may be identified by incubating a putative modulator with lysate from eucaryotic cells expressing recombinant PDE5, and determining the effect of the putative modulator on PDE5 phosphodiesterase activity, using an assay for that activity. Agents that modulate PDE5 activity include agents that enhance or agonize PDE5 activity. In addition, agents that modulate PDE5 activity include agents that inhibit or antagonize PDE5 activity.

In an embodiment, an eukaryotic cell that expresses PDE5 but lacks appreciable endogenous cyclic nucleotide phosphodiesterase activity, is used to determine if a test compound modulates the activity of PDE5. Specifically illustrating such a eukaryotic cell is the yeast strain YKS45 which was deposited with the ATCC on May 19, 1993 as Accession No. 74225. The selectivity of a compound that modulates the activity of a PDE5 protein can be evaluated by comparing its activity on PDE5 to its activity on other PDE isozymes (for example, PDE5 vs PDE6 vs PDE9), splice variants (for example PDE5Alvs PDE5A2 vs PDE5A3), or a mutant PDE5. More importantly, the invention provides compositions, including various types of RNA based inhibitors expressed in whole cells to differentiate between isozymes such as PDE5 vs PDE6 vs PDE9, all of which are cGMP specific.

Once a compound that modulates the activity of a PDE5 is discovered, its selectivity can be evaluated by comparing its activity on the PDE5 to its activity on other PDE isozymes. Thus, the combination of the recombinant PDE5 products of the invention with other recombinant PDE products provides a system for developing selective modulators of PDE5.

Selective modulators of PDE5 may include, but are not limited to, for example, antibodies and other proteins or peptides which specifically bind to PDE5 protein or PDE5 nucleic acid, oligonucleotides which specifically bind to PDE5 protein or PDE5 nucleic acid and other non-peptide compounds (e.g., isloated or synthetic organic molecules) which specifically interact with PDE5 protein or PDE5 nucleic acid. Mutant forms of PDE5 which affect the enzymatic activity or cellular localization of the wild-type PDE5 are also contemplated by the invention.

The preferred targets for the development of selective modulators of the invention include, for example: (1) the regions of PDE5, which contact other proteins and/or localize PDE5 within a cell, (2) the regions of PDE5 which bind substrate (e.g., the catalytic site), (3) the allosteric cGMP-binding site(s) of PDE5 (e.g., GAF A or GAF B domain), (4) the phosphorylation site(s) of PDE5 and (5) the regions of PDE5 which are involved in dimerization/multimerization of PDE5 subunits.

In specific embodiments, the invention provides cGMP-binding sites within the GAF (GAF A and GAF B) domains of PDE5 as targets for development of both agonists and antagonists of PDE5 activity.

Agents that modulate PDE5 activity may be therapeutically useful in treatment of a wide range of diseases and physiological processes in a mammal, for example disease or dysfunction of the cardiovascular, nervous and immune systems that are controlled by nitric oxide/cGMP signaling pathways.

### BRIEF DESCRIPTION OF THE FIGURES

FIGS. 1A, 1B, and 1C is an alignment of the conserved catalytic domains of several PDE isoenzymes (residues which are identical in all PDEs listed are indicated by their one letter amino acid abbreviation in the “conserved” line, residues which are identical in the cGB-PDE and photoreceptor PDEs only are indicated by a star in the “conserved” line and gaps introduced for optimum alignment are indicated by periods.)

FIGS. 2A 2B, and 2C is an alignment of the cGMP-binding domains of several PDE isoenzymes (residues which are identical in all PDEs listed are indicated by their one letter amino acid abbreviation in the “conserved” line and gaps introduced for optimum alignment are indicated by periods.)

FIG. 3 is an alignment of internally homologous repeats from several PDE isoenzymes (residues identical in each repeat A and B from all cGMP-binding PDEs listed are indicated by their one letter amino acid abbreviation in the “conserved” line and stars in the “conserved” line represent positions in which all residues are chemically conserved.)

FIG. 4 schematically depicts the domain organization of cGB-PDE.

FIG. 5 is a bar graph representing the results of experiments in which extracts of COS cells transfected with bovine cGB-PDE sequences or extracts of untransfected COS cells were assayed for phosphodiesterase activity using either 20 μM cGMP or 20 μM cAMP as the substrate, as described in Example 5, infra.

FIG. 6 is a graph depicting results of assays of extracts from cells transfected with bovine cGB-PDE sequences for cGMP phosphodiesterase activity in the presence of a series of concentrations of phosphodiesterase inhibitors including dypyridamole (closed squares), zaprinast (closed circles), methoxymethylxanthine (closed triangles) and rolipram (open circles), as described in Example 5, infra.

FIG. 7 is a bar graph presenting results of experiments in which cell extracts from COS cells transfected with bovine cGB-PDE sequences or control untransfected COS cells were assayed for [³ H]cGMP-binding activity in the absence (−) or presence (+) of 0.2 mM IBMX, as described in Example 5, infra.

FIG. 8 is a graph of the results of assays in which extracts from cells transfected with bovine cGB-PDE sequences were assayed for [³H]cGMP-binding activity in the presence of excess unlabelled cAMP (open circles) or cGMP (closed circles) at the concentrations indicated, as described in Example 5, infra.

FIG. 9 shows the nucleotide sequence of mouse PDE5 (SEQ ID NO.: 24)

FIG. 10 shows the predicted amino acid sequence of mouse PDE5 (SEQ ID NO.: 25).

FIG. 11 shows time-dependent activation and reactivation of recombinant mouse PDE5 during preincubation with 50 μM cGMP on ice, as described in Example 12, infra.

FIG. 12 shows results indicating that recombinant PDE5 can be activated after preincubation with cGMP only under particular assay conditions at low substrate concentration, and not as a result of phosphorylation, as described in Example 12, infra.

FIG. 13 demonstrates the results of an experiment showing that PDE5 has only one phosphorylation site: the phospho-site mutant PDE5 cannot be phosphorylated by either PKG or PKA in vitro, as described in Example 12, infra. It also shows that the activation is not due to phosphorylation and therefore is due to direct activation by binding of the small molecule to the enzyme.

FIG. 14 depicts pretreatment of PDE5 with mAb/P3B2 blocks cGMP binding to PDE5, as described in Example 12, infra.

FIG. 15 depicts that the epitope for mAb/P3B2 lies in the GAF A region of PDE5, as described in Example 12, infra.

FIG. 16 depicts that PDE5 has a low intrinsic catalytic activity: blocking cGMP binding by mAb/P3B2 results in a significant reduction of PDE5 activity, as described in Example 12, infra.

FIG. 17 depicts time-dependent activation of PDE5 during its activity assay, as described in Example 12, infra.

FIG. 18 depicts that PDE5 in an activated state is more sensitive to inhibition by sildenafil, as described in Example 12, infra.

FIG. 19 depicts that PDE5 catalytic activity increases after 1-2 weeks of storage on ice accompanied by loss of its ability to cGMP stimulation, as described in Example 12, infra. Therefore, normal, stored PDE5 cannot be used for screening in an activation assay.

FIG. 20 depicts a diagram that suggests a mechanism by which PDE5 is directly activated upon cGMP binding to its GAF A domain, as described in Example 12, infra.

### DETAILED DESCRIPTION OF THE INVENTION

### DEFINITIONS

As used in this application, the following words or phrases have the meanings specified.

The term “PDE5” refers to a cGMP-binding, cGMP-specific phosphodiesterase (cGB-PDE). The terms “cGB-PDE” and “PDE5” may be used interchangeably in the application and include multiple isoforms, such as PDEA5A1, PDE5A2, PDE5A3 (Lin et al., Int. J. Impotence Res. (2002), 14: 15-24). As used herein, the term “allosteric site” refers to one or more domains of the PDE5 protein, other than the active site, that bind small molecular weight ligands, such as cGMP. In specific embodiments the “allosteric site” in PDE5 is also referred to as the “cGMP-binding domain.”

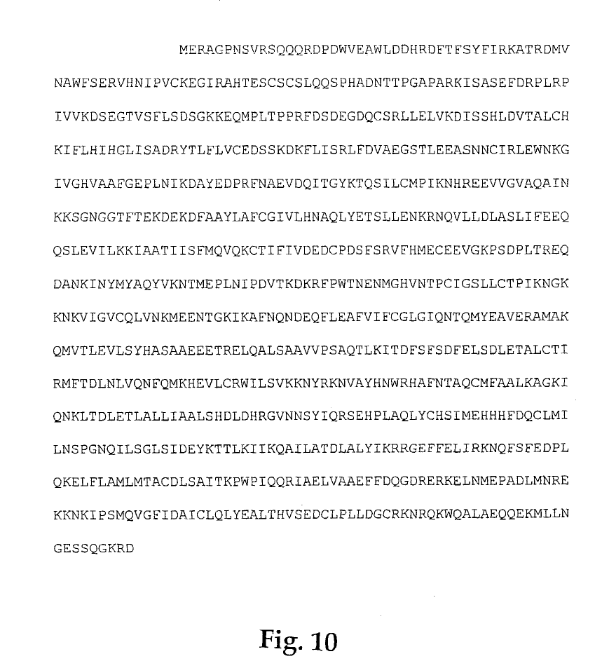

As used herein, the term “GAF domain,” refers to a highly conserved domain that binds small molecular weight ligands. The GAF domain of some PDEs is known to bind cGMP. The PDE5 protein of the invention contains two GAF domains termed the “GAF A” and “GAF B” domain. The GAF A domain of mouse PDE5 comprises amino acids 125-320 (FIG. 10) and the GAF B domain of mouse PDE5 comprises amino acids 334-525 (FIG. 10). The GAF A domain of PDE5 binds cGMP.

As used herein, the term “catalytic site” refers to a domain of a PDE5 protein that binds substrate and contributes the amino acid residues that directly participate in the catalysis reaction with the PDE5 protein, i.e., the making and breaking of chemical bonds.

As used herein, the term “modulating,” refers to a change in the activity of PDE5. For example, modulation may cause an increase or a decrease in protein amount or activity, binding characteristics, or any other biological, functional or immunological properties of PDE5.

As used herein, the term “antagonist,” or “inhibitor,” refers to a molecule which, when bound to PDE5, decreases the amount (expression) or the duration of the effect of the biological or immunological activity of the novel PDE5. Antagonists may include proteins, nucleic acids, carbohydrates, antibodies or any other molecules that decrease the amount (expression) or effect of PDE5 present in the sample. The preferred antagonist will selectively inhibit the biological activity of PDE5, not affecting any other cellular proteins.

As used herein, an agent is said to agonize or enhance PDE5 activity when the agent increases the biological activity of a PDE5 protein. The preferred agonist will selectively enhance the biological activity of PDE5.

As used herein, the term “antibody,” refers to intact molecules as well as fragments thereof, such as Fab, F(ab′)₂ and Fv fragments, which are capable of binding an epitopic determinant or epitopic determinant(s) on PDE5. The antibody can be “polyclonal,” “monoclonal,” “humanized,” or human.

As used herein, the term “biologically active” refers to a PDE5 protein having structural, regulatory, or biochemical functions of a naturally occurring PDE5 molecule. Likewise, “immunologically active” refers to the capability of the natural, recombinant, or synthetic PDE5, or any oligopeptide thereof, to induce a specific immune response in appropriate animals or cells and to bind with specific antibodies.

As used herein, the term “kinetically pure,” refers to a PDE5 protein preparation which is essentially free of all the other interfering proteins (for example, free of other cGMP hydrolyzing PDEs).

As used herein, the term “nucleic acid sequence,” refers to an oligonucleotide, nucleotide, or polynucleotide, and fragments thereof, and to DNA or RNA of genomic or synthetic origin which may be single- or double-stranded, and represents the sense or antisense strand or both.

As used herein, the term “amino acid sequence”, refers to amino acids encoding an oligopeptide, peptide, polypeptide, or protein sequence, and fragments thereof, and includes naturally occurring or synthetic molecules.

As used herein, the term “antisense,” refers to any composition containing nucleotide sequences which are complementary to a specific DNA or RNA sequence. The term “antisense strand” is used in reference to a nucleic acid strand that is complementary to the “sense” strand. Antisense molecules include nucleic acids and may be produced by any method including synthesis or transcription. Once introduced into a cell, the complementary nucleotides combine with natural sequences produced by the cell to form duplexes and block either transcription or translation of the sequences.

As used herein, the term “biological sample,” is used in its broadest sense. A biological sample is a sample suspected of containing nucleic acid encoding the PDE5 protein, or fragments thereof, or the PDE5 protein itself, or fragments thereof, having PDE5 biological activity.

The terms “specific binding,” as used herein, refers to that interaction between the PDE5 protein or a peptide thereof, and a ligand for PDE5, such as an agonist, an antibody, or an antagonist. The interaction is dependent upon the presence of a particular structure (i.e., the antigenic determinant or epitope) of the protein recognized by the binding molecule.

The term “effective” amount or a “therapeutically effective amount”, as used herein, refers to an amount of a drug or pharmacologically active agent that is nontoxic, but a sufficient amount to provide the desired effect.

As used herein the term “subject,” is used in its broadest sense. A subject includes a human, a non-human primate (e.g., monkey, baboon), a farm animal (e.g., pig, sheep, goat), a household pet (dog, cat, hamster), an experimental animal (e.g., mouse, guinea pig) and an animal in captivity or a zoo animal (e.g., panda, tiger, elephant) and the like.

As described in detail below, the present invention provides compositions and methods for modulating biological functions of PDE5.

Compositions of the Invention

In its various aspects, as described in detail below, the present invention provides agents for modulating PDE5. These agents include: proteins, peptides, antibodies, nucleic acid molecules, recombinant DNA molecules, small molecules (organic or inorganic compounds) and methods for obtaining and using the compositions of the invention, including screening and diagnostic assays, therapeutic methods, and immunological and nucleic acid-based pharmaceutical or therapeutic assays

A particular embodiment of the PDE5 proteins of the invention is mammalian PDE5. PDE5 protein of the invention has two cGMP-binding sites (also called allosteric sites) located at its N-terminal end, and a catalytic site at located at its C-terminal end (Mc Allister-Lucas et al., supra). The cGMP-binding sites have been identified as two GAF domains (GAF A and GAF B) based on their sequence homology with similar motifs in a wide group of proteins.

The cGMP binding sites of PDE5 are necessary for enhancement of serine-92 (bovine PDE5) phosphorylation by PKA or PKG in vitro (Thomas et al., (1990) J Biol Chem, 265, 14971-14978). The present invention discloses that PDE5 is directly activated upon cGMP binding to the GAF A domain. This PDE5 activation can be completely blocked by preventing GMP-binding to the GAF A domain, using an agent such as a GAF A domain-specific monoclonal antibody, indicating that molecules that bind to the GAF A domain of PDE5 may be used to modulate the biological functions of PDE5.

The invention further discloses that PDE5 can exist in at least three different conformation states: non-activated, activated by cGMP binding to the GAP A domain, and activated after storage. The non-activated low intrinsic catalytic activity state and cGMP activated states are two reversible conformational states of PDE5 with different kinetic and inhibitory properties, and these are likely the “native states”, which are present in vivo, and respond to fluctuations in cGMP levels via cGMP induced allosteric transition from the low catalytic activity state to the activated state.

However, the ability of PDE5 to be directly activated by cGMP is limited to relatively fresh preparations (less than a week after harvesting transfected cells). Longer storage results in a complete loss of the cGMP/GAF domain effect on the catalytic activity of PDE5. Thus, PDE5 activated after storage reveals an “artificial”, probably irreversible, conformational state of PDE5, suggesting that interaction between the cGMP/GAF domain and the catalytic domain has been interrupted.

Various forms of a particular PDE5 protein of the invention may be used to develop modulating agents. These include PDE5 proteins produced as a result of processes such as post-translational modification, and alternative splicing. For example, various forms of isolated PDE5 proteins may include: precursor forms, mature forms, and different mature forms of a PDE5 protein that result from post-translational events, such as, glycosylation, phosphorylation, and intramolecular cleavage.

The present invention also includes compositions and methods for modulating proteins having sequence variations from the known PDE5 protein sequences. For example, proteins having variant sequences include allelic variants, mutant variants, conservative substitution variants, and PDE5 proteins isolated from other organisms.

The present invention further encompasses mutant alleles of PDE5 that encode mutant forms of PDE5 proteins having one or more amino acid substitutions, insertions, deletions, truncations, or frame shifts, which can be used for developing modulators of PDE5. Such mutant forms of PDE5 proteins typically may not exhibit the same biological activity as wild-type proteins.

Another variant of a PDE5 protein that can be used to develop modulating agents may have amino acid sequences that differ by one or more amino acid substitutions for developing modulators of PDE5 proteins. The variant of PDE5 may have conservative amino acid changes, where a substituted amino acid has similar structural or chemical properties, such as replacement of leucine with isoleucine. Alternatively, a variant of PDE5 may have nonconservative amino acid changes.

The invention also provides peptides comprising biologically and/or immunologically active fragments of PDE5 for developing modulators of PDE5. For example, the proteins and peptides of the invention can be used to elicit antibodies that specifically bind an epitope of PDE5 protein of the invention and modulate the biological functions of PDE5. Accordingly, the PDE5 protein, or any oligopeptide thereof, is capable of inducing a specific immune response in appropriate animals or cells, and/or binding with ligands such as specific antibodies.

The various forms of PDE5 proteins described herein, may be used for developing anti-PDE5 monoclonal antibodies, and/or for screening for agents that bind PDE5 protein and modulate the biological activity of PDE5. The various PDE5 proteins for use in developing modulating agents may be generated by chemical synthesis or by recombinant methods. Recombinant methods are preferred if a high yield is desired. Recombinant methods involve expressing the cloned gene in a suitable host cell. For example, a host cell is introduced with an expression vector having a PDE5 nucleotide sequence, and then the host cell is cultured under conditions that permit production of the protein encoded by the sequence.

Antibodies Reactive Against PDE5 Proteins and Polypeptides

The invention further provides antibodies, such as polyclonal, monoclonal, chimeric, fragments, and human plus humanized antibodies, that bind to PDE5 proteins or fragments thereof, to modulate the activity of PDE5 proteins. These antibodies can be from any source, e.g., rabbit, sheep, rat, dog, cat, pig, horse, mouse and human.

As will be understood by those skilled in the art, the regions or epitopes of a PDE5 protein to which an antibody is directed may vary with the intended application. Anti-PDE5 monoclonal antibodies (mAbs) may be used as specific modulators (inhibitors or activators) of PDE5 activity. For example an anti-PDE5 mAb may bind to the catalytic domain and inhibit PDE5 activity. Alternatively, an anti-PDE5 mAb may bind to the allosteric domain and modulate (activate or inhibit) PDE5 activity.

Anti-PDE5 mAbs directed to the GAF A and GAF B domains, specifically the mAbs that bind to cGMP-binding site(s) within the GAF A or GAF B domains, are contemplated for use in modulating the activity of PDE5. Additionally, antibodies binding to phosphorylation sites of PDE5 may be useful in modulating its function and are contemplated by the invention.

The PDE5 antibodies may also be used to modulate (e.g., inhibit or activate) the biological activity of PDE5 proteins. For example, cells expressing PDE5 of the invention can be targeted, using antibodies that bind with cells expressing PDE5 proteins. This includes antibodies to the allosteric (GAF A or GAF B) domain or the catalytic domain. The antibodies that bind to a GAF domain of PDE5 and thus block the cGMP binding to GAF domain of PDE5, are particularly interesting. For example, by blocking the cGMP binding to a PDE5 GAF domain, phosphorylation of PDE5 by PKG will be blocked, leading to potential development of tolerance to NO stimulation, a condition that may affect a variety of physiological processes in cardiovascular, nervous and immune system disorders.

Additionally, the PDE5 antibodies can be used to isolate kinetically pure preparations of tissue PDE5 which would be useful for screening for agents that bind PDE5 and modulate PDE5 functions.

The methods for making antibodies, such as monoclonal antibodies, are well known in the art, using for example, hybridoma fusion techniques or by techniques that use EBV-immortalization methods. (See, e.g. Kohler and Milstein, Nature, 256:495-97 (1975); Brown et al., J. Immunol., 127 (2):539-46 (1981); Brown et al., J. Biol. Chem., 255:4980-83 (1980); Yeh et al., Proc. Natl. Acad. Sci. (USA), 76 (6):2927-31 (1976); and Yeh et al., Int. J. Cancer, 29:269-75 (1982)).

Chimeric (mouse-human e.g., humanized antibodies) or human monoclonal antibodies may be preferable to murine antibodies for some therapeutic uses, because patients treated with mouse antibodies generate human anti-mouse antibodies (Shawler et al., J. Immunol. 135:1530-35 (1985)). Chimeric mouse-human monoclonal antibodies reactive with the antigen can be produced, for example, by techniques recently developed for the production of chimeric antibodies (Oi et al., Biotechnologies 4(3):214-221 (1986); Liu et al., Proc. Nat'l. Acad. Sci. (USA) 84:3439-43 (1987)).

Novel antibodies of mouse or human origin can be also made to the antigen having the appropriate biological functions. For example, human monoclonal antibodies may be made by using the antigen, e.g. a PDE5 protein or a fragment thereof, to sensitize human lymphocytes to the antigen in vitro followed by EBV-transformation or hybridization of the antigen-sensitized lymphocytes with mouse or human lymphocytes, as described by Borrebaeck et al. (Proc. Nat'l. Acad. Sci. (USA) 85:3995-99 (1988)).

### Nucleic Acid Molecules of the Invention

The invention provides nucleic acid molecules, including variants, mutants, recombinant nucleic acids, and antisense molecules that recognize and hybridize with PDE5 nucleic acid, and modulate expression of PDE5 nucleic acid.

Antisense polynucleotides are particularly useful in regulating the expression of a PDE5 protein in cells expressing PDE5 mRNA. These include antisense oligonucleotides that block the expression of the gene encoding the PDE5 protein within cells by binding a complementary messenger RNA (mRNA) and preventing its translation (Wagner, Nature 372:332-335 (1994); and Crooke and Lebleu, Antisense Research and Applications, CRC Press, Boca Raton (1993)). Gene inhibition may be measured by determining the degradation of the target RNA. An antisense molecule corresponding to the N-terminal sequence of the gene is a particularly desirable modulating agent. The present invention includes full length and fragment antisense polynucleotides.

Antisense DNA and RNA can be prepared by methods known in the art for synthesis of RNA including chemical synthesis such as solid phase phosphoramidite chemical synthesis or in vitro and in vivo transcription of DNA sequences encoding antisense RNA molecules. The DNA sequences may be incorporated into vectors with RNA polymerase promoters. Alternatively, antisense cDNA constructs that synthesize antisense RNA constitutively or inducibly can be introduced into cell lines.

The potency of antisense oligonucleotides for inhibiting the target PDE5 peptides may be enhanced using various methods including 1) addition of polylysine (Leonetti et al., Bioconj. Biochem. 1:149-153 (1990)); 2) encapsulation into antibody targeted liposomes (Leonetti et al., Proc. Natl. Acad. Sci. USA 87:2448-2451 (1990) and Zelphati et al., Antisense Research and Development 3:323-338 (1993)); 3) nanoparticles (Rajaonarivony et al., J. Pharmaceutical Sciences 82:912-917 (1993) and Haensler and Szoka, Bioconj. Chem. 4:372-379 (1993)), 4) the use of cationic acid liposomes (Felgner et al., Proc. Natl. Acad. Sci. USA 84:7413-7417 (1987); Capaccioli et al., Biochem. Biophys. Res. Commun. 197:818-825 (1993); Boutorine and Kostina, Biochimie 75:35-41 (1993); Zhu et al., Science 261:209-211 (1992) and Wagner, Science 280:1510-1513 (1993)); and 5) Sendai virus derived liposomes (Compagnon et al., Exper. Cell Res. 200:333-338 (1992) and Morishita et al., Proc. Natl Acad. Sci. USA 90:8474-8478 (1993)), to deliver the oligonucleotides into cells. Recent techniques for enhancing delivery include the conjugation of the antisense oligonucleotides to a fusogenic peptide, e.g. derived from an influenza hemagglutinin envelope protein (Bongartz et al., Nucleic Acids Res. 22(22):4681-4688 (1994)).

The present invention further contemplates small interfering RNA (siRNA) for altering the expression of PDE5 by RNA interference (RNAi). The siRNAs, usually 21-23 nt dsRNA, have been shown to elicit strong and specific gene silencing effect in mammalian cells (Tuschl et al., Genes and Develop. (1999) 13: 3191-3197; Elbashir et al., Nature (2001) 411:494-498; Brummelkamp et al., Science (2002) 296:550-553). In cultured mammalian cells, the most efficient silencing via RNAi is achieved with siRNA duplexes composed of 21-nt sense and 21-nt antisense strands, paired in a manner so as to have a 2-nt 3′ overhang (Elbashir et al., Genes & Develop. (2001) 15:188-200). The siRNAs can be chemically synthesized using appropriately protected ribonucleotide phosphoramidites and a conventional DNA/RNA synthesizer, and introduced into an appropriate cell by transfection. The methods to synthesize and transfect siRNA are well known in the art (Elbashir et al., Nature (2001) 411:494-498). Alternatively, siRNA can be continuously produced within a cell using a plasmid based vector such as psiRNA (Invitrogen, CA) that permits a long-lasting silencing of the gene of interest (Fire et al., Nature (1998) 391: 806-811). Viral-based vectors can be used for the delivery of siRNA in various cell types to specifically alter the expression of a targeted gene (e.g., PDE5) both in vitro and in vivo (Xia et al., Naturebiotechnology (2002) 20: 1006-1010).

The present invention also contemplates recombinant nucleic acids encoding PDE5, vectors comprising nucleic acids encoding PDE5, and a host-vector system capable of producing recombinant PDE5 for use in screening and developing agents that modulate PDE5 activity. A variety of techniques and expression vector/host system may be utilized, including but not limited to bacterial, yeast, plant, isect, and mammalian expression system. These techniques and expression vector/host systems are well known in the art (Sambrook et al., Molecular Cloning (1989); Ausubel et al., Current protocols in molecular Biology, (1989) John Wiley & Sons, New York, N.Y.).

Small Molecules of the Invention

The invention further contemplates small molecules that bind PDE5 or fragments thereof, and thereby modulate the activity of PDE5 proteins. These include non-peptide organic or inorganic compounds that bind to a particular domain (e.g., allosteric or catalytic domain) of PDE5 and thereby inhibit or enhance the activity of PDE5.

Examples of small molecules that may be used to modulate PDE5 activity include, but are not limited to, sildenafil (Viagra®) (U.S. Pat. Nos. 5,955,611 and 6,066,735), IC-351 (ICOS), also referred to as tadalafil or tildenafil (Cialis™) (U.S. Pat. Nos. 6,143,746; 6, 451, 807; 6,469,016), TA1790 (Vivus), vardenafil (Bayer), and analogs thereof. Additionally, compounds including, but not limited to, the pyrazolopyrimidinones, such as those disclosed in U.S. Pat. No. 6,469,012 B1, bicyclic heterocyclic compounds such as those described in U.S. Pat. No. 6,100,270, diphenyl ether compounds such as those described in U.S. Pat. No. 6,448,293, aminoquinqzoline derivatives such as those described in U.S. Pat. No. 6,300,335 B1, and analogs thereof may be used as PDE5 modulating agents. Additional examples of small molecules include the griseolic acid derivatives, 2-phenylpurinone derivatives, phenylpyridone derivatives, fused and condensed pyrimidines, pyrimidopyrimidine derivatives, purine compounds, quinazoline compounds as disclosed in U.S. Pat. No. 4,060,615, phenylpyrimidinone derivative, imidazoquinoxalinone derivatives, other compounds disclosed in WO 96/16644, and analogs thereof. Additionally cyclic GMP analogs including, but not limited to, those described in U.S. Pat. No. 6,352,833, that bind to PDE5, may be used as modulators of PDE5 activity.

Strategies for Developing Modulators of PDE5

Developing modulators of the biological activities of specific PDEs requires differentiating PDE isozymes present in a particular assay preparation. The classical enzymological approach of isolating PDEs from natural tissue sources and studying each new isozyme is hampered by the limits of purification techniques and the inability to definitively assess whether complete resolution of an isozyme has been achieved. Another approach has been to identify assay conditions which might favor the contribution of one isozyme and minimize the contribution of others in a preparation. This has proved nearly impossible with this family of very similar isoenzymes. Still another approach has been the separation of PDEs by immunological means. This is made possible using, for example, the monoclonal antibodies described in this application.

Each of the foregoing approaches for differentiating PDE isozymes is time consuming and technically difficult. As a result many early attempts to develop selective PDE modulators have been performed with preparations containing more than one isozyme. Moreover, PDE preparations from natural tissue sources are susceptible to limited proteolysis and may contain mixtures of active proteolytic products that have different kinetic, regulatory and physiological properties than the full length PDEs.

Recombinant PDE5 polypeptide products of the invention greatly facilitate the development of new and specific PDE5 modulators. The use of recombinant enzymes for screening for modulators has many inherent advantages. The need for purification of an isozyme can be avoided by expressing it recombinantly in a host cell that lacks appreciable endogenous phosphodiesterase activity. Once a compound that modulates the activity of a PDE5 is discovered, its selectivity can be evaluated by comparing its activity on the PDE5 to its activity on other PDE isozymes. Thus, the combination of the recombinant PDE5 products of the invention with other recombinant PDE products provides a system for developing selective modulators of PDE5.

Selective modulators may include, for example, antibodies and other proteins or peptides which specifically bind to the PDE5 protein or PDE5 nucleic acid, oligonucleotides which specifically bind to PDE5 (see Patent Cooperation Treaty International Publication No. WO93/05182 published Mar. 18, 1993) which describes methods for selecting oligonucleotides which selectively bind to target biomolecules. Additionally, PDE5 nucleic acid (e.g., antisense oligonucleotides and siRNA) and other non-peptide natural or synthetic compounds which specifically bind to the PDE5 protein or PDE5 nucleic acid are contemplated as modulators of PDE5 activity.

Mutant forms of PDE5 which alter the enzymatic activity of the PDE5 or its localization in a cell are also contemplated. Crystallization of recombinant PDE5 alone and bound to a modulator, analysis of atomic structure by X-ray crystallography, and computer modeling of those structures are methods useful for designing and optimizing non-peptide selective modulators. See, for example, Erickson et al., Ann. Rep. Med. Chem., 27: 271-289 (1992), for a general review of structure-based drug design.

Targets for the development of selective modulators include, for example: (1) the regions of PDE5 which contact other proteins and/or localize the PDE5 within a cell, (2) the regions of PDE5 which bind substrate, i.e., the catalytic domain, (3) the allosteric cGMP-binding site(s) of PDE5, e.g., GAF A or GAF B domain, (4) the metal-binding regions of PDE5, (5) the phosphorylation site(s) of PDE5 and (6) the regions of PDE5 which are involved in dimerization of PDE5 subunits.

Screening for PDE5 Ligands

Another aspect of the invention relates to screening methods for identifying modulating agents and/or cellular constituents that bind to PDE5 protein (e.g., ligands) and/or modulate the biological activity of PDE5 proteins. In specific embodiments, the invention provides methods for screening for agents that bind PDE5 and/or modulate the biological activity of PDE5. In preferred embodiments, the invention provides recombinant PDE5 for screening for agents that bind PDE5 and/or modulate the biological activity of PDE5. The agents that bind with and modulate the biological activity of PDE5 may facilitate diagnosis, prevention, and treatment of PDE5-associated disorders.

The regions of PDE5 protein that may bind to a ligand include: (1) the regions of PDE5 which contact other proteins and/or localize the PDE5 within a cell, (2) the regions of PDE5 which bind substrate, i.e., the catalytic domain, (3) the allosteric cGMP-binding site(s) of PDE5, e.g., GAF A or GAF B domain, (4) the metal-binding regions of PDE5, (5) the phosphorylation site(s) of PDE5 and (6) the regions of PDE5 which are involved in dimerization of PDE5 subunits.

In one embodiment of the invention, PDE5, its allosteric domains, including GAFA and/or GAF B domain, its catalytic domain, immunogenic fragments or oligopeptides thereof, can be used for screening libraries of compounds in any of a variety of drug screening techniques. The fragment employed in such screening may be an isolated naturally occurring protein fragment, produced by recombinant means, or a chemically synthesized molecule. The fragment employed in such screening may be free in solution, affixed to a solid support, borne on a cell surface, or located intracellularly. The formation of binding complexes between PDE5 and the agent being tested may be measured by any of the techniques described infra.

In one embodiment, a screening assay is used to identify modulating agents and comprises the following: contacting a labeled PDE5 protein with a test agent or cellular extract, under conditions that allow association (e.g., binding) of the PDE5 protein with the test agent or component of the cellular extract; and determining if a complex comprising the agent or component associated with the PDE5 protein is formed.

Another technique for drug screening employs synthesizing large numbers of different small test compounds on a solid substrate, such as plastic pins or some other cell surface, and subsequently reacting these compounds with PDE5, or fragments thereof Bound PDE5 is then detected by methods well known in the art. Purified PDE5 can also be coated on to plates for use in the aforementioned drug screening technique. Alternatively, non-neutralizing antibodies can be used to capture PDE5, or a fragment thereof, and immobilize it on a solid support. The screening methods are suitable for use in high throughput screening methods.

The binding of an agent with a PDE5 protein can be assayed using a shift in the molecular weight or a change in biological activity (e.g., cGMP hydrolytic activity) of the unbound PDE5, or the expression of a reporter gene in a two-hybrid system (Fields, S. and Song, O., 1989, Nature 340:245-246).

The method used to identify whether an agent/cellular component binds to a PDE5 protein or a fragment thereof, may include a gel retardation assay. Alternatively, immunodetection and biochip (e.g., U.S. Pat. No. 4,777,019) technologies can be adopted for use with the PDE5 protein. An alternative method for identifying agents that bind with a PDE5 protein employs TLC overlay assays using glycolipid extracts from immune-type cells (K. M. Abdullah, et al., 1992 Infect. Immunol. 60:56-62). A skilled artisan can readily employ numerous art-known techniques for determining whether a particular agent binds to a PDE5 protein, or a fragment thereof, of the invention.

Alternatively, the biological activity of a PDE5 protein, as part of a complex, can be analyzed as a means for identifying agonists and antagonists of PDE5 activity. For example, a method used to isolate cellular components that bind CD22 (D. Sgroi, et al., (1993) J. Biol. Chem. 268:7011-7018; L. D. Powell, et al., (1993) J. Biol. Chem. 268:7019-7027) can be adapted to isolate cell-surface glycoproteins that bind to PDE5 proteins by contacting cell extracts with an affinity column having immobilized anti-PDE5 antibodies.

For screening assays for detecting modulation of PDE5 activity a modification of the procedure of Wells et al., Biochim. Biophys. Acta, 384:430 (1975) may be emplyed. Briefly, the assay is performed in a total volume of 200 μl containing 50 mM Tris pH 7.5, 3 mM Mg acetate, 1 mM EDTA, 50 μg/mL snake venom nucleotidase and [³H]-cGMP (Amersham). Compounds of the invention are dissolved in DMSO and incubated in the assay mixture for 30 minutes at 30° C. The assay is stopped by addition of stop buffer containing 800 μl of 10 mM Tris pH 7.5, 10 mM EDTA, 10 mM theophylline, 0.1 mM adenosine, and 0.1 mM guanosine. The mixtures are loaded on to 0.5 mL QAE Sephadex columns, and eluted with 2 mL of 0.1 M formate (pH 7.4). The eluted radioactivity is measured by scintillation counting in Optiphase Hisafe 3.

Alternatively, a microplate modification of the above PDE assay using Multiscreen plates and a vacuum manifold can be used. The assay is performed in 100 μl assay volume essentially as described above. At the end of the incubation, the total volume of the assay is loaded on a QAE Sephadex microcolumn plate by filtration. Free radioactivity is eluted with 200 μl of water from which 50 μl aliquots are analyzed by scintillation counting as described above.

Alternatively or consecutively, an intact cell screening assay using a reporter gene responsive to cGMP levels is used. This system uses engineered cells containing a cGMP activated Ca²⁺ channel that responds fluorometrically to Ca²⁺ influx (T. Rech et al., (2001) J. Gen. Phys. 118: 63-78). In this system, a change in Ca²⁺ influx in response to a stimulus reflects a change in cGMP level, which is used to assess the interaction of PDE5 and a modulator. In this assay, an inhibitor of PDE5 would increase cGMP content and therefore result in increased Ca²⁺ fluorescence signal. On the other hand, an agonist screen assay would look for a decrease in cGMP level, and therefore detect a decreased Ca²⁺ fluorescence signal.

Another embodiment of the assays of the invention includes screening agents and cellular constituents that bind to a PDE5 protein using a yeast two-hybrid system (Fields, S. and Song, O., supra) or using a binding-capture assay (Harlow, supra). Generally, the yeast two-hybrid system is performed in a yeast host cell carrying a reporter gene, and is based on the modular nature of the GAL transcription factor, which has a DNA binding domain and a transcriptional activation domain. The two-hybrid system relies on the physical interaction between a recombinant protein that comprises the DNA binding domain and another recombinant protein that comprises the transcriptional activation domain to reconstitute the transcriptional activity of the modular transcription factor, thereby causing expression of the reporter gene. Either of the recombinant proteins used in the two-hybrid system can be constructed to include the PDE5-encoding sequence to screen for binding partners of PDE5. The yeast two-hybrid system can be used to screen cDNA expression libraries (G. J. Hannon, et al., (1993) Genes and Dev. 7: 2378-2391), and random aptmer libraries (J. P. Manfredi, et al., (1996) Molec. And Cell. Biol. 16: 4700-4709) or semi-random (M. Yang, et al., (1995) Nucleic Acids Res. 23: 1152-1156) aptmer libraries for PDE5 ligands.

The PDE5 proteins which are used in the screening assays described herein, include, but are not limited to, an isolated PDE5 protein, a fragment of a PDE5 protein, a cell that has been altered to express a PDE5 protein, or a fraction of a cell that has been altered to express a PDE5 protein.

The candidate agents to be tested for binding with PDE5 proteins and/or modulating the activity of PDE5 proteins can be, as examples, peptides, small molecules, and vitamin derivatives, as well as carbohydrates. A skilled artisan can readily recognize that there is no limit as to the structural nature of the agents tested for binding to PDE5 proteins. Candidate agents that are tested for binding with PDE5 proteins and/or modulating the activity of PDE5 proteins can be randomly selected or rationally selected. As used herein, an agent is said to be randomly selected when the agent is chosen randomly without considering the specific sequences of the PDE5 protein. Examples of randomly selected agents are members of a chemical library, a peptide combinatorial library, a growth broth of an organism, or plant extract.

As used herein, an agent is said to be rationally selected when the agent is chosen on a nonrandom basis that is based on the sequence of the PDE5 target site, and/or its conformation, in connection with the agent's action. Agents can be rationally selected by utilizing the peptide sequences that make up the PDE5 protein.

Additionally, competitive drug screening assays in which neutralizing antibodies capable of binding PDE5 specifically compete with a test compound for binding PDE5. In this assay, the antibodies can be used to detect, for molecules that bind to PDE5.

The cellular extracts to be tested for binding with PDE5 proteins and/or modulating the activity of PDE5 proteins can be, as examples, aqueous extracts of cells or tissues, organic extracts of cells or tissues or partially purified cellular fractions. A skilled artisan can readily recognize that there is no limit as to the source of the cellular extracts used in the screening methods of the present invention.

Uses of the Compositions of the Invention

The compositions of the invention may be used in methods for modulating the biological functions of PDE5, and thereby treating or preventing disorders associated with PDE5 activity. For example the compositions of the invention may bind to the catalytic domain of PDE5 and inhibit PDE5 activity. Alternatively, the compositions of the invention may bind to an allosteric domain of PDE5 and modulate (activate or inhibit) PDE5 activity. Specific embodiments of the invention provide methods for regulation of PDE5 activity via direct activation of PDE5 upon cGMP-binding to the GAF A or GAF B domain. Specifically, the invention provides agents targeted to the cGMP-binding domain (GAF A or GAF B) of PDE5.

In one embodiment, the invention provides agents (cGMP antagonists), that block cGMP binding to the GAF (GAF A or GAF B) domain of a PDE5. The cGMP antagonists targeted to, for example, the GAF A or GAF B domain of PDE5 will create a new class of PDE5 specific inhibitors with greater isozyme selectivity. In addition, by blocking cGMP binding, phosphorylation of PDE5 by PKG is prevented, otherwise leading to the potential development of tolerance to NO stimulation that may affect a variety of physiological processes in the cardiovascular, nervous and immune systems.

In another embodiment, the invention provides agonists for cGMP binding sites of PDE5 that lower intracellular cGMP concentration. Such an agent can be used for example, in treatment of conditions characterized by neuronal excitotoxicity or ischemia.

The compositions and methods for modulation of PDE5 activity may be useful in treating or preventing disorders associated with the presence, the deficiency, altered levels, or altered activity of PDE5 proteins in a subject. The disorders which are associated with PDE5 activity include, but are not limited to, male erectile dysfunction (MED), female sexual dysfunction (FSD), male and female fertility, premature labor, dysmenorrhoea, benign prostatic hyperplasia (BPH), bladder outlet obstruction, incontinence, stable, unstable and variant (Prinzmetal) angina, hypertension, pulmonary hypertension, pulmonary arterial hypertension, chronic obstructive pulmonary disease, acute respiratory distress syndrome, malignant hypertension, pheochromocytoma, congestive heart failure, acute renal failure, chronic renal failure, atherosclerosis, conditions of reduced blood vessel patency (e.g. post PTCA), a peripheral vascular disease, a vascular disorder, thrombocythemia, an inflammatory disease, myocardial infarction, stroke, bronchitis, chronic asthma, allergic asthma, allergic rhinitis, glaucoma, postpercutaneous transluminal coronary or carotid angioplasty, post-bypass surgery graft stenosis, osteoporosis, preterm labor, benign prostatic hypertrophy, irritable bowel syndrome, peptic ulcer, diseases characterized by disorders of gut motility, appetite, depression, anxiety, motor function, memory, immune function, inflammation, autoimmune disease.

The composition and methods of the invention may also be used for treating conditions where the subject responds positively to vasodilatory drugs (Michelakis, et al., Circulation (2002)105: 2398-2403). These conditions include, but are not limited to, pulmonary arterial hypertension, congenital heart defects, and pulmonary fibrosis in autoimmune disorders, which stiffen the lung tissues and blood vessels.

Additionally, the agents (especially, activators of PDE5) and methods of the invention may be used for conditions including, but not limited to, amelioration of reperfusion injury, stroke, sepsis, hypotension, reversal of nitrovasodilator overdose, including overdose of Viagra.

Suitable carriers for pharmaceutical compositions of the invention include any material which when combined with a composition of the invention retains the molecule's activity and is non-reactive with the subject's immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets, powder, syrups, and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres.

For therapeutic uses, an effective amount of the compositions of the invention can be formulated for a variety of modes of administration known in the art, including parenteral, for example intravenous, intraperitoneal, intramuscular, intradermal, subcutaneous, and epidermal, or oral, controlled release patch, continuous infusion, or applied to mucosal surfaces, e.g. by intranasal administration using inhalation of aerosol suspensions, gene therapy, liposomes, and by implanting to muscle or other tissue in the subject. Biodegradable polymers, including hydrogels (Vogelson, Modern Drug Discov. (2001) 4; Dorski, et al., Polym. Mater. Sci. Eng. Proc. (1997) 76: 281), tailored for precise delivery of the composition of the invention, are also contemplated. Suppositories and topical preparations may also be used. For oral administration, the compounds can be formulated into conventional oral administration forms such as capsules, tablets, and tonics. For topical administration, the compounds of the invention can be formulated into ointments, salves, gels, or creams as generally known in the art. Techniques and formulations generally may be found in Remmington's Pharmaceutical Sciences, Meade Publishing Co., Easton, Pa.

The most effective mode of administration and dosage regimen for the compositions of the invention depend on factors including the type and severity of disease, the subject's health, previous medical history, age, weight, height, sex and response to treatment, as well as the judgment of the treating physician. Therefore, the amount of composition to be administered, as well as the number and timing of subsequent administrations, are determined by a medical professional conducting therapy based on the response of the individual subject. Initially, such parameters are readily determined by skilled practitioners using appropriate testing in animal models for safety and efficacy, and in human subjects during clinical trials of candidate therapeutic inhibitor/actovator formulations. Accordingly, the dosages of the compositions of the invention for treatment of a subject are to be titrated to the individual subject. For example, the interrelationship of dosages for animals of various sizes and species and humans based on mg/m² of surface area is described by Freireich et al., Cancer Chemother. Rep. 50(4):219-244 (1966). The “effective dose” can be determined by procedures known in the art. For administration to mammals, and particularly humans, a typical daily dosage level of an active agent will be from 0.01 mg/kg of body weight to 100 mg/kg of body weight, typically around 1 mg/kg to 10 mg/kg body weight. The above dosages are exemplary of the average case. There can, of course, be individual instances where higher or lower dosage ranges are merited, and such are within the scope of this invention.

### EXAMPLES

The following examples are presented to illustrate the present invention and to assist one of ordinary skill in making and using the same. The methodology and results may vary depending on the intended goal of treatment and the procedures employed. The examples are not intended in any way to otherwise limit the scope of the invention.

### Example 1

The following example describes the isolation of a bovine cGB-PDE cDNA fragment by PCR and subsequent isolation of a full length cGB-PDE cDNA using the PCR fragment as a probe.

The polymerase chain reaction (PCR) was utilized to isolate a cDNA fragment encoding a portion of cGB-PDE from bovine lung first strand cDNA. Fully degenerate sense and antisense PCR primers were designed based on the partial cGB-PDE amino acid sequence described in Thomas et al. (J. Biol. Chem., 265: 14971-14978 (1990)), and novel partial amino acid sequence information.

A. Purification of cGB-PDE Protein

cGB-PDE was purified as described in Thomas et al., supra, or by a modification of that method as described below.

Fresh bovine lungs (5-10 kg) were obtained from a slaughterhouse and immediately placed on ice. The tissue was ground and combined with cold PEM buffer (20 mM sodium phosphate, pH 6.8, containing 2 mM EDTA and 25 mM beta-mercaptoethanol). After homogenization and centrifugation, the resulting supernatant was incubated with 4-7 liters of DEAE-cellulose (Whatman, UK) for 3-4 hours. The DEAE slurry was then filtered under vacuum and rinsed with multiple volumes of cold PEM. The resin was poured into a glass column and washed with three to four volumes of PEM. The protein was eluted with 100 mM NaCl in PEM and twelve 1-liter fractions were collected. Fractions were assayed for IBMX-stimulated cGMP binding and cGMP phosphodiesterase activities by standard procedures described in Thomas et al., supra. Appropriate fractions were pooled, diluted 2-fold with cold, deionized water and subjected to Blue Sepharose™ CL-6B (Pharmacia LKB Biotechnology Inc., Piscataway, N.J.) chromatography. Zinc chelate affinity adsorbent chromatography was then performed using either an agarose or Sepharose-based gel matrix. The resulting protein pool from the zinc chelation step treated as described in the Thomas et al., supra, or was subjected to a modified purification procedure.

As described in Thomas et al., supra, the protein pool was applied in multiple loads to an HPLC Bio-Sil TSK-545 DEAE column (150×21.5 mm) (BioRad Laboratories, Hercules, Calif.) equilibrated in PEM at 4° C. After an equilibration period, a 120-ml wash of 50 mM NaCl in PEM was followed by a 120-ml linear gradient (50-200 mM NaCl in PEM) elution at a flow rate of 2 ml/minute. Appropriate fractions were pooled and concentrated in dialysis tubing against Sephadex G-200 (Boehringer Mannheim Biochemicals, UK) to a final volume of 1.5 ml. The concentrated cGB-PDE pool was applied to an HPLC gel filtration column (Bio-Sil TSK-250, 500×21.5 mm) equilibrated in 100 mM sodium phosphate, pH 6.8, 2 mM EDTA, 25 mM β-mercaptoethanol and eluted with a flow rate of 2 ml/minute at 4° C.

If the modified, less cumbersome procedure was performed, the protein pool was dialyzed against PEM for 2 hours and loaded onto a 10 ml preparative DEAE Sephacel column (Pharmacia) equilibrated in PEM buffer. The protein was eluted batchwise with 0.5M NaCl in PEM, resulting in an approximately 10-15 fold concentration of protein. The concentrated protein sample was loaded onto an 800 ml (2.5 cm×154 cm) Sephacryl S400 gel filtration column (Boehringer) equilibrated in 0.1 M NaCl in PEM, and eluted at a flow rate of 1.7 ml/minute.

The purity of the protein was assessed by Coomassie staining after sodium dodecyl sulfate-polyacrylamide gel electrophoresis (SDS-PAGE). Approximately 0.5-3.0 mg of pure cGB-PDE was obtained per 10 kg bovine lung.

Rabbit polyclonal antibodies specific for the purified bovine cGB-PDE were generated by standard procedures.

B. Amino Acid Sequencing of cGB-PDE

cGB-PDE phosphorylated with [³²P]ATP and was then digested with protease to yield ³²P-labelled phosphopeptides. Approximately 100 μg of purified cGB-PDE was phosphorylated in a reaction mixture containing 9mM MgCl₂, 9 μM [³²P]ATP, 10 μM cGMP, and 4.2 μg purified bovine catalytic subunit of cAMP-dependent protein kinase (cAK) in a final volume of 900 μl. Catalytic subunit of cAK was prepared according to the method of Flockhart et al., pp. 209-215 in Marangos et al., Brain Receptor Methodologies, Part A, Academic Press, Orlando, Fla. (1984). The reaction was incubated for 30 minutes at 30° C., and stopped by addition of 60 μl of 200 mM EDTA.

To obtain a first peptide sequence from cGB-PDE, 3.7 μl of a 1 mg/ml solution of a α-chymotrypsin in KPE buffer (10 mM potassium phosphate, pH 6.8, with 2 mM EDTA) was added to 100 μg purified, phosphorylated cGB-PDE and the mixture was incubated for 30 minutes at 30° C. Proteolysis was stopped by addition of 50 μl of 10% SDS and 25 μl of β-mercaptoethanol. The sample was boiled until the volume was reduced to less than 400 μl, and was loaded onto an 8% preparative SDS-polyacrylamide gel and subjected to electrophoresis at 50 mAmps. The separated digestion products were electroblotted onto Immobilon polyvinylidene difluoride (Millipore, Bedford, Mass.), according to the method of Matsudaira, J. Biol. Chem, 262: 10035-10038 (1987). Transferred protein was identified by Coomassie Blue staining, and a 50 kDa band was excised from the membrane for automated gas-phase amino acid sequencing. The sequence of the peptide obtained by the α-chymotryptic digestion procedure is set out below as SEQ ID NO: 1.

| | |
|---|---|
| REXDANRINYMYAQYVKNTM | (SEQ ID NO: 1) |

A second sequence was obtained from a cGB-PDE peptide fragment generated by V8 proteolysis. Approximately 200 μg of purified cGB-PDE was added to 10 mM MgCl₂, 10 μM [³²P]ATP, 100 EM cGMP, and 1 μg/ml purified catalytic subunit of cAK in a final volume of 1.4 ml. The reaction was incubated for 30 minutes at 30° C., and was terminated by the addition of 160 μl of 0.2M EDTA. Next, 9 μl of 1 mg/ml Staphylococcal aureus V8 protease (International Chemical Nuclear Biomedicals, Costa Mesa, Calif.) diluted in KPE was added, followed by a 15 minute incubation at 30° C. Proteolysis was stopped by addition of 88 μl of 10% SDS and 45 μl β-mercaptoethanol. The digestion products were separated by electrophoresis on a preparative 10% SDS-polyacrylamide gel run at 25 mAmps for 4.5 hours. Proteins were electroblotted and stained as described above. A 28 kDa protein band was excised from the membrane and subjected to automated gas-phase amino acid sequencing. The sequence obtained is set out below as SEQ ID NO: 2.

| | |
|---|---|
| QSLAAAVVP | (SEQ ID NO: 2) |

C. PCR Amplification of Bovine cDNA

The partial amino acid sequences utilized to design primers (SEQ ID NO: 3, below, and amino acids 9-20 of SEQ ID NO: 1) and the sequences of the corresponding PCR primers (in IUPAC nomenclature) are set below wherein SEQ ID NO: 3 is the sequence reported in Thomas et al., supra.

| | |
|---|---|
| F D N D E G E Q | (SEQ ID NO: 3) |
| 5′ TTY GAY AAY GAY GAR GGN GAR CA 3′ | (SEQ ID NO: 4) |
| 3′ AAR CTR TTR CTR CTY CCN CTY GT 5′ | (SEQ ID NO: 5) |
| N Y M Y A Q Y V K N T M | (SEQ ID NO: 1, Amino acids 9-20) |
| 5′ AAY TAY ATG TAY GCN CAR TAY GT 3′ | (SEQ ID NO: 6) |
| 3′ TTR ATR TAC ATR CGN GTY ATR CA 5′ | (SEQ ID NO: 7) |
| 3′ TTR ATR TAC ATR CGN GTY ATR CAN TTY TTR TGN TAC 5 | (SEQ ID NO: 8) |

The sense and antisense primers, synthesized using an Applied Biosystems Model 380A DNA Synthesizer (Foster City, Calif.), were used in all possible combinations to amplify cGB-PDE-specific sequences from bovine lung first strand cDNA as described below.

After ethanol precipitation, pairs of oligonucleotides were combined (SEQ ID NO: 4 or 5 combined with SEQ ID NOs: 6, 7 or 8) at 400 nM each in a PCR reaction. The reaction was run using 50 ng first strand bovine lung cDNA (generated using AMV reverse transcriptase and random primers on oligo dT selected bovine lung mRNA), 200 μM dNTPs, and 2 units of Taq polymerase. The initial denaturation step was carried out at 94° C. for 5 minutes, followed by 30 cycles of a 1 minute denaturation step at 94° C., a two minute annealing step at 50° C., and a 2 minute extension step at 72° C. PCR was performed using a Hybaid Thermal Reactor (ENK Scientific Products, Saratoga, Calif.) and products were separated by gel electrophoresis on a 1% low melting point agarose gel run in 40 mM Tris-acetate, 2 mM EDTA. A weak band of about 800-840 bp was seen with the primers set out in SEQ ID NOs: 4 and 7 and with primers set out in SEQ ID NOs: 4 and 8. None of the other primer pairs yielded visible bands. The PCR product generated by amplification with the primers set out in SEQ ID NOs: 4 and 7 was isolated using the Gene Cleans™ (Bio101, La Jolla, Calif.) DNA purification kit according to the manufacturer's protocol. The PCR product (20 ng) was ligated into 200 ng of linearized pBluescript KS(+) (Stratagene, La Jolla, Calif.), and the resulting plasmid construct was used to transform E. coli XL1 Blue cells (Stratagene Cloning Systems, La Jolla, Calif.). Putative transformation positives were screened by sequencing. The sequences obtained were not homologous to any known PDE sequence or to the known partial cGB-PDE sequences.

PCR was performed again on bovine lung first strand cDNA using the primers set out in SEQ ID NOs: 4 and 7. A clone containing a 0.8 Kb insert with a single large open reading frame was identified. The open reading frame encoded a polypeptide that included the amino acids KNTM (amino acids 17-20 of SEQ ID NO: 1 which were not utilized to design the primer sequence which is set out in SEQ ID NO: 7) and that possessed a high degree of homology to the deduced amino acid sequences of the cGs-, ROS- and COS-PDEs. The clone identified corresponds to nucleotides 489-1312 of SEQ ID NO: 9.

D. Construction and Hybridization Screening of a Bovine cDNA Library

In order to obtain a cDNA encoding a full-length cGB-PDE, a bovine lung cDNA library was screened using the ³²P-labelled PCR-generated cDNA insert as a probe.

Polyadenylated RNA was prepared from bovine lung as described Sonnenburg et al., J. Biol. Chem., 266: 17655-17661 (1991). First strand cDNA was synthesized using AMV reverse transcriptase (Life Sciences, St. Petersburg, Fla.) with random hexanucleotide primers as described in Ausubel et al., Current Protocols in Molecular Biology, John Wiley & Sons, New York (1987). Second strand cDNA was synthesized using E. coli DNA polymerase I in the presence of E. coli DNA ligase and E. coli RNAse H. Selection of cDNAs larger than 500 bp was performed by Sepharose™ CL-4B (Millipore) chromatography. EcoRI adaptors (Promega, Madison, Wis.) were ligated to the cDNA using T4 DNA ligase. Following heat inactivation of the ligase, the cDNA was phosphorylated using T4 polynucleotide kinase. Unligated adaptors were removed by Sepharose™ CL-4B chromatography (Pharmacia, Piscataway, N.J.). The cDNA was ligated into EcoRI-digested, dephosphorylated lambda Zap™ II arms (Stratagene) and packaged with Gigapack™ Gold (Stratagene) extracts according to the manufacturer's protocol. The titer of the unamplified library was 9.9×10⁵ with 18% nonrecombinants. The library was amplified by plating 50,000 plaque forming units (pfu) on to twenty 150 mm plates, resulting in a final titer of 5.95×10⁶ pfu/ml with 21% nonrecombinants.

The library was plated on twenty-four 150 mm plates at 50,000 pfu/plate, and screened with the ³²P-labelled cDNA clone. The probe was prepared using the method of Feinberg et al., Anal. Biochem., 137: 266-267 (1984), and the ³²P-labelled DNA was purified using Elutip-D™ columns (Schleicher and Schuell Inc., Keene, N.H.) using the manufacturer's protocol. Plaque-lifts were performed using 15 cm nitrocellulose filters. Following denaturation and neutrlization, DNA was fixed onto the filters by baking at 80° C. for 2 hours. Hybridization was carried out at 42° C. overnight in a solution containing 50% formamide, 5×SSC (0.75M NaCl, 0.75M sodium citrate, pH 7), 25 mM sodium phosphate (pH 7.0), 2× Denhardt's solution, 10% dextran sulfate, 90 μg/ml yeast tRNA, and approximately 10⁶ Cpm/ml ³²P-labelled probe (5×10⁸ cpm/μg). The filters were washed twice in 0.1×SSC, 0.1% SDS at room temperature for 15 minutes per wash, followed by a single 20 minute wash in 0.1×SSC, 1% SDS at 45° C. The filters were then exposed to X-ray film at −70° C. for several days.

Plaques that hybridized with the labelled probe were purified by several rounds of replating and rescreening. Insert cDNAs were subcloned into the pBluescript SK(−) vector (Stratagene) by the in vivo excision method described by the manufacturer's protocol. Southern blots were performed in order to verify that the rescued cDNA hybridized to the PCR probe. Putative cGB-PDE cDNAs were sequenced using Sequenase™ Version 2.0 (United States Biochemical Corporation, Cleveland, Ohio) or TaqTrack™ kits (Promega).

Three distinct eDNA clones designated cGB-2, cGB-8 and cGB-10 were isolated. The DNA and deduced amino acid sequences of clone cGB-8 are set out in SEQ ID NOs: 9 and 10. The DNA sequence downstream of nucleotide 2686 may represent a cloning artifact. The DNA sequence of cGB-10 is identical to the sequence of cGB-8 with the exception of one nucleotide. The DNA sequence of clone cGB-2 diverges from that of clone cGB-8 5′ to nucleotide 219 of clone cgb-8 (see SEQ ID NO: 9) and could encode a protein with a different amino terminus.

The cGB-8 cDNA clone is 4474 bp in length and contains a large open reading frame of 2625 bp. The triplet ATG at position 99-101 in the nucleotide sequence is predicted to be the translation initiation site of the cGB-PDE gene because it is preceded by an in-frame stop codon and the surrounding bases are compatible with the Kozak consensus initiation site for eucaryotic mRNAs. The stop codon TAG is located at positions 2724-2726, and is followed by 1748 bp of 3′ untranslated sequence. The sequence of cGB-8 does not contain a transcription termination consensus sequence, therefore the clone may not represent the entire 3′ untranslated region of the corresponding mRNA.

The open reading frame of the cGB-8 cDNA encodes an 875 amino acid polypeptide with a calculated molecular mass of 99.5 kD. This calculated molecular mass is only slightly larger than the reported molecular mass of purified cGB-PDE, estimated by SDS-PAGE analysis to be approximately 93 kDa. The deduced amino acid sequence of cGB-8 corresponded exactly to all peptide sequences obtained from purified bovine lung cGB-PDE providing strong evidence that cGB-8 encodes cGB-PDE.

### Example 2

The following example presents an analysis of the relationship of the bovine cGB-PDE amino acid sequence to sequences reported for various other PDEs.

A search of the SWISS-PROT and GEnEmbl data banks (Release of February, 1992) conducted using the FASTA program supplied with the Genetics Computer Group (GCG) Software Package (Madison, Wis.) revealed that only DNA and amino acid sequences reported for other PDEs displayed significant similarity to the DNA and deduced amino acid of clone cGB-8.

Pairwise comparisons of the cGB-PDE deduced amino acid sequence with the sequences of eight other PDEs were conducted using the ALIGN [Dayhoff et al, Methods Enzymol., 92: 524-545 (1983)] and BESTFTT (Wilbur et al., Proc. Natl. Acad. Sci. USA, 80: 726-730 (1983)) programs. Like all mammalian phosphodiesterases sequenced to date, cGB-PDE contains a conserved catalytic domain sequence of approximately 250 amino acids in the carboxyl-terminal half of the protein that is thought to be essential for catalytic activity. This segment comprises amino acids 578-812 of SEQ ID NO: 9 and exhibits sequence conservation with the corresponding regions of other PDEs. Table 1 below sets out the specific identity values obtained in pairwise comparisons of other PDEs with amino acids 578-812 of cGB-PDE, wherein “ratdunce” is the rat cAMP-specific PDE; “61 kCaM” is the bovine 61 kDa calcium/calmodulin-dependent PDE; “63 kCaM” is the bovine 63 kDa calcium/calmodulin-dependent PDE; “drosdunce” is the drosophila cAMP-specific dunce PDE; “ROS-α” is the bovine ROS-PDE α-subunit; “ROS-β” is the bovine ROS-PDE β-subunit; “COS-α” is the bovine COS-PDE α′ subunit; and “cGs” is the bovine cGs-PDE (612-844).

TABLE 1

| Phosphodiesterase | Catalytic Domain Residues | % Identity |
|---|---|---|
| Ratdunce | 77-316 | 31 |
| 61 kCaM | 193-422 | 29 |
| 63 kcam | 195-424 | 29 |
| drosdunce | 1-239 | 28 |
| ROS-α | 535-778 | 45 |
| ROS-β | 533-776 | 46 |
| COS-α′ | 533-776 | 48 |
| cGs | 612-844 | 40 |

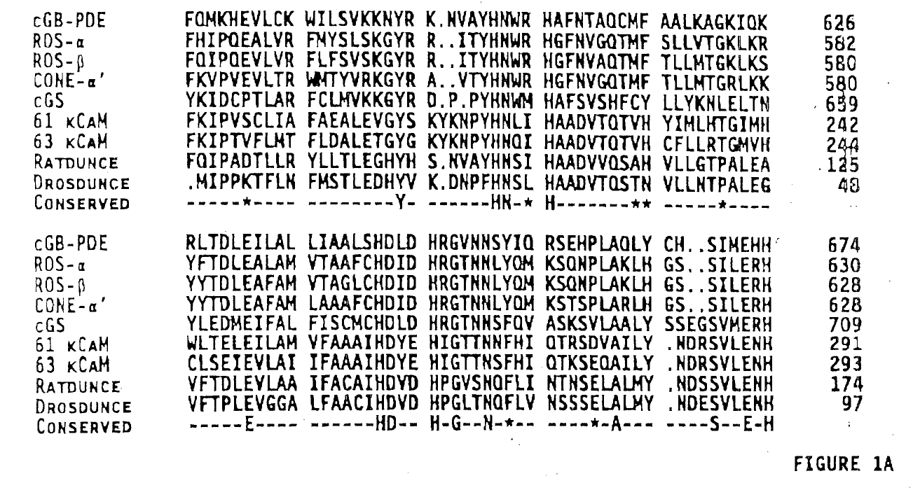

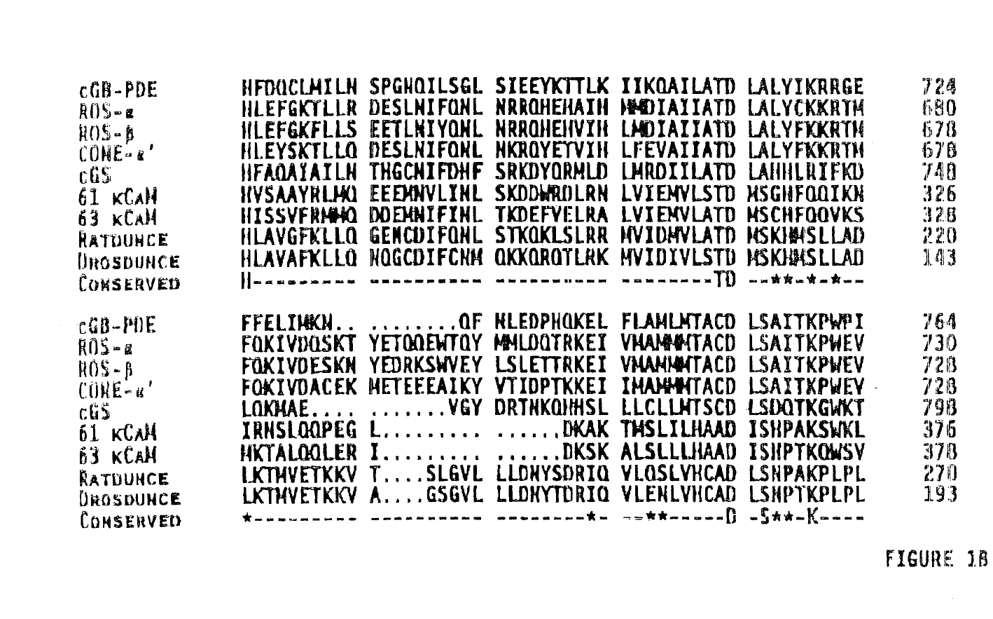

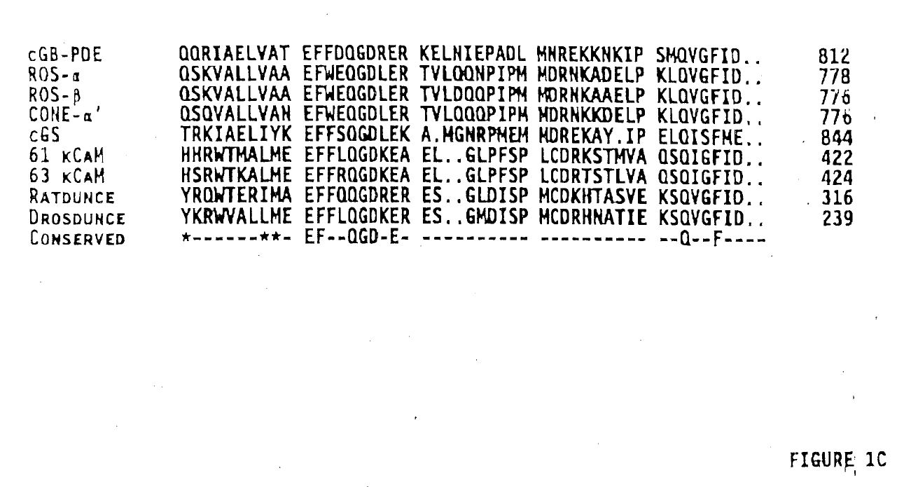

Multiple sequence alignments were performed using the Progressive Alignment Algorithm (Feng et al., Methods Enzymol., 183: 375-387 (1990)) implemented in the PILEUP program (GCG Software). FIG. 1A to 1C shows a multiple sequence alignment of the proposed catalytic domain of cGB-PDE with the all the corresponding regions of the PDEs of Table 1. Twenty-eight residues (see residues indicated by one letter amino acid abbreviations in the “conserved” line on FIG. 1A to 1C) are invariant among the isoenzymes including several conserved histidine residues predicted to play a functional role in catalysis. See Charbonneau et al., (Proc. Natl. Acad. Sci. USA, 87: 288-292 (1990)). The catalytic domain of cGB-PDE more closely resembles the catalytic domains of the ROS-PDEs and COS-PDEs than the corresponding regions of other PDE isoenzymes. There are several conserved regions among the photoreceptor PDE and cGB-PDE that are not shared by other PDEs. Amino acid positions in these regions that are invariant in the photoreceptor PDE and cGB-PDE sequences are indicated by stars in the “conserved” line of FIG. 1A to 1C. Regions of homology among cGB-PDE and the ROS- and COS-PDEs may serve important roles in conferring specificity for cGMP hydrolysis relative to cAMP hydrolysis or for sensitivity to specific pharmacological agents.

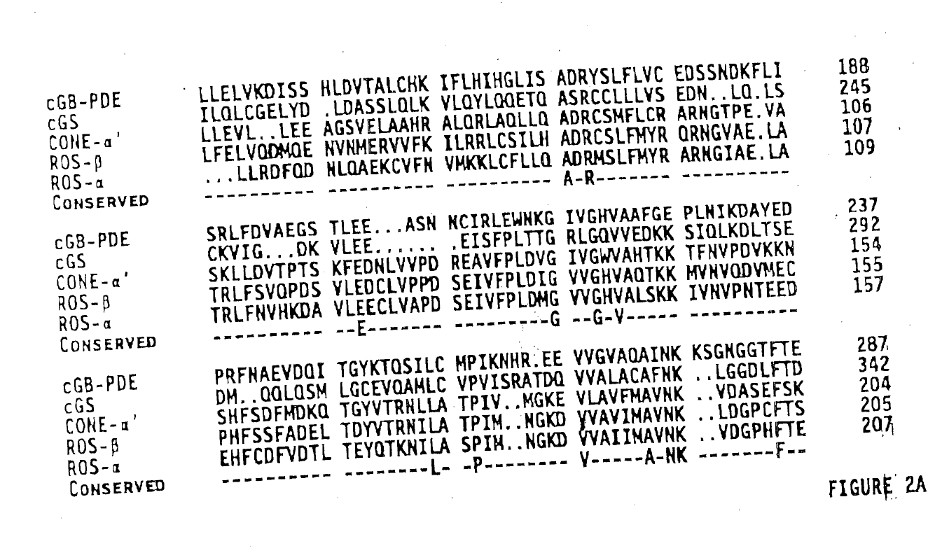

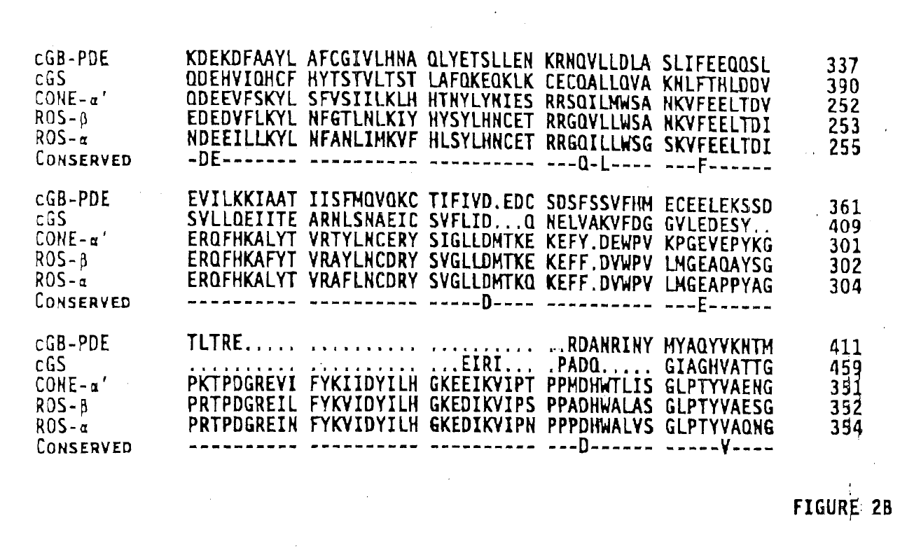

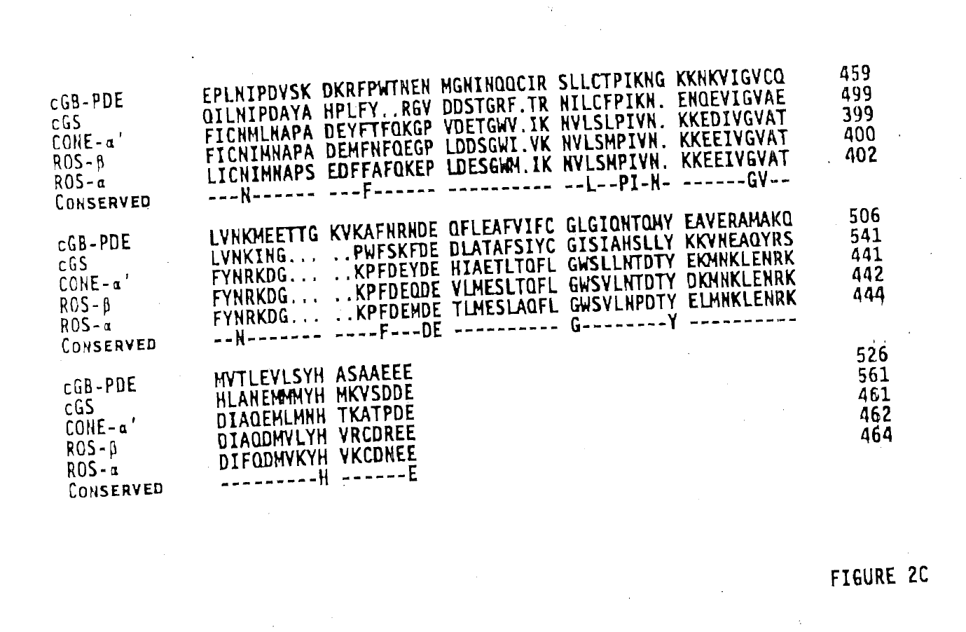

Sequence similarity between cGB-PDE, cGs-PDE and the photoreceptor PDEs, is not limited to the conserved catalytic domain but also includes the noncatalytic cGMP binding domain in the amino-terminal half of the protein. Optimization of the alignment between cGB-PDE, cGs-PDE and the photoreceptor PDEs indicates that an amino-terminal conserved segment may exist including amino acids 142-526 of SEQ ID NO: 9. Pairwise analysis of the sequence of the proposed cGMP-binding domain of cGB-PDE with the corresponding regions of the photoreceptor PDEs and cGs-PDE revealed 26-28% sequence identity. Multiple sequence alignment of the proposed cGMP-binding domains with the cGMP-binding PDEs is shown in FIG. 2A to 2C wherein abbreviations are the same as indicated for Table 1. Thirty-eight positions in this non-catalytic domain appear to be invariant among all cGMP-binding PDEs (see positions indicated by one letter amino acid abbreviations in the “conserved” line of FIG. 2A to 2C).

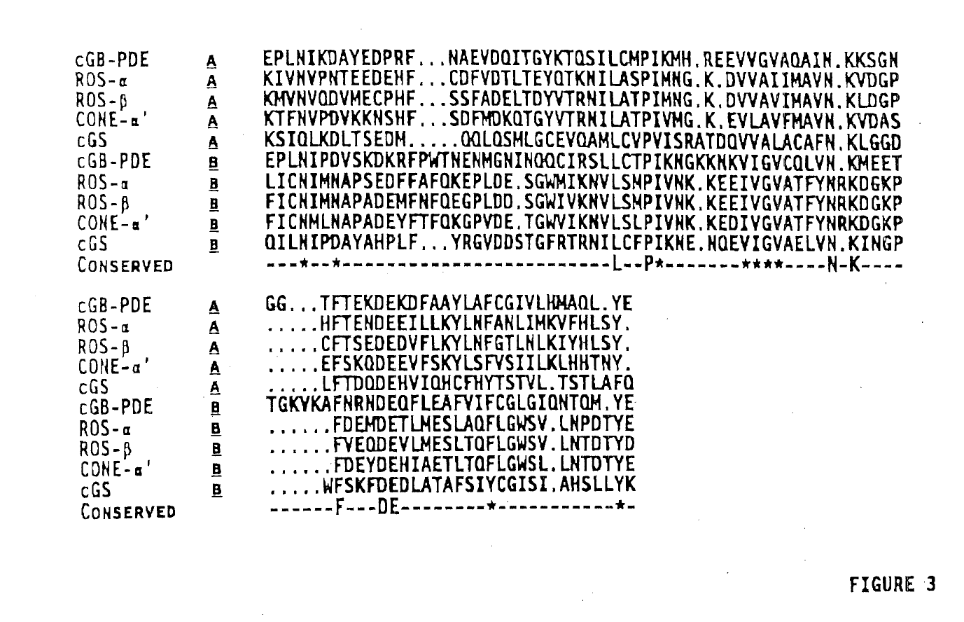

The cGMP-binding domain of the cGMP-binding PDEs contains internally homologous repeats which may form two similar but distinct inter- or intra-subunit cGMP-binding sites. FIG. 3 shows a multiple sequence alignment of the repeats a (corresponding to amino acids 228-311 of cGB-PDE) and b (corresponding to amino acids 410-500 of cGB-PDE) of the cGMP-binding PDEs. Seven residues are invariant in each A and B regions (see residues indicated by one letter amino acid abbreviations in the “conserved” line of FIG. 3). Residues that are chemically conserved in the A and B regions are indicated by stars in the “conserved” line of FIG. 3. cGMP analog studies of cGB-PDE support the existence of a hydrogen bond between the cyclic nucleotide binding site on cGB-PDE and the 2′OH of cGMP.

Three regions of cGB-PDE have no significant sequence similarity to other PDE isoenzymes. These regions include the sequence flanking the carboxyl-terminal end of the catalytic domain (amino acids 812-875), the sequence separating the cGMP-binding and catalytic domains (amino acids 527-577) and the amino-terminal sequence spanning amino acids 1-141. The site (the serine at position 92 of SEQ ID NO: 10) of phosphorylation of cGB-PDE by cGK is located in this amino-terminal region of sequence. Binding of cGMP to the allosteric site on cGB-PDE is required for its phosphorylation.

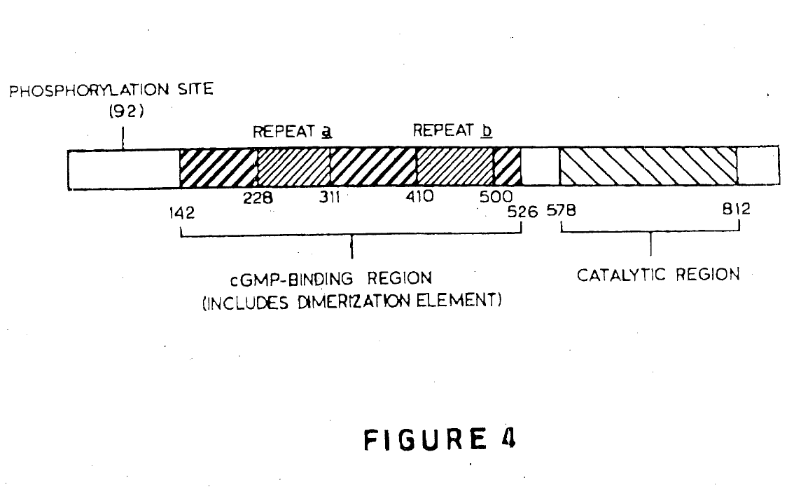

A proposed domain structure of cGB-PDE based on the foregoing comparisons with other PDE isoenzymes is presented in FIG. 4. This domain structure is supported by the biochemical studies of cGB-PDE purified from bovine lung.

### Example 3

This Example describes the presence of cGB-PDE mRNA in various bovine tissues was examined by Northern blot hybridization.

Polyadenylated RNA was purified from total RNA preparations using the Poly(A) Quick™ mRNA purification kit (Stratagene) according to the manufacturer's protocol. RNA samples (5 μg) were loaded onto a 1.2% agarose, 6.7% formaldehyde gel. Electrophoresis and RNA transfer were performed as previously described in Sonnenburg et al., supra. Prehybridization of the RNA blot was carried out for 4 hours at 45° C. in a solution containing 50% formamide, 5×SSC, 25 mM sodium phosphate, pH 7, 2× Denhardt's solution, 10% dextran sulfate, and 0.1 mg/ml yeast tRNA. A random hexanucleotide-primer-labelled probe (5×10⁸ cpm/μg) was prepared as described in Feinberg et al., supra, using the 4.7 kb cGB-8 cDNA clone of Example 2 excised by digestion with AccI and SacII. The probe was heat denatured and injected into a blotting bag (6×10⁵ cpm/ml) following prehybridization. The Northern blot was hybridized overnight at 45° C., followed by one 15 minute wash with 2×SSC, 0.1% SDS at room temperature, and three 20 minute washes with 0.1×SSC, 0.1% SDS at 45° C. The blot was exposed to X-ray film for 24 hours at −70° C. The size of the RNA that hybridized with the cGB-PDE probe was estimated using a 0.24-9.5 kb RNA ladder that was stained with ethidium bromide and visualized with UV light.

The ³²P-labelled cGB-PDE cDNA hybridized to a single 6.8 kb bovine lung RNA species. A mRNA band of the identical size was also detected in polyadenylated RNA isolated from bovine trachea, aorta, kidney and spleen.

### Example 4

The following example describes expression of the bovine cGB-PDE cDNA in COS cells (ATCC CRL1651).

A portion of the cGB-8 cDNA was isolated following digestion with the restriction enzyme XbaI. XbaI cut at a position in the pBluescript polylinker sequence located 30 bp upstream of the 5′ end of the cGB-8 insert and at position 3359 within the cGB-8 insert. The resulting 3389 bp fragment, which contains the entire coding region of cGB-8, was then ligated into the unique XbaI cloning site of the expression vector pCDM8 (Invitrogen, San Diego, Calif.). The pCDM8 plasmid is a 4.5 kb eucaryotic expression vector containing a cytomegalovirus promoter and enhancer, an SV40-derived origin of replication, a polyadenylation signal, a procaryotic origin of replication (derived from pBR322) and a procaryotic genetic marker (supF). E. coli MC1061/P3 cells (Invitrogen) were transformed with the resulting ligation products, and transformation positive colonies were screened for proper orientation of the cGB-8 insert using PCR and restriction enzyme analysis. The resulting expression construct containing the cGB-8 insert in the proper orientation is referred to as pCDM8-cGB-PDE.

The pCDM8-cGB-PDE DNA was purified from large-scale plasmid preparations using Qiagen pack-500 columns (Chatsworth, Calif) according to the manufacturer's protocol. COS-7 cells were cultured in Dulbecco's modified Eagle's medium (DMEM) containing 10% fetal bovine serum, 50 μ/ml penicillin and 50 μg/ml streptomycin at 37° C. in a humidified 5% CO₂ atmosphere. Approximately 24 hours prior to transfection, confluent 100 mm dishes of cells were replated at one-fourth or one-fifth the original density. In a typical transfection experiment, cells were washed with buffer containing 137 mM NaCl, 2.7 mM KCl, 1.1 mM potassium phosphate, and 8.1 mM sodium phosphate, pH 7.2 (PBS). Then 4-5 ml of DMEM containing 10% NuSerum (Collaborative Biomedical Products, Bedford, Mass.) was added to each plate. Transfection with 10 μg pCDM8-cGB-PDE DNA or pCDM8 vector DNA mixed with 400 μg DEAE-dextran (Pharmacia) in 60 μl TBS [Tris-buffered saline: 25 mM Tris-HCl (pH 7.4), 137 mM NaCl, 5 mM KCl, 0.6 mM Na₂HPO₄, 0.7 mM CaCl₂, and 0.5 mM MgCl₂] was carried out by dropwise addition of the mixture to each plate. The cells were incubated at 37° C., 5% CO₂ for 4 hours, and then treated with 10% dimethyl sulfoxide in PBS for 1 minute. After 2 minutes, the dimethyl sulfoxide was removed, the cells were washed with PBS and incubated in complete medium. After 48 hours, cells were suspended in 0.5-1 ml of cold homogenization buffer [40 mM Tris-HCl (pH 7.5), 15 mM benzamidine, 15 mM beta-mercaptoethanol, 0.7 μg/ml pepstatin A, 0.5 μg/ml leupeptin, and 5 μM EDTA] per plate of cells, and disrupted using a Dounce homogenizer. The resulting whole-cell extracts were assayed for phosphodiesterase activity, cGMP-binding activity, and total protein concentration as described below in Example 5.

### Example 5

The following example presents results of assays of the cGB-PDE COS cell expression product for phosphodiesterase activity, cGMP-binding activity and Zn²⁺ hydrolase activity.

Phosphodiesterase activity in extracts of the transfected COS cells of Example 4 or in extracts of mock transfected COS cells was measured using a modification of the assay procedure described for the cGs-PDE in Martins et al., J. Biol. Chem., 257: 1973-1979 (1982). Cells were harvested and extracts prepared 48 hours after transfection. Incubation mixtures contained 40 mM MOPS buffer (pH 7), 0.8 mM EDTA, 15 mM magnesium acetate, 2 mg/ml bovine serum albumin, 20 μM [³H]cGMP or [³H]cAMP (100,000-200,000 cpm/assay) and COS-7 cell extract in a total volume of 250 μl. The reaction mixture was incubated for 10 minutes at 30° C., and then stopped by boiling. Next, 10 μl of 10 mg/ml Crotalus atrox venom (Sigma) was added followed by a 10 minute incubation at 30° C. Nucleoside products were separated from unreacted nucleotides as described in Martins et al., supra. In all studies, less than 15% of the total [³H]cyclic nucleotide was hydrolyzed during the reaction.

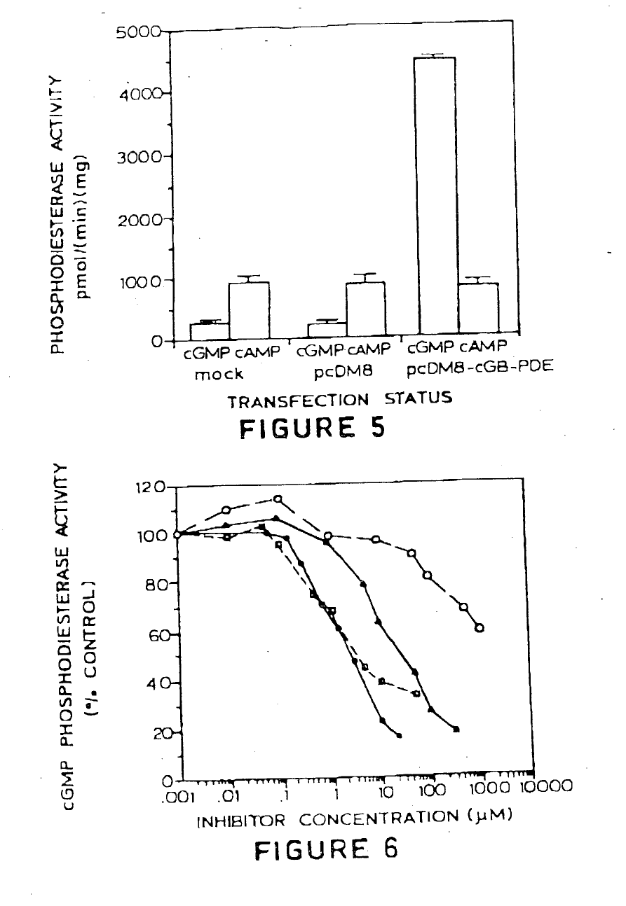

The results of the assays are presented in FIG. 5 wherein the results shown are averages of three separate transfections. Transfection of COS-7 cells with pCDM8-cGB-PDE DNA resulted in the expression of approximately 15-fold higher levels of cGMP phosphodiesterase activity than in mock-transfected cells or in cells transfected with pCDM8 vector alone. No increase in cAMP phosphodiesterase activity over mock or vector-only transfected cells was detected in extracts from cells transfected with pCDM8-cGB-PDE DNA. These results confirm that the cGB-PDE bovine cDNA encodes a cGMP-specific phosphodiesterase.

Extracts from the transfected COS cells of Example 4 were also assayed for cGMP PDE activity in the presence of a series of concentrations of the PDE inhibitors zaprinast, dipyridamole (Sigma), isobutyl-1-methyl-8-methoxymethyLxanthine (MeOxMeMIX) and rolipram.

The results of the assays are presented in FIG. 6 wherein PDE activity in the absence of inhibitor is taken as 100% and each data point represents the average of two separate determinations. The relative potencies of PDE inhibitors for inhibition of cGMP hydrolysis by the expressed cGB-BPDE cDNA protein product were identical to those relative potencies reported for native cGB-PDE purified from bovine lung (Thomas et al., supra). IC₅₀ values calculated from the curves in FIG. 6 are as follows: zaprinast (closed circles), 2 μM; dipyridamole (closed squares), 3.5 μM; MeOxMeMIX (closed triangles), 30 μM; and rolipram (open circles), >300 μM. The IC₅₀ value of zaprinast, a relatively specific inhibitor of cGMP-specific phosphodiesterases, was at least two orders of magnitude lower than that reported for inhibition of phosphodiesterase activity of the cGs-PDE or of the cGMP-inhibited phosphodiesterase (cGi-PDEs) (Reeves et al., pp. 300-316 in Beavo et al., supra). Dipyrimadole, an effective inhibitor of selected cAMP- and cGMP-specific phosphodiesterases, was also a potent inhibitor of the expressed cGB-PDE. The relatively selective inhibitor of calcium/calmodulin-stimulated phosphodiesterase (CaM-PDEs), MeOxMeMIX, was approximately 10-fold less potent than zaprinast and dipyridamole, in agreement with results using cGB-PDE activity purified from bovine lung. Rolipram, a potent inhibitor of low Kₘ cAMP phosphodisterases, was a poor inhibitor of expressed cGB-PDE cDNA protein product. These results show that the cGB-PDE cDNA encodes a phosphodiesterase that possesses catalytic activity characteristic of cGB-PDE isolated from bovine tissue, thus verifying the identity of the cGB-8 cDNA clone as a cGB-PDE.

It is of interest to note that although the relative potencies of the PDE inhibitors for inhibition of cGMP hydrolysis were identical for the recombinant and bovine isolate cGB-PDE, the absolute IC₅₀ values for all inhibitors tested were 2-7 fold higher for the recombinant cGB-PDE. This difference could not be attributed to the effects of any factors present in COS-7 cell extracts on cGMP hydrolytic activity, since cGB-PDE isolated from bovine tissue exhibited identical kinetics of inhibition as a pure enzyme, or when added back to extracts of mock-transfected COS-7 cells. This apparent difference in pharmacological sensitivity may be due to a subtle difference in the structure of the recombinant cGB-PDE cDNA protein product and bovine lung cGB-PDE, such as a difference in post-translational modification at or near the catalytic site. Alternatively, this difference may be due to an alteration of the catalytic activity of bovine lung cGB-PDE over several purification steps.

Cell extracts were assayed for [³H]cGMP-binding activity in the absence or presence of 0.2 mM 3-isobutyl-1-methylaxanthine (IBMX) (Sigma), a competitive inhibitor of cGMP hydrolysis. The cGMP binding assay, modified from the assay described in Thomas I, supra, was conducted in a total volume of 80 μl. Sixty μl of cell extract was combined with 20 μl of a binding cocktail such that the final concentration of components of the mixture were 1 μM[[³H]cGMP, 5 μM cAMP, and 10 μM 8-bromo-cGMP. The cAMP and 8-bromo-cGMP were added to block [³H]cGMP binding to cAK and cGK, respectively. Assays were carried out in the absence and presence of 0.2 mM IBMX. The reaction was initiated by the addition of the cell extract, and was incubated for 60 minutes at 0° C. Filtration of the reaction mixtures was carried out as described in Thomas I, supra. Blanks were determined by parallel incubations with homogenization buffer replacing cell extracts, or with a 100-fold excess of unlabelled cGMP. Similar results were obtained with both methods. Total protein concentration of the cell extracts was determined by the method of Bradford, (Anal. Biochem., 72:248-254 (1976)), using bovine serum albumin as the standard.

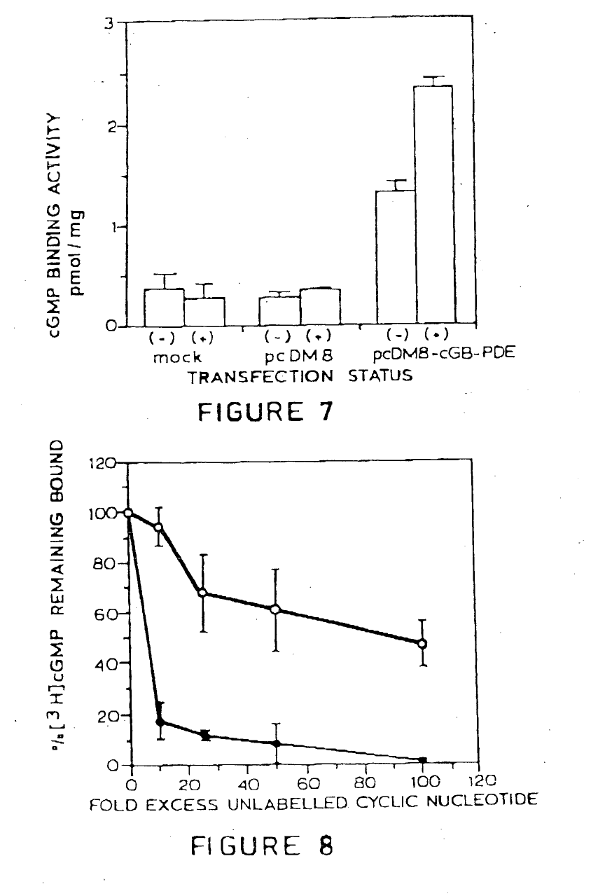

Results of the assay are set out in FIG. 7. When measured at 1 μM [³H]cGMP in the presence of 0.2 mM IBMX, extracts from COS-7 cells transfected with pCDM8-cGB-PDE exhibited 8-fold higher cGMP-binding activity than extracts from mock-transfected cells. No IBMX stimulation of background cGMP binding was observed suggesting that little or no endogenous cGB-PDE was present in the COS-7 cell extracts. In extracts of pCDM8-cGB-PDE transfected cells cGMP-specific activity was stimulated approximately 1.8-fold by the addition of 0.2 mM IBMX. The ability of IBMX to stimulate cGMP binding 2-5 fold is a distinctive property of the cGMP-binding phosphodisterases.

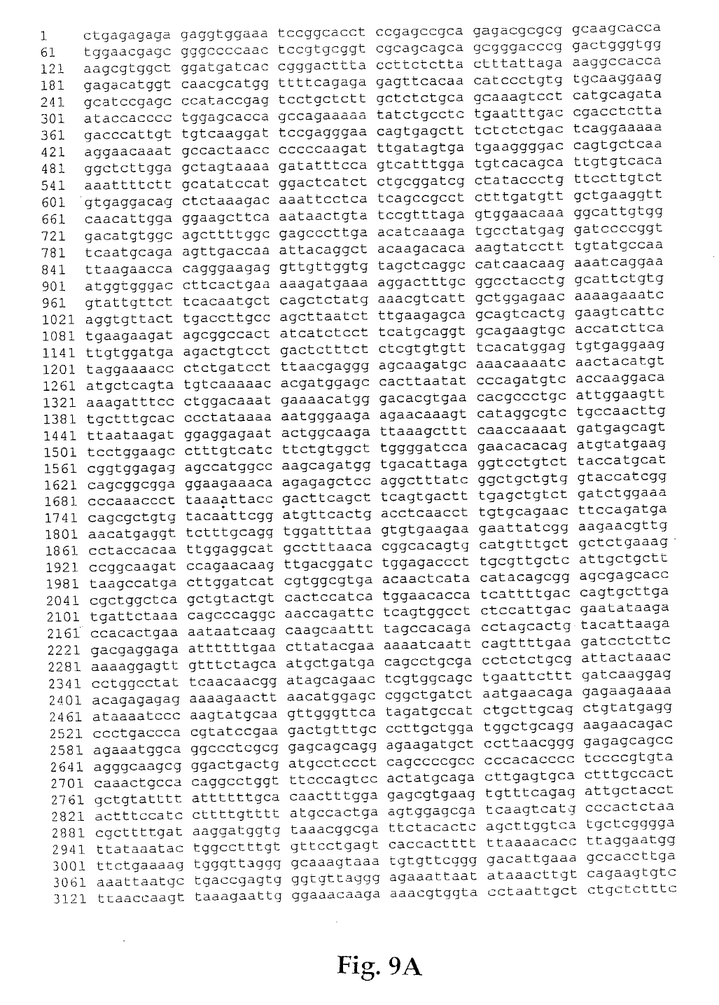

Cell extracts were assayed as described above for [³H]cGMP-binding activity (wherein concentration of [³H]cGMP was 2.5 μM) in the presence of excess unlabelled cAMP or cGMP. Results are presented in FIG. 8 wherein cGMP binding in the absence of unlabelled competitor was taken as 100% and each data point represents the average of three separate determinations. The binding activity of the protein product encoded by the cGB-PDE cDNA was specific for cGMP relative to cAMP. Less than 10-fold higher concentrations of unlabelled cGMP were required to inhibit [³H]cGMP binding activity by 50% whereas approximately 100-fold higher concentrations of cAMP were required for the same degree of inhibition.

The results presented in this example show that the cGB-PDE cDNA encodes a phosphodiesterase which possesses biochemical activities characteristic of native cGB-PDE.

The catalytic domains of mammalian PDEs and a Drosophila PDE contain two tandem conserved sequences (HX₃ HX_{24 26} E) that are typical Zn²⁺-binding motifs in Zn²⁺ hydrolases such as thermolysin (Vallee and Auld, Biochem., 29: 5647-5659 (1990)). cGB-PDE binds Zn²⁺ in the presence of large excesses of Mg²⁺, Mn²⁺, Fe²⁺, Fe³⁺, Ca²⁺ or Cd²⁺. In the absence of added metal, cGB-PDE has a PDE activity that is approximately 20% of the maximum activity that occurs in the presence of 40 mM Mg²⁺, and this basal activity is inhibited by 1,10-phenanthroline or EDTA. This suggests that a trace metal(s) accounts for the basal PDE activity despite exhaustive treatments to remove metals. PDE activity is stimulated by addition of Zn²⁺ (0.02-1 μM) or Co²+(1-20 EM), but not by Fe²⁺, Fe³⁺, Ca²⁺, Cd²⁺, or Cu²⁺. Zn increases the basal PDE activity up to 70% of the maximum stimulation produced by 40 mM Mg²⁺. The stimulatory effect of Zn²⁺ in these assays may be compromised by an inhibitory effect that is caused by Zn²⁺ concentrations>1 μM. The Zn²⁺-supported PDE activity and Zn²⁺ binding by cGB-PDE occur at similar concentrations of Zn²⁺ cGB-PDE thus appears to be a Zn²⁺ hydrolase and Zn²⁺ appears to play a critical role in the activity of the enzyme. See, Colbran et al., The FASEB J., 8: Abstract 2148 (Mar. 15, 1994).

### Example 6

The following example describes the isolation of human cDNAs homologous to the bovine cGB-PDE cDNA.

Several human cDNA clones, homologous to the bovine cDNA clone encoding cGB-PDE, were isolated by hybridization under stringent conditions using a nucleic acid probe corresponding to a portion of the bovine cGB-8 clone (nucleotides 489-1312 of SEQ ID NO: 9).

Isolation of cDNA Fragments Encoding Human cGB-PDE

Three human cDNA libraries (two glioblastoma and one lung) in the vector lambda Zap were probed with the bovine cGB-PDE sequence. The PCR-generated clone corresponding to nucleotides 484-1312 of SEQ ID NO: 9 which is described in Example 1 was digested with EcoRI and SalI and the resulting 0.8 kb cDNA insert was isolated and purified by agarose gel electrophoresis. The fragment was labelled with radioactive nucleotides using a random primed DNA labelling kit (Boehringer).

The cDNA libraries were plated on 150 mm petri plates at a density of approximately 50,000 plaques per plate. Duplicate nitrocellulose filter replicas were prepared. The prehybridization buffer was 3×SSC, 0.1% sarkosyl, 10× Denhardt's, 20 mM sodium phosphate (pH 6.8) and 50 μg/ml salmon testes DNA. Prehybridization was carried out at 65° C. for a minimum of 30 minutes. Hybridization was carried out at 65° C. overnight in buffer of the same composition with the addition of 1-5×10⁵ cpm/ml of probe. The filters were washed at 65° C. in 2×SSC, 0.1% SDS. Hybridizing plaques were detected by autoradiography. The number of cDNAs that hybridized to the bovine probe and the number of cDNAs screened are indicated in Table 2 below.

TABLE 2

| cDNA Library | Type | Positive Plaques | Plaques Screened |
|---|---|---|---|
| Human SW 1088 | dT-primed | 1 | 1.5 × 10⁶ |
| glioblastoma | | | |
| Human lung | dT-primed | 2 | 1.5 × 10⁶ |
| Human SW 1088 | dT-primed | 4 | 1.5 × 10⁶ |
| glioblastoma | | | |

Plasmids designated cgbS2.1, cgbS3.1, cgbL23.1, cgbL27.1 and cgbS27.1 were excised in vivo from the lambda Zap clones and sequenced.

Clone cgbS3.1 contains 2060 bp of a PDE open reading frame followed by a putative intron. Analysis of clone cgbS2.1 reveals that it corresponds to clone cgbS3.1 positions 664 to 2060 and extends the PDE open reading frame an additional 585 bp before reading into a putative intron. The sequences of the putative 5′ untranslated region and the protein encoding portions of the cgbS2.1 and cgbS3.1 clones are set out in SEQ ID NOs: 11 and 12, respectively. Combining the two cDNAs yields a sequence containing approximately 2.7 kb of an open reading encoding a PDE. The three other cDNAs did not extend any further 5′ or 3′ than cDNA cgbS3.1 or cDNA cgbS2.1.

To isolate additional cDNAs, probes specific for the 5′ end of clone cgbS3.1 and the 3′ end of clone cgbS2.1 were prepared and used to screen a SW1088 glioblastoma cDNA library and a human aorta cDNA library. A 5′ probe was derived from clone cgbS3.1 by PCR using the primers cgbS3.1S311 and cgbL23.1A1286 whose sequences are set out in SEQ ID NOs: 8 and 9, respectively, and below.

| | |
|---|---|
| Primer cgbS3.1S311 | |
| 5′ GCCACCAGAGAAATGGTC 3′ | (SEQ ID NO: 13) |
| Primer cgbL23.11A1286 | |
| 5′ ACAATGGGTCTAAGAGGC 3′ | (SEQ ID NO: 14) |

The PCR reaction was carried out in a 50 μl reaction volume containing 50 pg cgbS3.1 cDNA, 0.2 mM dNTP, 10 μg/ml each primer, 50 mM KCl, 10 mM Tris-HCl pH 8.2, 1.5 MM MgCl₂ and Taq polymerase. After an initial four minute denaturation at 94° C., 30 cycles of one minute at 94° C., two minutes at 50° C. and four minutes at 72° C. were carried out. An approximately 0.2 kb fragment was generated by the PCR reaction which corresponded to nucleotides 300-496 of clone cgbS3.1.

A 3′ probe was derived from cDNA cgbS2.1 by PCR using the oligos cgbL23.1S1190 and cgbS2.1A231 whose sequences are set out below.

| | |
|---|---|
| Primer cgbL23.1S1190 | |
| 5′ TCAGTGCATGTTTGCTGC 3′ | (SEQ ID NO: 15) |
| Primer cgbS2.1A231 | |
| 5′ TACAAACATGTTCATCAG 3′ | (SEQ ID NO: 16) |

The PCR reaction as carried out similarly to that described above for generating the 5′ probe, and yielded a fragment of approximately 0.8 kb corresponding to nucleotides 1358-2139 of cDNA cgbS2.1. The 3′ 157 nucleotides of the PCR fragment (not shown in SEQ ID NO: 12) are within the presumptive intron.

The two PCR fragments were purified and isolated by agarose gel electrophoresis, and were labelled with radioactive nucleotides by random priming. A random-primed SW1088 glioblastoma cDNA library (1.5×10⁶ plaques) was screened with the labelled fragments as described above, and 19 hybridizing plaques were isolated. An additional 50 hybridizing plaques were isolated from a human aorta cDNA library (dT and random primed, Clontech, Palo Alto, Calif.).

Plasmids were excised in vivo from some of the positive lambda Zap clones and sequenced. A clone designated cgbS53.2, the sequence of which is set out in SEQ ID NO: 17, contains an approximately 1.1 kb insert whose sequence overlaps the last 61 bp of cgbS3.1 and extends the open reading frame an additional 135 bp beyond that found in cgbS2.1. The clone contains a termination codon and approximately 0.3 kB of putative 3′ untranslated sequence.

Generation of a Composite cDNA Encoding Human cGB-PDE

Clones cgbS3.1, cgbS2.1 and cgbS53.2 were used as described in the following paragraphs to build a composite cDNA that contained a complete human cGB-PDE opening reading frame. The composite cDNA is designated cgbmetls156-2 and was inserted in the yeast ADH1 expression vector pBNY6N.

First, a plasmid designated cgb stop-2 was generated that contained the 3′ end of the cGB-PDE open reading frame. A portion of the insert of the plasmid was generated by PCR using clone cgbS53.2 as a template. The PCR primers utilized were cgbS2.1S1700 and cgbstop-2.

| | |
|---|---|
| Primer cgbS2.1S1700 | |
| 5′ TTTGGAAGATCCTCATCA 3′ | (SEQ ID NO: 18) |
| Primer cgbstop-2 | |
| 5′ ATGTCTCGAGTCAGTTCCGCTTGGCCTG 3′ | (SEQ ID NO: 19) |

The PCR reaction was carried out in 50 μl containing 50 pg template DNA, 0.2 mM dNTPs, 20 mM Tris-HCl pH 8.2, 10 mM KCl, 6 mM (NH₄)₂SO₄, 1.5 mM MgCl₂, 0.1% Triton-X-100, 500 ng each primer and 0.5 units of Pfu polymerase (Stratagene). The reaction was heated to 94° C. for 4 minutes and then 30 cycles of 1 minute at 94° C., 2 minutes at 50° C. and four minutes at 72° C. were performed. The polymerase was added during the first cycle at 50° C. The resulting PCR product was phenol/chloroform extracted, chloroform extracted, ethanol precipitated and cut with the restriction enzymes BclI and XhoI. The restriction fragment was purified on an agarose gel and eluted.

This fragment was ligated to the cDNA cgbS2.1 that had been grown in dam E. coli, cut with the restriction enzymes BclI and XhoI, and gel-purified using the Promega magic PCR kit. The resulting plasmid was sequenced to verify that cgbstop-2 contains the 3′ portion of the cGB-PDE open reading frame,

Second, a plasmid carrying the 5′ end of the human cGB-PDE open reading frame was generated. Its insert was generated by PCR using clone cgbS3.1 as a template. PCR was performed as described above using primers cgbmet156 and cgbS2.1A2150.

Primer cgbmet1156

(SEQ ID NO: 20)

5′ TACAGAATTCTGACCATGGAGCGGGCCGGC 3′
Primer cgbS2.1A2150

(SEQ ID NO: 21)

5′ CATTCTAAGCGGATACAG 3′

The resulting PCR fragment was phenol/choloform extracted, choloform extracted, ethanol precipitated and purified on a Sepharose CL-6B column. The fragment was cut with the restriction enzymes EcoRV and EcoRI, run on an agarose gel and purified by spinning through glass wool. Following phenol/chloroform extraction, chloroform extraction and ethanol precipitation, the fragment was ligated into EcoRI/EcoRV digested BluescriptII SK(+) to generate plasmid cgbmet156. The DNA sequence of the insert and junctions was determined. The insert contains a new EcoRI site and an additional 5 nucleotides that together replace the original 155 nucleotides 5′ of the initiation codon. The insert extends to an EcoRV site beginning 531 nucleotides from the initiation codon.

The 5′ and 3′ portions of the cGB-PDE open reading frame were then assembled in vector pBNY6a. The vector pBNY6a was cut with EcoRI and XhoI, isolated from a gel and combined with the agarose gel purified EcoRI/EcoRV fragment from cgbmet156 and the agarose gel purified EcoRV/XhoI fragment from cgbstop-2. The junctions of the insert were sequenced and the construct was named hcbgmet156-2 6a.

The cGB-PDE insert from hcbgmet156-2 6a was then moved into the expression vector pBNY6n. Expression of DNA inserted in this vector is directed from the yeast ADH1 promoter and terminator. The vector contains the yeast 2 micron origin of replication, the pUC19 origin of replication and an ampicillin resistance gene. Vector pBNY6n was cut with EcoRI and XhoI and gel-purified. The EcoRI/XhoI insert from hcgbmet156-2 6a was gel purified using Promega magic PCR columns and ligated into the cut pBNY6n.

All new junctions in the resulting construct, hcgbmet156-2 6n, were sequenced. The DNA and deduced amino acid sequences of the insert of hcgbmet15-2 6n which encodes a composite human cGB-PDE is set out in SEQ ID NOs: 22 and 23. The insert extends from the first methionine in clone cgbS3.1 (nucleotide 156) to the stop codon (nucleotide 2781) in the composite cDNA. Because the methionine is the most 5′ methionine in clone cgbS3.1 and because there are no stop codons in frame with the methionine and upstream of it, the insert in pBNY6n may represent a truncated form of the open reading frame.

Variant cDNAs

Four human cGB-PDE cDNAs that are different from the hcgbmet156-2 6n composite cDNA have been isolated. One cDNA, cgbL23.1, is missing an internal region of hcgbmet156-2 6n (nucleotides 997-1000 to 1444-1447). The exact end points of the deletion cannot be determined from the cDNA sequence at those positions. Three of the four variant cDNAs have 5′ end sequences that diverge from the hcgbmet156-2 6n sequence upstream of nucleotide 151 (cDNAs cgbA7f, cgbA5C, cgb12). These cDNAs presumably represent alternatively spliced or unspliced mRNAs.

### Example 7

The following example describes expression of human cGB-PDE cDNA in yeast cells.

The composite human cGB-PDE cDNA construct, hcgbmet156-2 6n, was transformed into the yeast strain YKS45 (ATCC 74225) MATU. hisα trp1 ura3 leu3 pde1::HIS3 pde2::TRP1) in which two endogenous PDE genes are deleted. Transformants complementing the leu deficiency of the YKS45 strain were selected and assayed for cGB-PDE activity. Extracts from cells bearing the plasmid hcgbmet156-2 6n were determined to display cyclic GMP-specific phosphodiesterase activity by the assay described below.

One liter of YKS45 cells transformed with the plasmid cgbmet156-2 6n and grown in SC-leu medium to a density of 1-2×10⁷ cells/ml was harvested by centrifugation, washed once with deionized water, frozen in dry ice/ethanol and stored at −70° C. Cell pellets (1-1.5 ml) were thawed on ice in the presence of an equal volume of 25 mM Tris-Cl (pH 8.0)/5 mM EDTA/5 mM EGTA/1 mM o-phenanthroline/0.5 mM AEBSF (Calbiochem)/0.1% β-mercaptoethanol and 10 μg/ml each of aprotinin, leupeptin, and pepstatin A. The thawed cells were added to 2 ml of acid-washed glass beads (425-600 μM, Sigma) in 15 ml Corex tube. Cells were broken with 4 cycles consisting of a 30 second vortexing on setting 1 followed by a 60 second incubation on ice. The cell lysate was centrifuged at 12,000× g for 10 minutes and the supernatant was passed through a 0.8 μfilter. The supernatant was assayed for cGMP PDE activity as follows. Samples were incubated for 20 minutes at 30° C. in the presence of 45 mM Tris-Cl (pH 8.0), 2 mM EGTA, 1 mM EDTA, 0.2 mg/ml BSA, 5 mM MgCl₂, 0.2 mM o-phenanthroline, 2 μg/ml each of pepstatin A, leupeptin, and aprotinin, 0.1 mM AEBSF, 0.02% β-mercaptoethanol and 0.1 mM [³H]cGMP as substrate. [¹⁴C]-AMP (0.5 nCi/assay) was added as a recovery standard. The reaction was terminated with stop buffer (0.1M ethanolamine pH 9.0, 0.5M ammonium sulfate, 10 mM EDTA, 0.05% SDS final concentration). The product was separated from the cyclic nucleotide substrate by chromatography on BioRad Affi-Gel 601. The sample was applied to a column containing approximately 0.25 ml of Affi-Gel 601 equilibrated in column buffer (0.1M ethanolamine pH 9.0 containing 0.5M ammonium sulfate). The column was washed five times with 0.5 ml of column buffer. The product was eluted with four 0.5 ml aliquots of 0.25 M acetic acid and mixed with 5 ml Ecolume (ICN Biochemicals). The radioactive product was measured by scintillation counting.

### Example 8

The following example demonstrates expression of cGB-PDE mRNA in human tissues by RNase protection assay.

A probe corresponding to a portion of the putative cGMP binding domain of cGB-PDE (402 bp corresponding to nucleotides 1450 through 1851 of SEQ ID NO: 13) was generated by PCR. The PCR fragment was inserted into the EcoRI site of the plasmid pBSII SK(−) to generate the plasmid RP3. RP3 plasmid DNA was linearized with XbaI and antisense probes were generated by a modification of the Stratagene T7 RNA polymerase kit. Twenty-five ng of linearized plasmid was combined with 20 microcuries of alpha ³²P rUTP (800 Ci/mmol, 10 mCi/ml), IX transcription buffer (40 mM TrisCl, pH 8, 8 mM MgCl₂, 2 mM spermidine, 50 mM NaCl), 0.25 mM each rATP, rGTP and rCTP, 0.1 units of RNase Block II, 5 mM DTT, 8 μM rUTP and 5 units of T7 RNA Polymerase in a total volume of 5 μl. The reaction was allowed to proceed 1 hour at room temperature and then the DNA template was removed by digestion with RNase free DNase. The reaction was diluted into 100 μl of 40 mM TrisCl, pH 8, 6 mM MgCl₂ and 10 mM NaCl. Five units of RNase-free DNase were added and the reaction was allowed to continue another 15 minutes at 37° C. The reaction was stopped by a phenol extraction followed by a phenol chloroform extraction. One half volume of 7.5M NH₄OAc was added and the probe was ethanol precipitated.

The RNase protection assays were carried out using the Ambion RNase Protection kit (Austin, Tex.) and 10 μg RNA isolated from human tissues by an acid guanidinium extraction method. Expression of cGB-PDE mRNA was easily detected in RNA extracted from skeletal muscle, uterus, bronchus, skin, right saphenous vein, aorta and SW1088 glioblastoma cells. Barely detectable expression was found in RNA extracted from right atrium, right ventricle, kidney cortex, and kidney medulla. Only complete protection of the RP3 probe was seen. The lack of partial protection argues against the cDNA cgbL23.1 (a variant cDNA described in Example 7) representing a major transcript, at least in these RNA samples.

### Example 9

The following example describes the bacterial expression of human cGB-PDE cDNA and the development of antibodies reactive with the bacterial cGB-PDE expression product.

Polyclonal antisera was raised to E. coli-produced fragments of the human cGB-PDE.

A portion of the human cGB-PDE cDNA (nucleotides 1668-2612 of SEQ ID NO: 22, amino acids 515-819 of SEQ ID NO: 23) was amplified by PCR and inserted into the E. coli expression vector pGEX2T (Pharmacia) as a BamHI/EcoRI fragment. The pGEX2T plasmid carries an ampicillin resistance gene, an E. coli laq I^{q} gene and a portion of the Schistosoma japonicum glutathione-S-transferase (GST) gene. DNA inserted in the plasmid can be expressed as a fusion protein with GST and can then be cleaved from the GST portion of the protein with thrombin. The resulting plasmid, designated cgbPE3, was transformed into E. coli strain LE392 (Stratagene). Transformed cells were grown at 37° C. to an OD600 of 0.6. IPTG (isopropylthiogalactopyranoside) was added to 0.1 mM and the cells were grown at 37° C. for an additional 2 hours. The cells were collected by centrifugation and lysed by sonication. Cell debris was removed by centrifugation and the supernatant was fractionated by SDS-PAGE. The gel was stained with cold 0.4M KCl and the GST-cgb fusion protein band was excised and electroeluted. The PDE portion of the protein was separated from the GST portion by digestion with thrombin. The digest was fractionated by SDS-PAGE, the PDE protein was electroeluted and injected subcutaneously into a rabbit. The resultant antisera recognizes both the bovine cGB-PDE fragment that was utilized as antigen and the full length human cGB-PDE protein expressed in yeast (see Example 8).

### Example 10

The following example describes ploynucleotides encoding cGB-PDE analogs and their fragments. The polynucleotides encoding various cGB-PDE analogs and cGB-PDE fragments were generated by standard methods.

A. cGB-PDE Analogs

All known cGMP-binding PDEs contain two internally homologous tandem repeats within their putative cGMP-binding domains. In the bovine cGB-PDE of the invention, the repeats span at least residues 228-311 (repeat A) and 410-500 (repeat B) of SEQ ID NO: 10. Site-directed mutagenesis of an aspartic acid that is conserved in repeats A and B of all known cGMP-binding PDEs was used to create analogs of cGB-PDE having either Asp-289 replaced with Ala (D289A) or Asp-478 replaced with Ala (D478A). Recombinant wild type (WT) bovine and mutant bovine cGB-PDEs were expressed in COS-7 cells. cGB-PDE purified from bovine lung (native cGB-PDE) and WT cGB-PDE displayed identical cGMP-binding kinetics with a K_{d} of approximately 2 μM and a curvilinear dissociation profile (t_{1/2}=1.3 hours at 4° C.). This curvilinearity may have been due to the presence of distinct high affinity (slow) and low affinity (fast) sites of cGMP binding. The D289A mutant had significantly decreased affinity for cGMP (K_{d}>20 μM) and a single rate of cGMP-association (t_{1/2}=0.5 hours), that was similar to that calculated for the fast site of WT and native cGB-PDE. This suggested the loss of a slow cGMP-binding site in repeat A of this mutant. Conversely, the D478A mutant showed higher affinity for cGMP (K_{d} of approximately 0.5 μM) and a single cGMP-dissociation rate (t_{1/2}=2.8 hours) that was similar to the calculated rate of the slow site of WT and native cGB-PDE. This suggested the loss of a fast site when repeat B was modified. These results indicate that dimeric cGB-PDE possesses two homologous but kinetically distinct cGMP-binding sites, with the conserved aspartic acid being critical for interaction with cGMP at each site. See, Colbran et al., FASEB J., 8: Abstract 2149 (May 15, 1994).

B. Amino-Terminal Truncated cGB-PDE Polypeptides

A truncated human cGB-PDE polypeptide including amino acids 516-875 of SEQ ID NO: 23 was expressed in yeast. A cDNA insert extending from the NcoI site at nucleotide 1555 of SEQ ID NO: 22 through the XhoI site at the 3′ end of SEQ ID NO: 22 was inserted into the ADH2 yeast expression vector YEpC-PADH2d (Price et al., Meth. Enzymol., 185: 308-318 (1990)) that had been digested with NcoI and SalI to generate plasmid YEpC-PADH2d HcGB. The plasmid was transformed into spheroplasts of the yeast strain yBJ2-54 (prc1-407 prb1-1122 pep4-3 leu2 trp1 ura3-52 Δpde1:URA3, HIS3 Δpde2::TRP1 cir.). The endogenous PDE genes are deleted in this strain. Cells were grown in SC-leu media with 2% glucose to 10⁷ cells/ml, collected by filtration and grown 24 hours in YEP media containing 3% glycerol. Cells were pelleted by centrifugation and stored frozen. Cells were disrupted with glass beads and the cell homogenate was assayed for phosphodiesterase activity essentially as described in Prpic et al., Anal. Biochem., 208: 155-160 (1993). The truncated human cGB-PDE polypeptide exhibited phosphodiesterase activity.

C. Carboxy-Terminal Truncated cGB-PDE Polypeptides

Two different plasmids encoding carboxy-terminal truncated human cGB-PDE polypeptides were constructed.

Plasmid pBJ6-84Hin contains a cDNA encoding amino acids 1-494 of SEQ ID NO: 23 inserted into the NcoI and SalI sites of vector YEpC-PADH2d. The cDNA insert extends from the NcoI site at nucleotide position 10 of SEQ ID NO: 22 through the HindIII site at nucleotide position 1494 of SEQ ID NO: 22 followed by a linker and the SalI site of YEpC-PADH2d.

Plasmid pBJ6-84Ban contains a cDNA encoding amino acids 1-549 of SEQ ID NO: 23 inserted into the NcoI and SalI sites of vector YEpC-PADH2d. The cDNA insert extends from the NcoI site at nucleotide position 10 of SEQ ID NO: 22 through the BanI site at nucleotide position 1657 bf SEQ ID NO: 22 followed by a linker and the SalI site of YEpC-PADH2d.

The truncated cGB-PDE polypeptides are useful for screening for modulators of cGB-PDE activity.

### Example 11

The following example describes the generation of monoclonal antibodies that recognize cGB-PDE.

Yeast yBJ2-54 containing the plasmid YEpADH2HcGB (Example 10B) were fermented in a New Brunswick Scientific 10 liter Microferm. The cGB-PDE cDNA insert in plasmid YEpADH2 HcGB extends from the NcoI site at nucleotide 12 of SEQ ID NO: 22 to the XhoI site at the 3′ end of SEQ ID NO: 22. An inoculum of 4×10⁹ cells was added to 8 liters of media containing SC-leu, 5% glucose, trace metals, and trace vitamins. Fermentation was maintained at 26° C., agitated at 600 rpm with the standard microbial impeller, and aerated with compressed air at 10 volumes per minute. When glucose decreased to 0.3% at 24 hours post-inoculation the culture was infused with 2 liters of 5× YEP media containing 15% glycerol. At 66 hours post-inoculation the yeast from the ferment was harvested by centrifugation at 4,000× g for 30 minutes at 4° C. Total yield of biomass from this fermentation approached 350 g wet weight.

Human cGB-PDE enzyme was purified from the yeast cell pellet. Assays for PDE activity using 1 mM cGMP as substrate was employed to follow the chromatography of the enzyme. All chromatographic manipulations were performed at 4° C.

Yeast (29 g. wet weight) were resuspended in 70 ml of buffer A (25 mM Tris pH 8.0, 0.25 mM DTT, 5 mM MgCl₂, 10 μM ZnSO₄, 1 mM benzamidine) and lysed by passing through a microfluidizer at 22-24,000 psi. The lysate was centrifuged at 10,000× g for 30 minutes and the supernatant was applied to a 2.6×28 cm column containing Pharmacia Fast Flow Q anion exchange resin equilibrated with buffer B containing 20 mM BisTris-propane pH 6.8, 0.25 mM DTT, 1 mM MgCl₂, and 10 μM ZnSO₄. The column was washed with 5 column volumes of buffer B containing 0.125M NaCl and then developed with a linear gradient from 0.125 to 1.0 M NaCl. Fractions containing the enzyme were pooled and applied directly to a 5×20 cm column of ceramic hydroxyapatite (BioRad) equilibrated in buffer C containing 20 mM BisTris-propane pH 6.8, 0.25 mM DTT, 0.25MKCl, 1 mM MgCl₂, and 10 μM ZnSO₄. The column was washed with 5 column volumes of buffer C and eluted with a linear gradient from 0 to 250 mM potassium phosphate in buffer C. The pooled enzyme was concentrated 8-fold by ultrafiltration (YM30 membrane, Amicon). The concentrated enzyme was chromatographed on a 2.6×90 cm column of Pharmacia Sephacryl S300 (Piscataway, N.J.) equilibrated in 25 mM BisTris-propane pH 6.8, 0.25 mM DTT, 0.25M NaCl, 1 mM MgCl₂, and 20 μM ZnSO₄. Approximately 4 mg of protein was obtained. The recombinant human cGB-PDE enzyme accounted for approximately 90% of protein obtained as judged by SDS polyacrylamide gel electrophoresis followed by Coomassie blue staining.

The purified protein was used as an antigen to raise monoclonal antibodies. Each of 19 week old Balb/c mice (Charles River Biotechnical Services, Inc., Wilmington, Mass.) was immunized sub-cutaneously with 50 μg purified human cGB-PDE enzyme in a 200 μl emulsion consisting of 50% Freund's complete adjuvant (Sigma Chemical Co.). Subsequent boosts on day 20 and day 43 were administered in incomplete Freund's adjuvant. A pre-fusion boost was done on day 86 using 50 μg enzyme in PBS. The fusion was performed on day 90.

The spleen from mouse #1817 was removed sterilely and placed in 10 ml serum free RPMI 1640. A single-cell suspension was formed and filtered through sterile 70-mesh Nitex cell strainer (Becton Dickinson, Parsippany, N.J.), and washed twice by centrifuging at 200 g for 5 minutes and resuspending the pellet in 20 ml serum free RPMI. Thymocytes taken from 3 naive Balb/c mice were prepared in a similar manner.

NS-1 myeloma cells, kept in log phase in RPMI with 11% Fetalclone (FBS) (Hyclone Laboratories, Inc., Logan, Utah) for three days prior to fusion, were centrifuged at 200 g for 5 minutes, and the pellet was washed twice as described in the foregoing paragraph. After washing, each cell suspension was brought to a final volume of 10 ml in serum free RPMI, and 20 μl was diluted 1:50 in 1 ml serum free RPMI. 20 μl of each dilution was removed, mixed with 20 μl 0.4% trypan blue stain in 0.85% saline (Gibco), loaded onto a hemocytometer (Baxter Healthcare Corp., Deerfield, Ill.) and counted.

Two×10⁸ spleen cells were combined with 4.0×10⁷ NS-1 cells, centrifuged and the supernatant was aspirated. The cell pellet was dislodged by tapping the tube and 2 ml of 37° C. PEG 1500 (50% in 75 mM Hepes, pH 8.0) (Boehringer Mannheim) was added with stirring over the course of 1 minute, followed by adding 14 ml of serum free RPMI over 7 minutes. An additional 16 ml RPMI was added and the cells were centrifuged at 200 g for 10 minutes. After discarding the supernatant, the pellet was resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×10⁶ thyniocytes/ml. The suspension was first placed in a T225 flask (Coming, United Kingdom) at 37° C. for two hours before being dispensed into ten 96-well flat bottom tissue culture plates (Coming, United Kingdom) at 200 μl/well. Cells in plates were fed on days 3, 4, 5 post fusion day by aspirating approximately 100 μl from each well with an 20 G needle (Becton Dickinson), and adding 100 μl/well plating medium described above except containing 10 units/ml IL-6 and lacking thymocytes.

The fusion was screened initially by ELISA. Immulon 4 plates (Dynatech) were coated at 4° C. overnight with purified recombinant human cGB-PDE enzyme (100 ng/well in 50 mM carbonate buffer pH9.6). The plates were washed 3× with PBS containing 0.05% Tween 20 (PBSI). The supernatants from the individual hybridoma wells were added to the enzyme coated wells (50 μl/well). After incubation at 37° C. for 30 minutes, and washing as above, 50 μl of horseradish peroxidase conjugated goat anti-mouse IgG(fc) (Jackson ImmunoResearch, West Grove, Pa.) diluted 1:3500 in PBST was added. Plates were incubated as above, washed 4× with PBST and 100 μl substrate consisting of 1 mg/ml o-phenylene diamine (Sigma) and 0.1 μl/ml 30% H₂O₂ in 100 mM citrate, pH 4.5, was added. The color reaction was stopped in 5 minutes with the addition of 50 μl of 15% H₂SO₄. A₄₉₀ was read on a plate reader (Dynatech).

Wells C5G, E4D, F1G, F9H, F11G, J4A, and J5D were picked and renamed 102A, 102B, 102C, 102D, 102E, 102F, and 102G respectively, cloned two or three times, successively, by doubling dilution in RPMI, 15% FBS, 100 μM sodium hypoxanathine, 16 μM thymidine, and 10 units/ml IL-6. Wells of clone plates were scored visually after 4 days and the number of colonies in the least dense wells were recorded. Selected wells of the each cloning were tested by ELISA.

The monoclonal antibodies produced by above hybridomas were isotyped in an ELISA assay. Results showed that monoclonal antibodies 102A to 102E were IgG1, 102F was IgG2b and 102G was IgG2a.

All seven monoclonal antibodies reacted with human cGS-PDE as determined by Western analysis.

### Example 12

The following example demonstrates that PDE5 is converted to an activated state upon cGMP binding to the GAF A domain.

Materails and Methods

Cell Culture

Human embryonic kidney (HEK) 293 cells, obtained from the American Type Tissue Culture Collection, were grown in DMEM supplemented with 10% fetal bovine serum, 100 units/ml penicillin, and 100 mg/ml streptomycin at 37° C. in a humidified 5% CO2 atmosphere.

Transient Transfection

Mouse PDE5A1 (GenBank™ accession number NM_{—}153422) was subcloned into pcDNA3 vector (Invitrogen). The plasmids for transfection were purified using a Qiagen Plasmid Maxi Kit (Qiagen®). Cells plated on 100-mm plates were transiently transfected with DNA using the Lipofectamine 2000 (Invitrogen) method according to the manufacturer's protocol. Protein expression was verified by Western blot analysis of cell lysates with total PDE5 antibodies. At 2 days after transfection, cells were harvested for experiments.

After washing three times with cold PBS, cells were lysed in homogenization buffer (50 mM Tris-HCl, pH 7.5, 2.0 mM EDTA, 1 mM DTT, 10 μg/ml aprotinin, 5 μg/ml pepstatin, 20 μg/ml leupeptin, 1 mM benzamidine, 0.2 mM sodium vanadate, 50 mM sodium β-glycerophosphate). Lysates were briefly sonicated (2-3 sec) using a Virsonic 100 sonicator (Virtis Company, NY) and centrifuged at 16,000×g for 15 min. The supernatants were used for activity assays. In some experiments the cell extract was centrifuiged at 100,000×g for 1 h and loaded on a Mono Q anion exchange column HR 5/5 (Pharmacia). HPLC was conducted using conditions published previously (Rybalkin et al., 1997 J Clin Invest, 100, 2611-2621). Fractions containing PDE5 activity were used for experiments.

Site-Directed Mutagenesis of PDE5 Phospho-Site

Site-directed mutagenesis was carried out using the QuickChange™ site-directed mutagenesis kit (Stratagene) according to the manufacturer's protocol. The following oligonucleotide primers were used to substitute serine to alanine:

(SEQ ID NO.: 26)

5′-GCC AGA AAA ATA GCT GCC TCT GAA TTT G-3′
and

(SEQ ID NO.: 27)

5′-C AAA TTC AGA GGC AGC TAT TTT TCT GGC-3′

The mutated nucleotides are underlined. The mutation was confirmed by DNA sequencing.

In vitro Phosphorvlation and Immunoprecipitation

Cell extracts were incubated for 60 min at 30° C. in 30 μl of phosphorylation buffer containing homogenization buffer with 0.1 mM ATP, 1 μCi of ³²P-ATP (specific activity 30 Ci/mmol, Perkin-Elmer Life Sciences, Inc.), 10 mM MgCl₂, 300 units of PKG (“Promega”) or 50 units of catalytic subunit of PKA (“Promega”) and 50 μM cGMP. After phosphorylation samples were diluted in immunoprecipitation buffer: homogenization buffer with 150 mM NaCl, 1% NP-40, and incubated with PDE5 monoclonal antibody, followed by addition of Protein G-agarose beads. After washing three times in immunoprecipitation buffer, the immunoprecipitates were prepared in SDS sample buffer, resolved by SDS-PAGE and analyzed by Western blotting. Western blots were also subjected to autoradiography to detect radiolabeled phosphoproteins. In experiments without radiolabeled ATP, samples were prepared in SDS sample buffer immediately after phosphorylation.

Antibody Characterization

The production and purification of phospho-specific PDE5 rabbit polyclonal antibody, raised against a phosphopeptide (amino acids 85-98 of bovine PDE5A), total PDE5 rabbit polyclonal antibody, raised against a peptide (amino acids 836-852, bovine PDE5A) and mouse monoclonal antibodies, generated using recombinant protein (amino acids 125-539 of bovine PDE5A) were published previously (Rybalkin et al., 2002 J Biol Chem, 277, 3310-3317), incorporated by reference herein.

Monoclonal antibodies used as cell culture supernatants prepared from the hybridomas grown in culture, were additionally purified from culture media by using the Ultrafree-4 centrifugal filter unit (Millipore Corp.).

Western Blot Analysis and Autoradiography

Samples were prepared in 2×SDS-sample buffer, heated at 95° C. for 5 minutes and subjected to 8% SDS-PAGE. The separated proteins were transferred onto nitrocellulose membranes and immunostained with PDE5 antibodies. Immunoreactivity was detected by ECL using horseradish peroxidase conjugated goat anti-rabbit IgGs or goat anti-mouse IgGs and SuperSignal® West Pico Chemiluminescent Substrate. In experiments with radiolabeled ATP the blots were also subjected to autoradiography using Kodak XAR-5 films with intensifying screens for 24-48 hrs at -80° C.

cGMP Saturation Binding Assay

Binding studies were conducted by the Millipore filter binding assay in a total volume of 40 μl containing binding buffer (50 mM Tris-HCl, pH 7.5, 2.0 mM EDTA, 1 mM DTT, 100 mM NaCl) and 0.1 mM IBMX in the presence of 0.01 -20 μM ³H-cGMP. In some experiments an additional 30 min preincubation of recombinant PDE5 with monoclonal antibodies (mAb/P3B2 or mAb/P4D8) was performed before addition of cGMP. Following a 30 min incubation on ice, 1 ml of ice cold binding buffer was added to the assays. The samples were immediately filtered through a Millipore HAWP nitrocellulose membrane (pore size 0.45 μM, 24 mm diameter) and rinsed three times with ice cold binding buffer (10 ml each). The filters were dissolved in 5 ml of Filter Count™ (Packard Instrument) and counted in a liquid scintillation counter.

GAF Domain Protein Analyses

Recombinant proteins of GAF(A+B) (amino acids 125-539 of mouse PDE5), GAF A (amino acids 125-320) and GAF B (amino acids 334-525) were expressed in E. coli with polyhistidine tags and purified on a Talon Metal Affinity Resin (Clontech). Samples (0.1 μg) were resolved using 12% SDS-PAGE gel. Protein bands were either analyzed by Western blotting or silver stained (SilverXpress, Invitrogen) according to the manufacturer's protocol.

PDE Assays and Protein Determinations

Phosphodiesterase assays were carried out according to established procedures. All assays were performed for 5 min (unless otherwise indicated) at 30° C. using different concentrations of cGMP (0.1-10 μM) as substrates. Protein concentrations were determined by the Coomassie Plus protein assay (Pierce).

Results

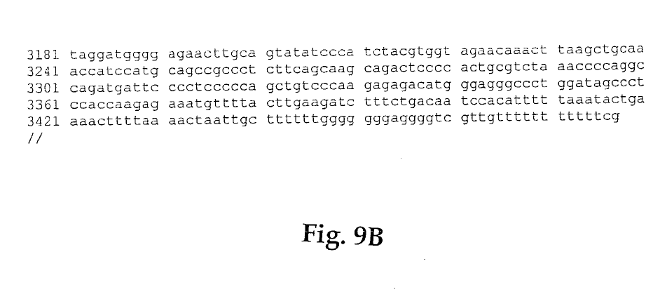

The mouse PDE5 cDNA clone was isolated by screening a mouse lung cDNA library using standard high stringency procedures. The probe used for the screening was made from a full length bovine cGB-PDE cDNA template in pBlueScript. The nucleotide and predicted amino acid sequences of mouse PDE5 are shown in FIGS. 9 (SEQ ID NO. 24) and 10 (SEQ ID NO.: 25), respectively.

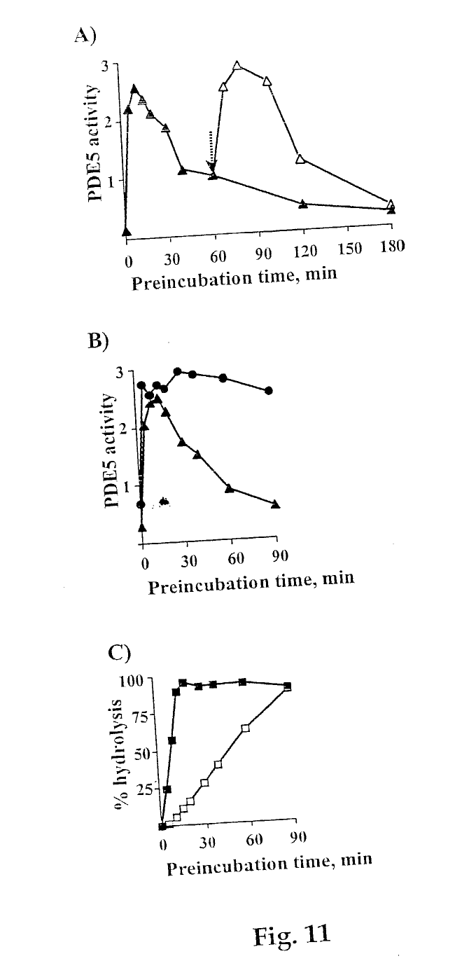

Preincubation of recombinant PDE5 with cGMP results in strong reversible activation of PDE5 catalytic activity at low substrate concentration. Recombinant mouse PDE5 was expressed in HEK 293 cells. The expression level of PDE5 in these cells was 200 times higher than non-transfected cells in all experiments. The cells were lysed in a small volume of homogenization buffer in order to obtain a concentrated extract, and then the cell extract was preincubated with 50 μM cGMP on ice without PDE inhibitors (filled triangles, FIGS. 11A and 11B) or with 0.2 μM sildenafil (filled circles, FIG. 11B). At different time points aliquots were diluted 1/400-1/500, and different amounts of diluted extract (additional dilution 1/5-1/10) were used for PDE5 activity assayed at 0.1 μM cGMP for 5 min at 30° C. An additional portion of 50 μM cGMP was added to the preincubation mixture at 60 min after the start of preincubation as indicated by an arrow (open triangles, FIG. 11A). PDE5 activity was expressed as pmol/min/μg of protein. As shown in FIG. 11C, ³H-cGMP (total 100,000 cpm) was added to the preincubation mixture containing 50 μM cGMP without (filled squares), or with 0.2 μM sildenafil (open squares). The percent of 50 μM cGMP hydrolysis at different time points was measured using the PDE5 activity assay. After 5 min of preincubation with cGMP less than 0.01 μM cGMP was carried over to PDE5 assay mixture after all dilutions, and longer preincubations resulted in even less carry over of cGMP.

It was found that preincubation with cGMP led to a significant time-dependent reversible activation of PDE5 (FIG. 11A). Maximum activation of PDE5 (10 times) was observed after 15 min of preincubation. Longer preincubation led to a gradual decline of activated PDE5 activity. By 2 hrs of preincubation PDE5 activity was similar to the control samples. However, PDE5 activity could be reactivated after addition of another portion of cGMP to the preincubation mixture (FIG. 11A). Again, cGMP-induced activation was time-dependent with maximum activation comparable with a first activation step.

If activation of PDE5 depends on the presence of a certain level of cGMP during the preincubation step, it should be possible to prolong the activation time period by adding a PDE5 inhibitor to the preincubation mixture. Indeed, in the presence of increasing concentrations of sildenafil, PDE5 was active for a longer period of time. At 0.2 μM sildenafil PDE5 activation was sustained for at least 90 min (FIG. 11B). At this concentration of sildenafil the cGMP level declined much more slowly than without any PDE5 inhibitors (FIG. 11C). Apparently, decline in PDE5 activation coincided with complete hydrolysis of cGMP during the preincubation step. In all further experiments recombinant PDE5 was preincubated for 15 min with 50 μM cGMP in order to achieve maximum PDE5 activation.

Thus, a certain balance between the concentrations of PDE5, cGMP and duration of preincubation needed to be maintained in order to achieve saturation of the cGMP binding sites. For example, more concentrated PDE5 samples would most likely require a higher amount of CGMP and different preincubation time to get the highest level of activation.

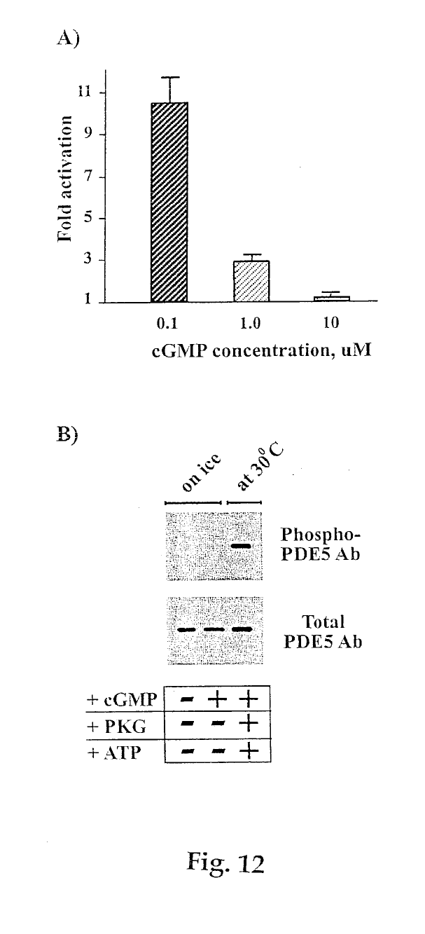

To investigate this further, PDE5 was preincubated of with 50 μM cGMP on ice and an aliquot was diluted and assayed with either 0.1 μM, 1.0 μM or 10 mM cGMP for 5 min at 30° C. Data are expressed as fold activation of PDE5 activity after preincubation with 50 μM cGMP (FIG. 12A). PDE5 activation was found to be the highest when PDE5 activity was assayed at 0.1 uM cGMP (10 times) as shown above. Activation was less at 1.0 μM cGMP (2-3 times) and not detectable when PDE5 activity was analyzed at 10 μM cGMP (FIG. 12A).

The Observed Activation of PDE5 is Not a Result of PDE5 Phosphorylation.

To explore the possibility that endogenous protein kinases from HEK 293 cells could phosphorylate and activate PDE5 during its preincubation with cGMP on ice, samples were prepared in SDS sample buffer before and after preincubation with 50 μM cGMP on ice and analyzed by Western blotting with phospho-specific PDE5 antibody and total PDE5 antibodies. As control for phospho-PDE5 the same amount of recombinant PDE5 was phosphorylated in the presence of ATP, PKG and cAMP for 30 min at 30° C. However, no PDE5 phosphorylation was detected after preincubation of PDE5 with 50 uM cGMP for 15 min on ice (FIG. 12B). Phosphorylation of PDE5 could be achieved only after addition of ATP, PKG and incubation at 30° C.

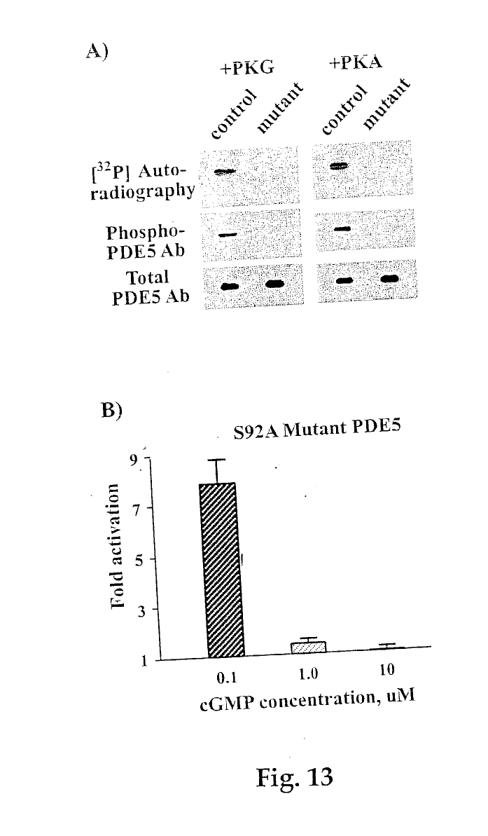

In order to test if PKG or PKA could phosphorylate another phosphorylation site, not detected by application of phospho-specific PDE5 antibody (raised against phospho-serine-92), site-directed mutagenesis was applied. Serine-92 was mutated to alanine, and recombinant PDE5 (control) and the phosho-site mutant PDE5 (mutant) were subjected to in vitro phosphorylation by PKG or the catalytic subunit of PKA in the phosphorylation buffer with ³²P-ATP for 60 min at 30° C. After phosphorylation PDE5 was immunoprecipitated, and the immunoprecipitates were analyzed by SDS-PAGE and then subjected to autoradiography to reveal [³²p] incorporation. For Western blot analysis samples were prepared directly after phosphorylation step. The immunoblots were probed with phospho-PDE5 and total PDE5 antibodies (FIG. 13A). Still, no ³²P incorporation was detected in the mutant PDE5 band (FIG. 13A), indicating that there is no other phosphorylation site besides serine 92. Moreover, when phospho-site mutant PDE5 was expressed in HEK 293 cells and assayed after preincubation with 50 μM cGMP on ice with either 0.1 μM, 1.0 μM or 10 μM cGMP for 5 min at 30° C., a similar pattern of PDE5 activation after preincubation with cGMP was found (FIG. 13B). The highest level of PDE5 activation was also observed when PDE5 activity was measured at low substrate concentration (0.1 μM cGMP). The data shows that the activation is not due to phosphorylation and therefore is due to direct activation by binding of the small molecule to the enzyme.

A mouse monoclonal antibody specifically blocks cGMP binding to the GAF A domain of PDE5. The previous studies showed that a short preincubation of PDE5 with cGMP on ice did not cause PDE5 phosphorylation, but was sufficient to induce PDE5 activation. To prove that the effect of PDE5 activation is due to the direct effect of cGMP occupancy of the cGMP-binding sites on the PDE5 catalytic activity, mouse monoclonal antibodies, generated against the cGMP-binding domain of PDE5, were developed and screened for their ability to affect cGMP binding.

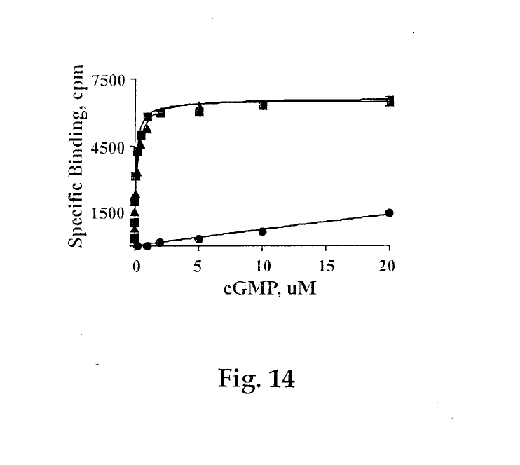

The cGMP saturation binding assay was employed (FIG. 14) using recombinant PDE5 without (filled squares) or with preincubation with monoclonal antibodies mAb/P3B2 (filled circles) or mAb/P4D4 (filled triangles) for 30 min on ice, to determine the Kd for cGMP binding. The binding assays were performed with 0.01-20 μM cGMP in the presence of 0.1 μM IBMX for 30 min on ice. The specific binding (cpm) is calculated by subtraction of nonspecific binding from total binding. Recombinant PDE5 was found to bind cGMP with high affinity (Kd=0.12 μM cGMP) (FIG. 14). A linear scatchard plot of the cGMP binding data indicated one class of cGMP-binding sites. All monoclonal antibodies were preincubated with PDE5 for 20 min followed by incubation with different concentrations of ³H-cGMP (0.01-20 μM cGMP). One monoclonal antibody (mAb/P3B2), was able to substantially block cGMP binding to PDE5 (FIG. 14). The apparent Kd for cGMP binding increased more than 100 times, but precise determination of Kd values at these high concentrations of cGMP (20-100 μM), was not possible using a Millipore filtration assay. Another monoclonal antibody (mAb/P4D8) used as a control did not have any effect on cGMP binding.

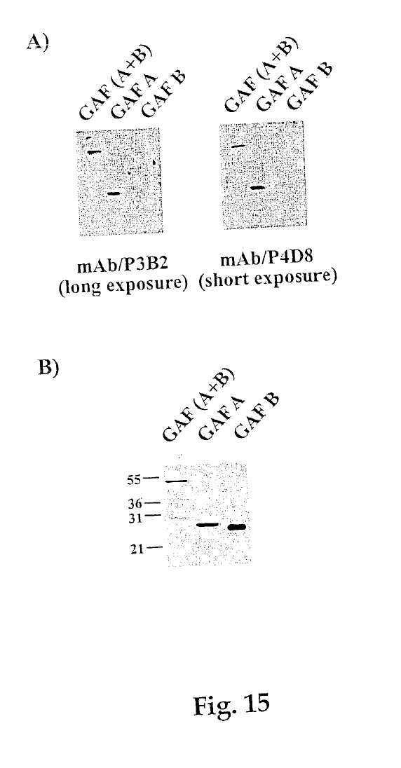

To determine which regions of cGMP binding domain contain the epitopes for these monoclonal antibodies, purified mouse GAF A, GAF B and the whole cGMP binding domain (GAF A and GAF B) were subjected to SDS-PAGE (12% (w/v) gel) and then either immunoblotted with either mAb/P3B2 or mAb/P4D8 (FIG. 15A), or analyzed by Silver staining (FIG. 15B). While mAb/P3B2 was much more effective in immunoprecipitation of PDE5 than in its detection by Western blotting, it was clear that mAb/P3B2 binds with GAF A, but not GAF B (FIG. 15A and B).

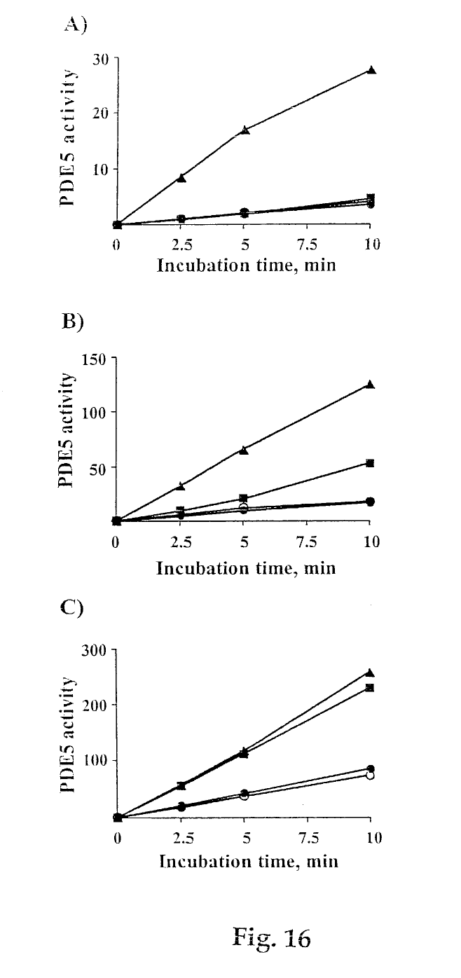

Blocking cGMP binding to the GAF A domain of PDE5 by a specific monoclonal antibody (mAb/P3B2) reveals a low intrinsic catalytic activity of PDE5. PDE5 activity was measured at 0.1 μM (FIG. 16A), 1.0 μM (FIG. 16B) or 10 μM cGMP (FIG. 16C). It was analyzed without any treatments (filled squares), or after preincubation with 50 μM cGMP on ice (filled triangles), or after preincubation with mAb/P3B2 for 30 min on ice (filled circles). Treatment of PDE5 with mAb/P3B2 for 30 min on ice preceded preincubation with 50 μM cGMP (open circles). PDE5 activity was expressed as μmol/pg of protein.

Treatment of PDE5 with a cGMP blocking monoclonal antibody (mAb/P3B2), before preincubation with cGMP not only completely blocked PDE5 activation, but also lowered PDE5 activity of control samples (FIG. 16). In all experiments, conducted at different substrate concentrations (0.1-10 μM cGMP), both activated PDE5 (after preincubation with cGMP) and control PDE5 (without preincubation with cGMP) reached the same low level of enzymatic activity. These data indicate that, by removing the activation effect of cGMP/GAF domain on PDE5 catalytic domain, PDE5 has a low intrinsic hydrolytic activity.

Interestingly, changes were not detected in PDE5 activity after preincubation with cGMP when PDE5 activity was assayed at 10 μM cGMP (FIG. 12A). However, by blocking cGMP binding with a specific monoclonal antibody (mAb/P3B2), PDE5 activity dropped 60-70% (FIG. 16C), indicating that, under these conditions, PDE5 was already activated. Apparently, 10 μM cGMP was sufficient to rapidly occupy all cGMP binding sites, and turn PDE5 into the activated state, even without preincubation with cGMP.

Activated PDE5 displayed linear kinetics of cGMP hydrolysis at least over 5 min of the incubation time at 0.1 μM cGMP and 10 min at 1.0 μM cGMP. Meanwhile, control (non-activated) PDE5, appeared to undergo activation at the end of 10 min of incubation, at both substrate concentrations (FIG. 16A and 16B).

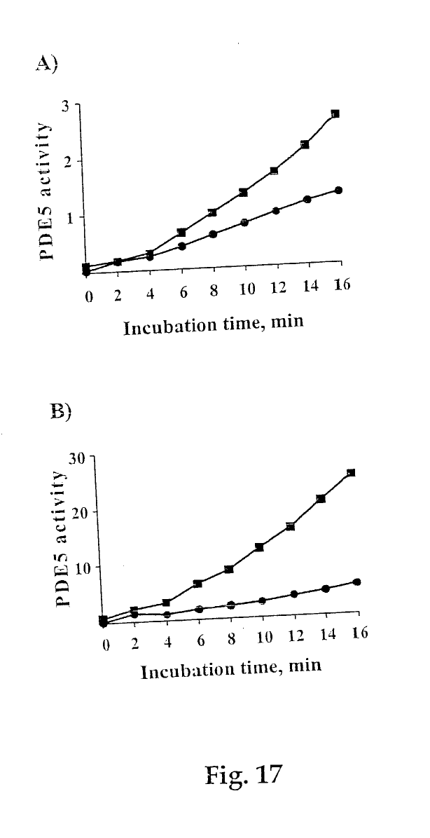

PDE5 is converted into the activated state at low substrate concentrations upon cGMP binding. To determine if PDE5 is activated during the assay, PDE5 activity was measured at 0.01 μM cGMP (FIG. 17A), 1.0 μM cGMP (FIG. 17B), at 30° C. without any treatment (filled squares), or after preincubation with mAb/P3B3 for 20 min on ice (filled circles). PDE5 activity was measured as pmol/μg of protein. In this assay cGMP hydrolysis was measured every 2 min and the incubation time was increased up to 16 min. A greater dilution of PDE5 was also necessary in order not to exceed 25% hydrolysis at the end of the activity assays.

At both substrate concentrations (0.1 μM and 1.0 μM cGMP) after a short lag period (4-6 min) activity of PDE5 gradually began to increase (FIG. 17A and B). Once again, blocking cGMP binding by mAb/P3B2 linearized the kinetics of cGMP hydrolysis at the lower rate. Since 0.1 μM cGMP was near the Kd for cGMP binding of this protein, only a part of the cGMP binding sites was expected to bind cGMP. At 1.0 μM Cgmp, more binding sites were occupied by cGMP, leading to the higher degree of PDE5 activation during the assay, and subsequently bigger reduction in total cGMP hydrolytic activity, after treatment with the blocking monoclonal antibody (FIG. 17B).

These experiments present another example of conversion of a low activity state enzyme to a high activity state upon cGMP binding. However, in contrast to PDE2 (Beavo, et al., (1971) J. Biol. Chem. 246:3841-3846; Martins, et al., (1982) J. Biol. Chem. 257:1973-1979; Charbonneau, et al., (1990) Proc. Nat. Acad. Sci. USA 87:288-292; Le Trong, et al., (1990) Biochemistty 29:10280-10288) PDE5 needs a transitional period of several minutes to reach the binding equilibrium at these low cGMP concentrations with cGMP binding sites on GAF A of PDE5 at 30° C.

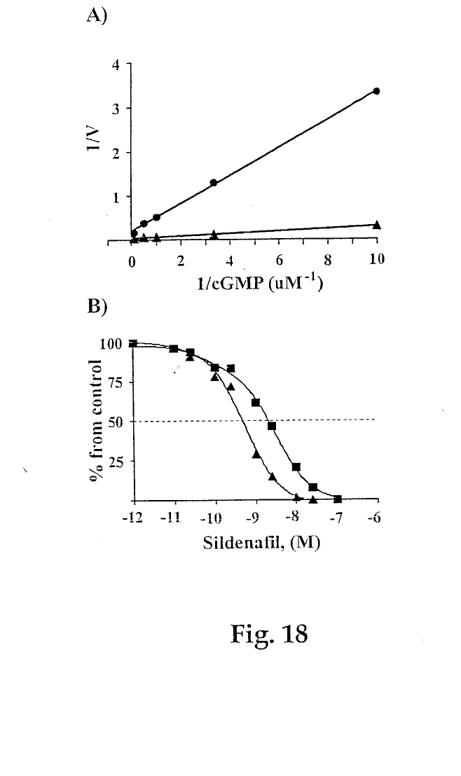

Kinetic and inhibitory characteristics of activated PDE5. At first, changes in PDE5 activity were detected after preincubation with cGMP only when PDE5 activity was assayed at 0.1 and 1.0 μm, but not at 10 μm (FIG. 12A). These data suggested that activation affected the apparent value of Km, without any changes in the Vmax value for PDE5. However, the experiments with the mAb/P3B2 monoclonal antibody revealed that a significant portion of PDE5 activity at 10 μm cGMP was already activated. Since activation of PDE5 was time and concentration dependent, in order to determine the kinetic characteristics of PDE5, the enzyme was treated with the monoclonal antibody and used as a non-activated enzyme, or the enzyme was used after preincubation with 50 μM cGMP on ice, as fully activated. FIG. 18A depicts a Lineweaver-Burk double-reciprocal plot (0.1-10 μM cGMP) of activated (after preincubation with cGMP on ice) (filled triangles) PDE5 and mAb/P3B2 treated PDE5 (filled circles). PDE5 activity was expressed as pmol/min/μg of protein. Control (without any preincubations) (filled squares) and activated (after preincubation with cGMP on ice) (filled triangles) samples of PDE5 were assayed for 5 min at 0.1 μM in the presence of different concentrations of sildenafil (0.01-250 μM) (FIG. 18B). PDE5 activities, assayed at each substrate concentration without sildenafil, were expressed as 100%. Km, Vmax and IC₅₀ values were calculated using GraphPad Prism 2.0 C (GraphPad Software, San Diego, Calif.).

As revealed by a Lineweaver-Burk analysis, activation of PDE5 altered both the Km and the Vmax (FIG. 18A). Activated PDE5 had a higher affinity for cGMP (the Km changed from 4.6 μM to 0.96 μM) and a higher Vmax value (the Vmax changed from 8.9 to 27 pmol/min/μg of protein).

Activated PDE5 also showed higher sensitivity towards the PDE5 specific inhibitor sildenafil. We found a shift in the IC₅₀ for sildenafil inhibition from 2.1 nM to 0.6 nM when PDE5 activity was assayed at 0.1 μM cGMP (FIG. 18B). At 1.0 μM cGMP the IC₅₀ decreased from 4.9 nM to 1.2 nM. Since sildenafil is considered a competitive inhibitor, which binds at the catalytic site of PDE5 (Ballard et al., 1998 J Urol, 159, 2164-2171), the different sildenafil inhibitory profile for activated PDE5 presumably reflects changes in kinetic characteristics caused by cGMP induced PDE5 activation. These data also demonstrate that cGMP binding to the GAF domain affects the catalytic domain of PDE5.

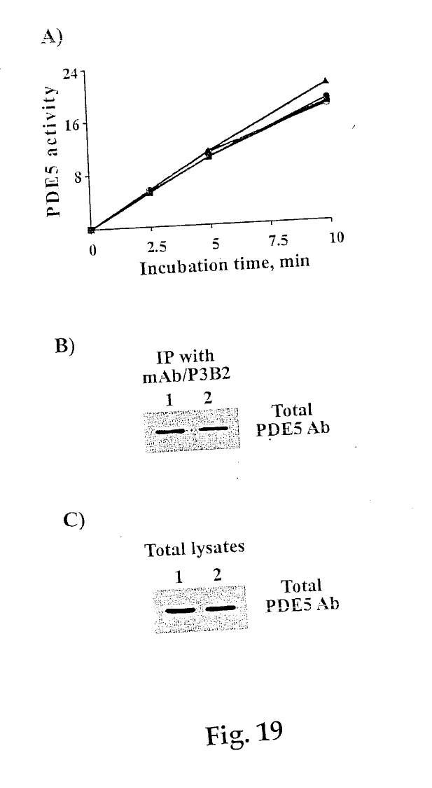

PDE5 gets activated and losses its ability for cGMP stimulation after 1-2 weeks of storage on ice. PDE5 activity was measured with 0.1 μM after 2 weeks of storage on ice (filled squares). Samples were prepared under the same conditions as in FIG. 16 and preincubated with cGMP (filled triangles), mAb/P3B2 (filled circles) or both of them (open circles). PDE5 activity was expressed as pmol/[g of protein (FIG. 19A). Samples of immunoprecipitates with mAb/P3B2 (FIG. 19B) and the total lysates (FIG. 19C) of freshly expressed (FIG. 19B and C, lane 1) or 2 weeks stored on ice (FIG. 19B and C, lane 2) cell lysates were prepared in SDS sample buffer and analyzed by Western blotting with total PDE5 antibody.

All previously described experiments were performed on recombinant PDE5 within a week of cell harvesting. Nevertheless, gradually after a week of storage on ice the basal PDE5 activity got higher and its response to preincubation with cGMP became weaker. By about 2 weeks the cGMP hydrolyzing activity of PDE5 reached approximately the same level as activated PDE5 (FIG. 16A), and completely lost its responsiveness to cGMP stimulation (FIG. 19A). At the same time the effect of mAb/P3B2 on PDE5 catalytic activity also ended, although this monoclonal antibody was still able to immunoprecipitate PDE5 (FIG. 19B).

Since no changes in the amount of total PDE5 or in the immunoprecipitation pattern with mAb/P3B2 were found between freshly expressed PDE5 preparations, or preparations stored on ice for 2 weeks stored on ice PDE5 preparations, proteolytic degradation of PDE5 could be ruled out as the cause of slow activation (FIG. 19C). This slow activation may explain, however, why PDE5 activation by cGMP has not been seen previously.

Discussion

Activation of PDE5 by cGMP binding to the GAF A domain is a fundamental feature of this enzyme. These results show that PDE5 can be directly activated by cGMP binding even at low, physiological substrate concentrations (0.1 μM and 1.0 μM CGMP). In order to detect full activation of PDE5 at low substrate concentrations a preincubation step is required. Blocking cGMP binding by the application of the mouse monoclonal antibody (mAb/P3B2) allowed quantitative evaluation of the effect of occupancy of cGMP binding sites on total cGMP hydrolyzing activity of PDE5. Pretreatment of PDE5 with this blocking antibody linearized the kinetics of cGMP hydrolysis over time, substantially lowering PDE5 activity. If the cGMP binding sites were saturated with cGMP, which was achieved after a short preincubation with 50 μM cGMP on ice, activated PDE5 showed a linear kinetic profile of cGMP hydrolysis, and blocking cGMP binding with mAb/P3B2 completely prevented PDE5 from activation. These data strongly indicate that PDE5 in a non-activated state possesses a low intrinsic catalytic activity if the stimulatory effect of the cGMP/GAF domain is blocked.

cGMP binding to the GAF domain of PDE5 is also a prerequisite for PDE5 phosphorylation. Recently, it has been shown that PDE5 is a specific substrate for PKG in intact smooth muscle cells and tissues (Rybalkin et al., 2002 J Biol Chem, 277, 3310-3317). Thus, activation of both PDE5 and PKG by cGMP would depend on their cGMP binding characteristics in vivo. While not wishing to be bound by any theory, activation of PDE5 by direct cGMP binding may present a rapid response to a burst of cGMP inside a cell, limiting the amplitude and duration of cGMP at the sub-micromolar level. Further accumulation of cGMP would eventually activate PKG, leading to PDE5 phosphorylation and longer lasting activation, thereby initiating a longer term cycle of negative feedback regulation of the cGMP signal.

PDE5 activity appears to be a decisive factor in controlling cGMP signaling in cells other than smooth muscle as well. For example, in platelets, activation and phosphorylation of PDE5 was found to be responsible for a rapid decline in cGMP level after NO stimulation, whereas neither guanylyl cyclase or other PDEs were involved (Mullershausen et al., (2001) J Cell Biol, 155, 271-278).

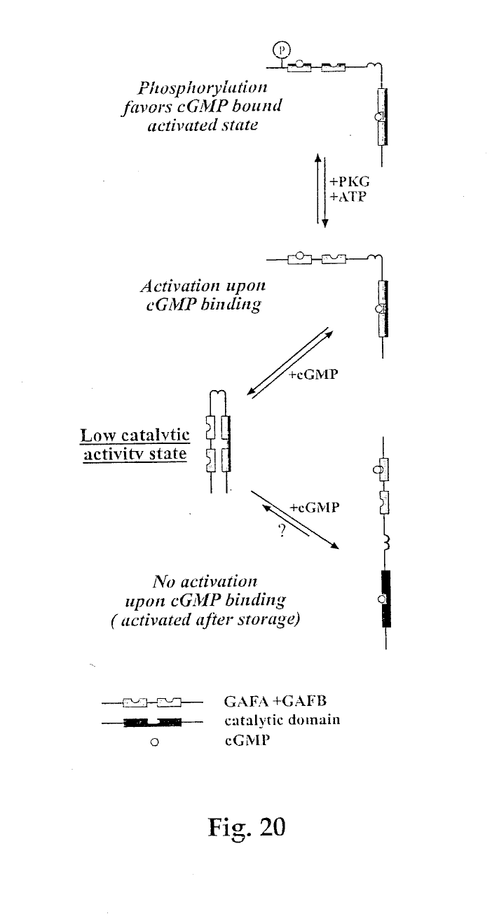

Different conformational states of PDE5. These results also suggest that PDE5 can exist in at least three different conformational states: non-activated, activated by cGMP and activated after storage (FIG. 20). A non-activated low intrinsic catalytic activity state and cGMP activated states are two reversible conformational states of PDE5 with different kinetic and inhibitory properties. It is likely that these are the “native states”, which are present in vivo, and respond to fluctuations in cGMP levels, via cGMP induced allosteric transition, from the low catalytic activity state to the activated state.

The ability of PDE5 to be directly activated by cGMP was limited to relatively fresh preparations (less than a week after harvesting transfected cells). Longer storage resulted in a complete loss of the cGMP/GAF domain effect on the catalytic activity of PDE5. At the same time these changes might explain the absence of cGMP effect on the purified PDE5 catalytic activity in earlier reports, when purification used to take many days (Thomas et al., 1990 J Biol Chem, 265, 14964-14970). Apparently, PDE5 is more susceptible to storage or purification conditions than PDE2, which can also show less response to cGMP stimulation, but only after a much longer storage. Thus, PDE5 activated after storage reveals an “artificial”, probably irreversible, conformational state of PDE5, suggesting that interaction between the cGMP/GAF domain and the catalytic domain has been interrupted (FIG. 20). Thus, surprisingly, stored PDE5, which represents an artificially activated form of PDE5, is unsuitable for screening for activators.

Recently, it has been reported that the hydrolysis of a fluorescent analog of cGMP (Ant-cGMP—2′-O-anthranyloyl cyclic GMP) by PDE 5 could exhibit positive cooperativity at a few concentrations of cGMP, suggesting the possibility of direct activation (Okada and Asakawa, 2002 Biochemistry, 41, 9672-9679). However, under most conditions, only inhibition due to substrate competition was observed. It is quite possible that this modest effect could be due to the aged nature of the enzyme preparation used by these investigators. Nonetheless, earlier observation of stimulation of Ant-cGMP hydrolysis in intact cells (Hartell et al., 2001 Neuroreport, 12, 25-28) is consistent with the direct activation caused by cGMP binding to the GAF domain, as shown herein.

PDE5 and other GAF domain containing proteins. Recently, an electron microscopy study of PDE5 and PDE6, that were purified to homogeneity, showed a remarkable similarity in their molecular organization (Kameni Tcheudji et al., 2001 J Mol Biol, 310, 781-791). Both enzymes had three electron-dense subdomains, including the catalytic domain and two GAF domains, which were organized linearly from the C terminal to the N terminal end. The only contact points were found between the corresponding GAF domains in the PDE5 dimeric structure. However, a linear organization of PDE5 does not easily explain the effect of cGMP binding on PDE5 hydrolytic activity, since, there is no interaction between the cGMP binding GAF A domain and the catalytic domain. It is possible that the human platelet PDE5 used in that study after a long multi-step purification process is similar to a conformational state of PDE5 observed in our study after 2 weeks of storage (FIGS. 19 and 20).

Based on similar electron microscopy data for PDE5 and PDE6 it is reasonable to suggest that PDE6 may also exist in the same conformational state as PDE5 and cGMP binding to the PDE6 GAF domain may have a regulatory effect on the catalytic domain of PDE6 in its “native” state. So far only the interaction between the gamma-subunit and the catalytic domain of PDE6 has been shown to be affected by cGMP binding to its GAF domain (Norton et al., 2000; Yamazaki et al., 1980 J Biol Chem, 275, 38611-38619).

Moreover, the mechanism of the GAF domain stimulatory effect on the catalytic domain might be extended not only to other cGMP binding PDEs, but to other GAF domain containing proteins as well. Another confirmation of interaction between these two domains is provided by a study of cAMP-activated adenylyl cyclase from cyanobacteria (Kanacher et al., 2002 Embo J, 21, 3672-3680). After replacing the cAMP binding GAF domain of cyanobacteria with the cGMP binding GAF domain from rat cGMP-stimulated PDE (PDE2), the chimeric protein became responsive to cGMP stimulation with the same EC50 as for regular PDE2, suggesting that despite a wide evolutionary gap the basic mechanism of the GAF domain effect on the catalytic activity remained the same.

These results, taken together with the results obtained herein, suggest that the mechanism of the GAF domain interaction with the catalytic domain may be generally applicable to many GAF domain containing proteins, such as PDE2. Other unidentified small molecules should perform the same way as cGMP/or cAMP, in this diverse family of GAF domain containing proteins, and may be useful for functional modulation of GAF domain containing proteins.

Interestingly, PDE5 activity after 2 weeks of storage on ice could reach levels comparable with the levels of freshly expressed PDE5 after full activation by cGMP (compare FIG. 16A and FIG. 19A), suggesting a possible mechanism of cGMP induced allosteric transformation of PDE5. This mechanism would include physical interaction between the GAF domain and the catalytic domain. In the absence of CGMP when PDE5 is in a “native” state, the GAF domain physically blocks the catalytic domain resulting in a low intrinsic catalytic activity. cGMP binding relieves the inhibitory effect and the catalytic domain becomes open. In PDE5 after storage, all intramolecular domains acquire linear organization, and the catalytic domain stays open and constitutively active. This type of activation has been reported for other proteins including the Epac family and guanine nucleotide exchange factors that are directly activated by cAMP (de Rooij et al., 2000 J Biol Chem, 275, 20829-20836). In this case the cAiMP binding domain suppresses the catalytic activities of Epac1 and Epac2, and cAMP binding results in a release of such inhibition of the catalytic domain of Epac proteins.

The GAF A domain of PDE5 as a potential target for the development of new PDE5 inhibitors. All known PDE inhibitors were developed to compete with cGMP (or cAMP) at the catalytic site. Since PDEs from different families share a certain degree of sequence similarity in their catalytic domain, inhibitory profiles of specific PDE inhibitors towards PDEs from different families partially overlap. For example, PDE5 and PDE6 have a very high percent identity in their catalytic domain, and sildenafil can inhibit PDE6 with an IC₅₀ only a little higher than PDE5, but still in the nM range of concentrations. As a result, this drug may produce visual side effects in clinical applications due to its inhibitory effect on PDE6 in photoreceptor cells.

These results present evidence of direct activation of PDE5 upon cGMP binding to the GAF A domain as a new fundamental mechanism of regulation of PDE5 activity. This mechanism also suggests that the cGMP binding sites on the GAF domains are good new targets for development of both agonists and antagonists of PDE activity. The data also suggest that the mechanism of enzyme alteration induced by ligand binding to the GAF domain may be common for other GAF domain containing proteins.

Various publications are cited herein that are hereby incorporated by reference in their entirety.

As will be apparent to those skilled in the art to which the invention pertains, the present invention may be embodied in forms other than those specifically disclosed above without departing from the spirit or essential characteristics of the invention. The particular embodiments of the invention described above, are, therefore, to be considered as illustrative and not restrictive. The scope of the present invention is as set forth in the appended claims rather than being limited to the examples contained in the foregoing description.

What is claimed is:

1. A method for modulating the enzymatic activity of PDE5, comprising contacting PDE5 with an effective amount of an agent that binds PDE5 and activates or inhibits PDE5.

2. The method of claim 1 wherein the agent binds at an allosteric or catalytic site of PDE5.

3. The method of claim 2, wherein the allosteric site is a cGMP-binding domain.

4. The method of claim 2, wherein the allosteric site is a GAF domain.

5. The method of claim 4, wherein the GAF domain is a GAF A or GAF B domain.

6. The method of claim 2 wherein the agent binds to the allosteric site and activates PDE5.

7. The method of claim 2 wherein the agent binds to the allosteric site and inhibits PDE5.

8. The method of claim 2 wherein the agent binds to the catalytic site and inhibits PDE5.

9. The method of claim 2 wherein the agent binds to both the allosteric and catalytic sites of PDE5.

10. The method of claim 1 wherein the agent that binds PDE5 is selected from the group consisting of antibodies, peptides, proteins, oligonucleotides, antisense DNA and RNA, small interfering RNAs (siRNAs), non-peptide compounds, and small inorganic or organic molecules.

11. The method of claim 10, wherein the agent that binds PDE5 is an anti-PDE5 antibody.

12. The method of claim 10, wherein the agent that binds PDE5 is a small molecule selected from the group consisting of sildenafil, tadalafil, tildenafil, vardenafit, analogs thereof, and cGMP analogs.

13. A method for identifying an agent that specifically binds to PDE5 comprising:
a) contacting PDE5 with an effective amount of a test agent; and
b) determining if the test agent specifically binds PDE5.

14. The method of claim 13 wherein step (b) comprises determining if the test agent specifically binds to an allosteric or catalytic site of PDE5.

15. A method for identifying an agent that specifically binds to PDE5 so as to modulate the enzymatic activity of PDE5 comprising:
a) contacting PDE5 with an effective amount of a test agent;
b) determining if the test agent specifically binds PDE5; and
c) testing for activation or inhibition of PDE5 activity.

16. The method of claim 14 or 15, wherein the PDE5 is recombinant PDE5.

17. The agent identified by the method of claim 15, wherein the agent binds to the catalytic site and is an inhibitor of the PDE5.

18. The agent identified by the method of claim 15, wherein the agent binds to an allosteric site and is an activator of the PDE5.

19. The agent identified by the method of claim 15, wherein the agent binds to an allosteric site and is an inhibitor of the PDE5.

20. The method of claim 15, wherein the agent is selected from the group consisting of antibodies, peptides, proteins, oligonucleotides, small interfering RNAs (siRNA), non-peptide compounds, and small inorganic or organic molecules.

21. A pharmaceutical composition comprising the agent identified by any of the methods of claims 13-20 and a pharmaceutically acceptable carrier.

22. A method of using an agent that modulates the enzymatic activity of PDE5 in a pharmaceutical composition for treating a subject for a condition where inhibition of PDE5 is of therapeutic benefit, comprising administering to said subject a therapeutically effective amount of the pharmaceutical composition so as to inhibit PDE5.

23. A method of using an agent that modulates the enzymatic activity of PDE5 in a pharmaceutical composition for treating a subject for a condition where activation of PDE5 is of therapeutic benefit, comprising administering to said subject a therapeutically effective amount of the pharmaceutical composition so as to activate PDE5.

24. The method of claim 22 or 23 wherein the subject has a condition that responds to a vasodilatory agent.

25. The method of claim 22 or 23 wherein the condition is pulmonary arterial hypertension.

26. The method of claim 22 or 23 wherein the condition is erectile dysfuinction in a subject.

27. The method of claim 22 or 23, wherein the subject is a human, non-human primate, farm animal, household pet, experimental animal, or an animal in captivity.

28. The method of claim 22 or 23, wherein the treatment is by intravenous injection, intramuscular injection, subcutaneous injection, implantable pump, continuous infusion, gene therapy, liposomes, biodegradable polymers, hydrogels, oral administration, or controlled release patch.

29. A method of treating stable angina, unstable angina, variant angina, hypertension, pulmonary hypertension, pulmonary arterial hypertension, chronic obstructive pulmonary disease, acute respiratory distress syndrome, malignant hypertension, pheochromocytoma, congestive heart failure, acute renal failure, chronic renal failure, atherosclerosis, a condition of reduced blood vessel patency, a peripheral vascular disease, a vascular disorder, thrombocythemia, an inflammatory disease, myocardial infarction, stroke, bronchitis, chronic asthma, allergic asthma, allergic rhinitis, glaucoma, peptic ulcer, a gut motility disorder, postpercutaneous transluminal coronary or carotid angioplasty, post-bypass surgery graft stenosis, osteoporosis, preterm labor, benign prostatic hypertrophy, irritable bowel syndrome, peptic ulcer, diseases characterized by disorders of gut motility, appetite, depression, anxiety, motor function, memory, immune function, inflamnmation, autoimmune disease, amelioration of reperfusion injury, sepsis, hypotension, and reversal of nitrovasodilator overdose including an overdose of viagra in a human or nonhuman animal subject, said method comprising administering to said subject a therapeutically effective amount of a pharmaceutical composition of claim 21.

30. The method of claim 29 wherein the treatment is by intravenous injection, intramuscular injection, subcutaneous injection, implantable pump, continuous infusion, gene therapy, liposomes, biodegradable polymers, hydrogels, oral administration, or controlled release patch.


SEQUENCE LISTING (47 sequences)

SEQ ID NO: 1 - protein, 20 aa, bovine
  misc_feature (3)..(3): Xaa can be any naturally occurring amino acid
REXDANRINYMYAQYVKNTM

SEQ ID NO: 2 - protein, 9 aa, bovine
QSLAAAVVP

SEQ ID NO: 3 - protein, 8 aa, Artificial
  Peptide from Bovine PDE5
FDNDEGEQ

SEQ ID NO: 4 - DNA, 23 bp, Artificial Sequence
  Primer
ttygayaaygaygarggngarca

SEQ ID NO: 5 - DNA, 23 bp, Artificial Sequence
  Primer
aarctrttrctrctyccnctygt

SEQ ID NO: 6 - DNA, 23 bp, Artificial Sequence
  Primer
aaytayatgtaygcncartaygt

SEQ ID NO: 7 - DNA, 23 bp, Artificial Sequence
  Primer
ttratrtacatrcgngtyatrca

SEQ ID NO: 8 - DNA, 36 bp, Artificial Sequence
  Primer
ttratrtacatrcgngtyatrcanttyttrtgntac

SEQ ID NO: 9 - DNA, 4474 bp, bovine
  CDS (99)..(2723)
gggagggtctcgaggcgagttctgctcctcggagggagggaccccagctggagtggaaaaccagcaccagctgaccgcagagacacgccgcgctgatcatggagagggccggccccggctgccgcgcggccgcaacagcaatgggaccaggactcggtcgaagcgtggctggacgatcactgggactttaccttctctactttgttaggaaaggcaccagagaaatggtcaacgcatggtttgctgagagagttcacaccattcctgtgtgcaaggaaggaatcaagggccacacggaatcctgctcttgccccttgcagccaagtccccgtgcagagagcagtgtccctggaacaccaaccaggaagatctctgcctctgaattcgatcggccgcttagacccatcgttatcaaggattctgagggaactgtgagcttcctctctgactcagacaagaaggaacagatgcctctaacctccccacggtttgataatgatgaaggggaccagtgctcgagactcttggaattagtgaaagatatttctagtcacttggatgtcacagccttatgtcacaaaattttcttgcacatccatggactcatctccgccgaccgctactccttattcctcgtctgtgaggacagctccaacgacaagtttcttatcagccgcctctttgatgttgcagaaggttcaacactggaagaagcttcaaacaactgcatccgcttagagtggaacaaaggcatcgtgggacacgtggccgcttttggcgagcccttgaacatcaaagacgcctatgaggatcctcgattcaatgcagaagttgaccaaattacaggctacaagacacaaagtattctttgtatgccaattaagaatcatagggaagaggttgttggtgtagcccaggccatcaacaagaaatcaggaaatggtgggacattcactgaaaaagacgaaaaggactttgctgcttacttggcattttgtggaattgttcttcataatgctcaactctatgagacttcactgctggagaacaagagaaatcaggtgctgcttgaccttgctagcttaatttttgaagaacaacaatcattagaagtaattctaaagaaaatagctgccactattatctctttcatgcaggtgcagaaatgcaccattttcatagtggatgaagattgctccgattctttttctagtgtgtttcacatggagtgtgaggaattagaaaaatcgtcagatactttaacacgggaacgtgatgcaaacagaatcaattacatgtatgctcagtatgtcaaaaataccatggaaccacttaatatcccagacgtcagtaaggacaaaagatttccctggacaaatgaaaacatgggaaatataaaccagcagtgcattagaagtttgctttgtacacctataaaaaatggaaagaagaacaaagtgataggggtttgccaacttgttaataagatggaggaaaccactggcaaagttaaggctttcaaccgcaacgatgaacagtttctggaagctttcgtcatcttttgtggcttggggatccagaacacacagatgtacgaagcagtggagagagccatggccaagcaaatggtcacgttagaggttctgtcttatcatgcttcagctgcagaggaagaaaccagagagctgcagtccttagcggctgctgtggtaccatctgcccagacccttaaaatcactgacttcagcttcagcgactttgagctgtctgacctggaaacagcactgtgcacaatccggatgttcactgacctcaaccttgtgcagaacttccagatgaaacatgaggtcctttgcaagtggattttaagtgtgaagaagaactatcggaagaacgtcgcctatcataattggagacatgcctttaatacagctcagtgcatgtttgcggcactaaaagcaggcaaaattcagaagaggctgacggacctggagatacttgcactgctgattgctgccttaagccatgatctggatcaccgtggtgtcaataactcatacatacagcgaagtgaacacccacttgctcagctctactgccattcaatcatggagcatcatcattttgatcagtgcctgatgatccttaatagtcctggcaatcagattctcagtggcctctccattgaagagtataagaccaccctgaagatcatcaagcaagctattttagccacagacctagcactgtacataaagagacgaggagaattttttgaacttataatgaaaaatcaattcaatttggaagatcctcatcaaaaggagttgtttttagcgatgctgatgacagcttgtgatctttctgcaattacaaaaccctggcctattcaacaacggatagcagaacttgttgccactgaattttttgaccaaggagatagagagaggaaagaactcaacatagagcccgctgatctaatgaaccgggagaagaaaaacaaaatcccaagtatgcaagttggattcatagatgccatctgcttgcaactgtatgaggccttgacccatgtgtcggaggactgtttccctttgctggacggctgcagaaagaacaggcagaaatggcaggctcttgcagaacagcaggagaagacactgatcaatggtgaaagcagccagaccaaccgacagcaacggaattccgttgctgtcgggacagtgtagccaggtgtatcagatgagtgagtgtgtgctcagctcagtcctctgcaacaccatgaagctaggcattccagcttaattcctgcagttgactttaaaaaactggcataaagcactagtcagcatctagttctagcttgaccagtgaagagtagaacaccaccacagtcagggtgcagagcagttggcagtctcctttcgaacccagactggtgaatttaaagaagagcagtcgtcgtttatatctctgtcttttcctaagcggggtgtggaatctctaagaggagagagagatctggaccacaggtccaatgcgctctgtcctctcagctgcttcccccactgtgctgtgacctctcaatctgagaaacgtgtaaggaaggtttcagcgaattccctttaaaatgtgtcagacagtagcttcttgggccgggttgttcccgcagctccccatctgtttgttgtctatcttggctgaaagaggctttgctgtacctgccacactctcctggatccctgtccagtagctgatcaaaaaaaaggatgtgaaattctcgtgtgactttttagaaaaggaaagtgaccccgaggatcggtgtggattcactagttgtccacagatgatctgtttagtttctagaattttccaagatgatacactcctccctagtctaggggtcagaccctgtatggtggctgtgacccttgaggaacttctctctttgcatgacattagccatagaactgttcttgtccaaatacacagctcatatgcagcttgcaggaaacactttaaaaacacaactatcacctatgttattctgattacagaagttatccctactcactgtaaacataaacaaagccccccaaacttcaaatagttgtgtgtggtgagaaactgcaagttttcatctccagagatagctataggtaataagtgggatgtttctgaaacttttaaaaataatcttttacatatatgttaactgttttcttatgagcactatggtttgtttttttttttttttgctctgctttgacttgcccttttcactcaattatcttggcagtttttctaaatgacttgcacagacttctcctgtacttcatggctgtgcagtgttccatgctgtgagggcaccatcgtgtattaaatcagttccctggtcacacataggtgagctggttggaaatttttaccattaaaaaaccactttcccacattgatgctttctaatctggcacaggatgcttctttttttcccctttttctctgtttaattattggaaatgggatctgtgggatcctcgttccctggcacctagctgctctcaacgtggcctgtggccagcagcattggctagacctgggggcttgttgggaacggagaccctctgccctgcccctggcctgctgacaaggacctgcattttgctgagctcccagtgaccctggtgtttaattgttaaccattgaaaaaaatcaaactatagtttatttacaatgttgtgttaatttcgggtgtacagcaaagtgactcagtggtcaagtacatttaaaacactgggcatactctctccctctccttgtgtacctggttggtatttccagaaaccatgctcttgtctgtcctgtagttttggaagcgctttctctttgaagactgccttctctcctctgtctgccctacatggactagttcgtttattgtcctacatggctttgcttccatgttcctctcaacttt

SEQ ID NO: 10 - protein, 875 aa, bovine
MERAGPGCRAAATAMGPGLGRSVAGRSLGLYLLYFVRKGTREMVNAWFAERVHTIPVCKEGIKGHTESCSCPLQPSPRAESSVPGTPTRKISASEFDRPLRPIVIKDSEGTVSFLSDSDKKEQMPLTSPRFDNDEGDQCSRLLELVKDISSHLDVTALCHKIFLHIHGLISADRYSLFLVCEDSSNDKFLISRLFDVAEGSTLEEASNNCIRLEWNKGIVGHVAAFGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTRERDANRINYMYAQYVKNTMEPLNIPDVSKDKRFPWTNENMGNINQQCIRSLLCTPIKNGKKNKVIGVCQLVNKMEETTGKVKAFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCKWILSVKKNYRKNVAYHNWRHAFNTAQCMFAALKAGKIQKRLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIMKNQFNLEDPHQKELFLAMLMTACDLSAITKPWPIQQRIAELVATEFFDQGDRERKELNIEPADLMNREKKNKIPSMQVGFIDAICLQLYEALTHVSEDCFPLLDGCRKNRQKWQALAEQQEKTLINGESSQTNRQQRNSVAVGTV

SEQ ID NO: 11 - DNA, 2060 bp, human cDNA
gcggccgcgctccggccgctttgtcgaaagccggcccgactggagcaggacgaagggggagggtctcgaggccgagtcctgttcttctgagggacggaccccagctggggtggaaaagcagtaccagagagcctccgaggcgcgcggtgccaaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgtccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccactttatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcag

SEQ ID NO: 12 - DNA, 1982 bp, human cDNA
acaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgag

SEQ ID NO: 13 - DNA, 18 bp, Artificial Sequence
  Primer
gccaccagagaaatggtc

SEQ ID NO: 14 - DNA, 18 bp, Artificial Sequence
  Primer
acaatgggtctaagaggc

SEQ ID NO: 15 - DNA, 18 bp, Artificial Sequence
  Primer
tcagtgcatgtttgctgc

SEQ ID NO: 16 - DNA, 18 bp, Artificial Sequence
  Primer
tacaaacatgttcatcag

SEQ ID NO: 17 - DNA, 1107 bp, human cDNA
gagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttgggg

SEQ ID NO: 18 - DNA, 18 bp, Artificial Sequence
  Primer
tttggaagatcctcatca

SEQ ID NO: 19 - DNA, 28 bp, Artificial Sequence
  Primer
atgtctcgagtcagttccgcttggcctg

SEQ ID NO: 20 - DNA, 30 bp, Artificial Sequence
  Primer
tacagaattctgaccatggagcgggccggc

SEQ ID NO: 21 - DNA, 18 bp, Artificial Sequence
  Primer
cattctaagcggatacag

SEQ ID NO: 22 - DNA, 2645 bp, human cDNA
  CDS (12)..(2636)
gaattctgaccatggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgtccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgactcgag

SEQ ID NO: 23 - protein, 875 aa, human cDNA
MERAGPSFGQQRQQQQPQQQKQQQRDQDSVEAWLDDHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 24 - protein, 235 aa, bovine
FQMKHEVLCKWILSVKKNYRKNVAYHNWRHAFNTAQCMFAALKAGKIQKRLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIMKNQFNLEDPHQKELFLAMLMTACDLSAITKPWPIQQRIAELVATEFFDQGDRERKELNIEPADLMNREKKNKIPSMQVGFID

SEQ ID NO: 25 - protein, 244 aa, bovine
FHIPQEALVRFMYSLSKGYRRITYHNWRHGFNVGQTMFSLLVTGKLKRYFTDLEALAMVTAAFCHDIDHRGTNNLYQMKSQNPLAKLHGSSILERHHLEFGKTLLRDESLNIFQNLNRRQHEHAIHMMDIAIIATDLALYCKKRTMFQKIVDQSKTYETQQEWTQYMMLDQTRKEIVMAMMMTACDLSAITKPWEVQSKVALLVAAEFWEQGDLERTVLQQNPIPMMDRNKADELPKLQVGFID

SEQ ID NO: 26 - protein, 244 aa, bovine
FQIPQEVLVRFLFSVSKGYRRITYHNWRHGFNVAQTMFTLLMTGKLKSYYTDLEAFAMVTAGLCHDIDHRGTNNLYQMKSQNPLAKLHGSSILERHHLEFGKFLLSEETLNIYQNLNRRQHEHVIHLMDIAIIATDLALYFKKRTMFQKIVDESKNYEDRKSWVEYLSLETTRKEIVMAMMMTACDLSAITKPWEVQSKVALLVAAEFWEQGDLERTVLDQQPIPMMDRNKAAELPKLQVGFID

SEQ ID NO: 27 - protein, 244 aa, bovine
FKVPVEVLTRWMTYVRKGYRAVTYHNWRHGFNVGQTMFTLLMTGRLKKYYTDLEAFAMLAAAFCHDIDHRGTNNLYQMKSTSPLARLHGSSILERHHLEYSKTLLQDESLNIFQNLNKRQYETVIHLFEVAIIATDLALYFKKRTMFQKIVDACEKMETEEEAIKYVTIDPTKKEIIMAMMMTACDLSAITKPWEVQSQVALLVANEFWEQGDLERTVLQQQPIPMMDRNKKDELPKLQVGFID

SEQ ID NO: 28 - protein, 233 aa, bovine
YKIDCPTLARFCLMVKKGYRDPPYHNWMHAFSVSHFCYLLYKNLELTNYLEDMEIFALFISCMCHDLDHRGTNNSFQVASKSVLAALYSSEGSVMERHHFAQAIAILNTHGCNIFDHFSRKDYQRMLDLMRDIILATDLAHHLRIFKDLQKMAEVGYDRTNKQHHSLLLCLLMTSCDLSDQTKGWKTTRKIAELIYKEFFSQGDLEKAMGNRPMEMMDREKAYIPELQISFME

SEQ ID NO: 29 - protein, 230 aa, bovine
FKIPVSCLIAFAEALEVGYSKYKNPYHNLIHAADVTQTVHYIMLHTGIMHWLTELEILAMVFAAAIHDYEHIGTTNNFHIQTRSDVAILYNDRSVLENHHVSAAYRLMQEEEMNVLINLSKDDWRDLRNLVIEMVLSTDMSGHFQQIKNIRNSLQQPEGLDKAKTMSLILHAADISHPAKSWKLHHRWTMALMEEFFLQGDKEAELGLPFSPLCDRKSTMVAQSQIGFID

SEQ ID NO: 30 - protein, 230 aa, bovine
FKIPTVFLMTFLDALETGYGKYKNPYHNQIHAADVTQTVHCFLLRTGMVHCLSEIEVLAIIFAAAIHDYEHIGTTNSFHIQTKSEQAILYNDRSVLENHHISSVFRMMQDDEMNIFINLTKDEFVELRALVIEMVLATDMSCHFQQVKSMKTALQQLERIDKSKALSLLLHAADISHPTKQWSVHSRWTKALMEEFFRQGDKEAELGLPFSPLCDRTSTLVAQSQIGFID

SEQ ID NO: 31 - protein, 240 aa, rat
FQIPADTLLRYLLTLEGHYHSNVAYHNSIHAADVVQSAHVLLGTPALEAVFTDLEVLAAIFACAIHDVDHPGVSNQFLINTNSELALMYNDSSVLENHHLAVGFKLLQGENCDIFQNLSTKQKLSLRRMVIDMVLATDMSKHMSLLADLKTMVETKKVTSLGVLLLDNYSDRIQVLQSLVHCADLSNPAKPLPLYRQWTERIMAEFFQQGDRERESGLDISPMCDKHTASVEKSQVGFID

SEQ ID NO: 32 - protein, 239 aa, drosophila
MIPPKTFLNFMSTLEDHYVKDNPFHNSLHAADVTQSTNVLLNTPALEGVFTPLEVGGALFAACIHDVDHPGLTNQFLVNSSSELALMYNDESVLENHHLAVAFKLLQNQGCDIFCNMQKKQRQTLRKMVIDIVLSTDMSKHMSLLADLKTMVETKKVAGSGVLLLDNYTDRIQVLENLVHCADLSNPTKPLPLYKRWVALLMEEFFLQGDKERESGMDISPMCDRHNATIEKSQVGFID

SEQ ID NO: 33 - protein, 385 aa, bovine
LLELVKDISSHLDVTALCHKIFLHIHGLISADRYSLFLVCEDSSNDKFLISRLFDVAEGSTLEEASNNCIRLEWNKGIVGHVAAFGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTRERDANRINYMYAQYVKNTMEPLNIPDVSKDKRFPWTNENMGNINQQCIRSLLCTPIKNGKKNKVIGVCQLVNKMEETTGKVKAFNRNDEQFLEAFVIFCGLGIQNTQMYEAVERAMAKQMVTLEVLSYHASAAEEE

SEQ ID NO: 34 - protein, 353 aa, bovine
ILQLCGELYDLDASSLQLKVLQYLQQETQASRCCLLLVSEDNLQLSCKVIGDKVLEEEISFPLTTGRLGQVVEDKKSIQLKDLTSEDMQQLQSMLGCEVQAMLCVPVISRATDQVVALACAFNKLGGDLFTDQDEHVIQHCFHYTSTVLTSTLAFQKEQKLKCECQALLQVAKNLFTHLDDVSVLLQEIITEARNLSNAEICSVFLIDQNELVAKVFDGGVLEDESYEIRIPADQGIAGHVATTGQILNIPDAYAHPLFYRGVDDSTGRFTRNILCFPIKNENQEVIGVAELVNKINGPWFSKFDEDLATAFSIYCGISIAHSLLYKKVNEAQYRSHLANEMMMYHMKVSDDE

SEQ ID NO: 35 - protein, 402 aa, bovine
LLEVLLEEAGSVELAAHRALQRLAQLLQADRCSMFLCRARNGTPEVASKLLDVTPTSKFEDNLVVPDREAVFPLDVGIVGWVAHTKKTFNVPDVKKNSHFSDFMDKQTGYVTRNLLATPIVMGKEVLAVFMAVNKVDASEFSKQDEEVFSKYLSFVSIILKLHHTNYLYNIESRRSQILMWSANKVFEELTDVERQFHKALYTVRTYLNCERYSIGLLDMTKEKEFYDEWPVKPGEVEPYKGPKTPDGREVIFYKIIDYILHGKEEIKVIPTPPMDHWTLISGLPTYVAENGFICNMLNAPADEYFTFQKGPVDETGWVIKNVLSLPIVNKKEDIVGVATFYNRKDGKPFDEYDEHIAETLTQFLGWSLLNTDTYEKMNKLENRKDIAQEMLMNHTKATPDE

SEQ ID NO: 36 - protein, 404 aa, bovine
LFELVQDMQENVNMERVVFKILRRLCSILHADRCSLFMYRQRNGVAELATRLFSVQPDSVLEDCLVPPDSEIVFPLDIGVVGHVAQTKKMVNVQDVMECPHFSSFADELTDYVTRNILATPIMNGKDVVAVIMAVNKLDGPCFTSEDEDVFLKYLNFGTLNLKIYHYSYLHNCETRRGQVLLWSANKVFEELTDIERQFHKAFYTVRAYLNCDRYSVGLLDMTKEKEFFDVWPVLMGEAQAYSGPRTPDGREILFYKVIDYILHGKEDIKVIPSPPADHWALASGLPTYVAESGFICNIMNAPADEMFNFQEGPLDDSGWIVKNVLSMPIVNKKEEIVGVATFYNRKDGKPFDEQDEVLMESLTQFLGWSVLNTDTYDKMNKLENRKDIAQDMVLYHVRCDREE

SEQ ID NO: 37 - protein, 401 aa, bovine
LLRDFQDNLQAEKCVFNVMKKLCFLLQADRMSLFMYRARNGIAELATRLFNVHKDAVLEECLVAPDSEIVFPLDMGVVGHVALSKKIVNVPNTEEDEHFCDFVDTLTEYQTKNILASPIMNGKDVVAIIMAVNKVDGPHFTENDEEILLKYLNFANLIMKVFHLSYLHNCETRRGQILLWSGSKVFEELTDIERQFHKALYTVRAFLNCDRYSVGLLDMTKQKEFFDVWPVLMGEAPPYAGPRTPDGREINFYKVIDYILHGKEDIKVIPNPPPDHWALVSGLPTYVAQNGLICNIMNAPSEDFFAFQKEPLDESGWMIKNVLSMPIVNKKEEIVGVATFYNRKDGKPFDEMDETLMESLAQFLGWSVLNPDTYELMNKLENRKDIFQDMVKYHVKCDNEE

SEQ ID NO: 38 - protein, 84 aa, bovine
EPLNIKDAYEDPRFNAEVDQITGYKTQSILCMPIKMHREEVVGVAQAINKKSGNGGTFTEKDEKDFAAYLAFCGIVLHMAQLYE

SEQ ID NO: 39 - protein, 81 aa, bovine
KIVNVPNTEEDEHFCDFVDTLTEYQTKNILASPIMNGKDVVAIIMAVNKVDGPHFTENDEEILLKYLNFANLIMKVFHLSY

SEQ ID NO: 40 - protein, 81 aa, bovine
KMVNVQDVMECPHFSSFADELTDYVTRNILATPIMNGKDVVAVIMAVNKLDGPCFTSEDEDVFLKYLNFGTLNLKIYHLSY

SEQ ID NO: 41 - protein, 81 aa, bovine
KTFNVPDVKKNSHFSDFMDKQTGYVTRNILATPIVMGKEVLAVFMAVNKVDASEFSKQDEEVFSKYLSFVSIILKLHHTNY

SEQ ID NO: 42 - protein, 81 aa, bovine
KSIQLKDLTSEDMQQLQSMLGCEVQAMLCVPVISRATDQVVALACAFNKLGGDLFTDQDEHVIQHCFHYTSTVLTSTLAFQ

SEQ ID NO: 43 - protein, 91 aa, bovine
EPLNIPDVSKDKRFPWTNENMGNINQQCIRSLLCTPIKNGKKNKVIGVCQLVNKMEETTGKVKAFNRNDEQFLEAFVIFCGLGIQNTQMYE

SEQ ID NO: 44 - protein, 84 aa, bovine
LICNIMNAPSEDFFAFQKEPLDESGWMIKNVLSMPIVNKKEEIVGVATFYNRKDGKPFDEMDETLMESLAQFLGWSVLNPDTYE

SEQ ID NO: 45 - protein, 84 aa, bovine
FICNIMNAPADEMFNFQEGPLDDSGWIVKNVLSMPIVNKKEEIVGVATFYNRKDGKPFVEQDEVLMESLTQFLGWSVLNTDTYD

SEQ ID NO: 46 - protein, 84 aa, bovine
FICNMLNAPADEYFTFQKGPVDETGWVIKNVLSLPIVNKKEDIVGVATFYNRKDGKPFDEYDEHIAETLTQFLGWSLLNTDTYE

SEQ ID NO: 47 - protein, 82 aa, bovine
QILNIPDAYAHPLFYRGVDDSTGFRTRNILCFPIKNENQEVIGVAELVNKINGPWFSKFDEDLATAFSIYCGISIAHSLLYK